(19) United States
(12) Patent Application Publication
(10) Pub. No.: US 2024/0198013 A1
(43) Pub. Date: Jun. 20, 2024

(54) SAFETY SHIELD FOR AN INJECTION DEVICE

(71) Applicant: West Pharmaceutical Services, Inc., Exton, PA (US)

(72) Inventors: Lawton LAURENCE, Chester Springs, PA (US); Lauren NUTT, Exton, PA (US); Jeff THAWLEY, Wilmington, DE (US)

(21) Appl. No.: 18/286,979

(22) PCT Filed: Apr. 14, 2022
(86) PCT No.: PCT/US2022/024866

§ 371 Date: Oct. 13, 2023

Related U.S. Application Data
(60) Provisional application No. 63/174,722, filed on Apr. 14, 2021.

(51) Int. Cl. A61M 5/32; A61M 5/20

(52) U.S. Cl. CPC A61M 5/3245; A61M 5/2033; A61M 5/3275; A61M 5/3293; A61M 2207/10

(57) ABSTRACT

An injection device includes a housing defining a longitudinal axis and receiving a medicament container containing a medicament. The housing has a distal end which defines an opening for receiving a portion of a needle operably coupled with the medicament container. A safety shield surrounds at least a distal portion of the housing. The safety shield advances distally relative to the housing from a retracted position to a deployed position for shielding the needle. An advancement spring is arranged between the housing and the safety shield. The advancement spring advances the safety shield from the retracted position to the deployed position, and interlocks with the housing and the safety shield when the safety shield is in the deployed position to prevent return of the safety shield to the retracted position.

### CROSS-REFERENCE TO RELATED APPLICATIONS

This application claims the benefit of U.S. Provisional Patent App. No. 63/174,722, filed Apr. 14, 2021, the disclosure of which is hereby incorporated by reference herein.

### FIELD

The present disclosure relates to an injection device arranged to shield a hypodermic needle to thereby prevent injury, and to an associated method.

### BACKGROUND

Injection devices, such as syringes and autoinjectors, use hypodermic needles to deliver medicaments subcutaneously. Because used hypodermic needles can harbour harmful pathogens, injection devices must be handled carefully and disposed of safely. If improperly used or disposed of, injection devices may cause injury, and may transmit disease.

It is known to provide injection devices with a safety shield configured to deploy following performance of an injection, thereby shielding the hypodermic needle to prevent harm. However, such safety shields and their deployment mechanisms can be mechanically complex. As a result, manufacture and assembly of such injection devices can be expensive. Furthermore, complex deployment mechanisms are vulnerable to failure, thereby reducing their efficacy at preventing harm.

Another challenge posed by current injection devices, and in particular autoinjectors, is that it can be difficult for a user or healthcare professional to determine whether or not a correct or complete dose of medicament has been delivered to a patient. This can be dangerous, because it could result in a patient receiving a dose of medicament that is either too small (if an injection is only partially completed and the user does not realise), or too large (if an injection is only partially completed, and is then then attempted for a second time with a fresh dose).

These challenges are particularly prevalent in autoinjectors for home use, because such autoinjectors are often used by patients (rather than medical professionals), and so there is a greater risk of misuse.

One type of known autoinjector includes a safety mechanism configured, once a predetermined dose of medicament has been delivered, to retract the syringe and thus the hypodermic needle within a housing of the injection device, such that the tip of the hypodermic needle is shielded within the housing after use. However, the retraction of the hypodermic needle can fail, such that the autoinjector is not rendered safe after use. Moreover, retraction of the syringe assembly after completion of an injection procedure shields the hypodermic needle only after completion of the injection.

As discussed above, type of known autoinjector includes a safety mechanism configured to advance a safety shield after use of the autoinjector.

Both types of autoinjectors discussed above ensure that the hypodermic needle is not exposed after an injection is completed. However, in each of these systems, the hypodermic needle may be exposed if the injection device is removed from the injection site prematurely (e.g. before the injection process has been completed) and further no indication is provided as to whether or not a required dose of medicament was delivered to the patient. Additionally, after use, the above-described types of autoinjectors may indicate a successful delivery even when the autoinjector was prematurely removed from the patient, which results in less than the entire dose actually being delivered to the intended subcutaneous location.

A need therefore exists for an improved injection device.

### SUMMARY

In an embodiment there is provided an injection device comprising:
a housing defining a longitudinal axis and configured to receive a medicament container containing a medicament, the housing having a distal end which defines an opening for receiving a portion of a needle operably coupled with the medicament container;
a safety shield surrounding at least a distal portion of the housing, the safety shield configured to advance distally relative to the housing from a retracted position, to a deployed position for shielding the needle; and
an advancement spring arranged between the housing and the safety shield, the advancement spring configured to advance the safety shield from the retracted position to the deployed position;

wherein the advancement spring is configured to interlock with the housing and the safety shield when the safety shield is in the deployed position, to thereby prevent return of the safety shield to the retracted position.

Because the advancement spring acts as a lockout means for the safety shield, as well as a deployment means for the safety shield, the injection device is mechanically simple. Accordingly, it is inexpensive to manufacture, and simple to assemble. Furthermore, because the deployment and lockout means comprise few components, the chance of device failure is low. The advancement spring may be a helical spring.

Herein, the distal end of a component denotes an end of the component that is arranged to be closest to an injection site during use; while the proximal end of a component denotes an end of the component that is arranged to be farthest from the injection side during use. Similarly, movement in a distal direction denotes movement towards the injection site during use; while movement in a proximal direction denotes movement away from the injection site during use.

The injection device may further comprise a handle portion attached to the housing and including drive components for performing an injection. Alternatively, the housing may be configured for attachment to a handle portion including drive components for performing an injection. For example, the injection device according to the above embodiment may be provided as a disposable component for attachment to a reusable handle portion.

The injection device may be provided without the medicament container, or with the medicament container disposed therein.

The safety shield may comprise a sleeve surrounding at least a distal portion of the housing. The sleeve may comprise a generally closed tubular body. The tubular body may be open at its proximal end (e.g. without a wall at its distal end). Alternatively, the safety sleeve can comprise a wall with an opening provided therein at its distal end, to allow the needle to extend through for injection.

The housing may comprise a first locking surface, and the safety shield may comprise a second locking surface arranged to face the first locking surface when the safety shield is in the deployed position. The advancement spring may be configured to interlock with the first and second locking surfaces when the safety shield is in the deployed position, to thereby prevent return of the safety shield to the retracted position. For example, at least a portion of the advancement spring may be configured to wedge between the first and second locking surfaces when the safety shield is in the deployed position.

The first locking surface may comprise a distal-facing shoulder of the housing, and the second locking surface may comprise a proximal-facing surface. The second locking surface may be located on an inner surface of the safety shield. A longitudinal ridge may extend in the distal direction from the proximal-facing surface. The safety shield (for example the longitudinal ridge of the safety shield) may be configured to radially compress the advancement spring when the safety shield is in the retracted position. The advancement spring may be configured to radially expand as it passes the second locking surface during advancement of the safety shield. This radial expansion may cause the advancement spring to interlock with the first and second locking surfaces as described above.

The advancement spring may comprise a proximal portion having a first diameter, and a distal portion having a second diameter that is smaller than the first diameter, wherein the proximal portion is configured to interlock with the housing and the safety shield when the safety shield is in the deployed position. The proximal portion of the advancement spring may be compressed into a third diameter that is smaller than the first diameter when the safety shield is in the retracted position, and wherein the advancement spring is arranged to expand to the first diameter when the safety shield is in the deployed position. The safety shield (for example the longitudinal ridge of the safety shield) may be configured to compress the proximal portion of the advancement spring into the third diameter when the safety shield is in the retracted position.

One of the housing and the safety shield may comprise a retaining tab. The other of the housing and the safety shield may comprise a one-way catch. The retaining tab may be configured to slide over the one-way catch during assembly of the injection device, and may further be configured to abut the one-way catch during advancement of the safety shield to thereby prevent separation of the safety shield from the housing. Accordingly, the retaining tab and the one-way catch facilitate easy assembly of the injection device (e.g. by sliding the safety shield in the proximal direction over the housing), while preventing separation of the safety sleeve from the housing when the safety shield advances distally.

The one-way catch may comprise a sloped surface arranged to facilitate the movement of the retaining tab over the one-way catch during assembly, and a sheer surface arranged to prevent the movement of the retaining tab over the one-way catch during advancement of the safety shield. The sloped surface may face in the distal direction. The sheer surface may face in the proximal direction. The one-way catch may further comprise a cantilever arm. The sloped and sheer surfaces may be located at a free end of the cantilever arm. In some examples, the housing comprises one-way catch and the safety shield comprises the retaining tab. In such examples, the retaining tab may comprise an circumferential protrusion. The injection device may comprise a plurality of cantilever arms, for example two cantilever arms, three cantilever arms, or four cantilever arms. Where there are a plurality of cantilever arms, they may be equally spaced from one another in a radial direction.

The housing and the shield may comprise a longitudinal groove. The longitudinal groove may be configured to receive the longitudinal ridge. This may prevent rotation of the safety shield relative to the housing.

The injection device may comprise a releasable locking mechanism configured, when engaged, to lock the safety shield in the retracted position.

The injection device may further comprise a needle hub coupled to the needle and the medicament container (e.g. a needle hub that is connected to the needle and operatively coupled to the medicament container). The needle hub may be slidably received within the housing. The needle hub may be moveable relative to the housing between a first (retracted) position in which it is recessed from the opening, and a second (injection) position in which it extends through the opening.

The needle hub may be configured to disengage the releasable locking mechanism when in the second (injection) position, thereby unlocking the safety shield.

For example, the releasable locking mechanism may comprise a latching surface connected to one of the safety shield and the housing, and a flexible latch arm connected to the other of the safety shield and the housing. The flexible latch arm may be configured to engage the latching surface to thereby lock the safety shield in the retracted position. The needle hub may be configured to disengage the flexible latch arm from the latching surface when in the second (injection) position, thereby unlocking the safety shield.

The injection device may comprise a return spring arranged to bias the medicament container and the needle hub into the first (retracted) position. The return spring may be positioned between the housing and the medicament container. The return spring may be a helical spring. The needle hub may be moveable into the second (injection) position during performance of an injection, e.g. under the influence of a drive component or plunger. The injection device may further be configured such that the needle hub returns to the first (retracted) position following completion of an injection.

The needle hub may be configured, in response to completion of delivery of a dose of medicament, to return to the first (retracted) position under the influence of the return spring. This in turn may lock the safety shield in the retracted position. However, if the injection device is removed from an injection site prior to completion of delivery of a dose of medicament, the safety shield may move to the deployed position before the needle hub returns to the first (retracted) position, even if the delivery of the dose of medicament is completed after removal of the injection device from the injection site. Because the injection device may react in a first manner (return of the medicament container to the first (retracted) position in response to completion of delivery of a dose of medicament), and in a second manner (advancing the safety shield) in response to premature removal of the injection device from the injection site, the activation state of the safety mechanism can provide an indication of the status of delivery of a dose of medicament. For example, the injection device may have the following indication states:
a first indication state, which indicates that the device was removed from the injection site after a complete dose of medicament was delivered, e.g. an indication that the injection device was not prematurely removed from the injection site;
a second indication state, which indicates that the device was removed from the injection site prematurely and an incomplete dose has been delivered; and
a third indication state, which indicates that the device was removed from the injection site before a complete dose of medicament was delivered, e.g. an indication that the injection device was removed prematurely from the injection site.

The first indication state may be a first post-injection configuration of the device. The third indication state may be a second post-injection configuration of the device. The second indication state may be a precursor to the second post-injection configuration. The three indication states may be visually distinct from one another. For example, the injection device may be configured such that movement of the needle hub from the second (injection) position to the first (retracted) position exposes a first visual indicator. The injection device may be further configured such that advancement of the safety shield exposes a second visual indicator.

In another embodiment there is provided an injection device comprising:
a housing defining a longitudinal axis and configured to receive a medicament container medicament container containing a medicament and a needle assembly for coupling with the medicament container, the housing having a proximal end which defines an opening for receiving a needle coupled with the medicament container;
a safety shield surrounding at least a proximal portion of the housing, the safety shield being proximally advanceable relative to the housing from a retracted position, to a deployed position for shielding the needle; and
an advancement spring positioned between the housing and the safety shield, the advancement spring configured to advance the safety shield;
wherein one of the housing and the safety shield comprises a retaining tab, and the other of the housing and the safety shield comprises a one-way catch;

the one-way catch configured to slide over the retaining tab during assembly of the injection device, and configured to abut the retaining tab during advancement of the safety shield to thereby prevent separation of the safety shield from the housing.

As the reader will understand, features described in respect of the above two embodiments are combinable.

In another embodiment there is provided an injection device comprising:
a housing configured to receive a medicament container and having a distal end which defines an opening for receiving a needle coupled with the medicament container;
a safety shield surrounding at least a distal portion of the housing, the safety shield being distally advanceable relative to the housing from a retracted position, to a deployed position for shielding the needle; and
an advancement spring positioned between the housing and the safety shield, the advancement spring configured to advance the safety shield from the retracted position to the deployed position;
wherein the safety shield comprises a latching surface, and the housing comprises a flexible latch arm coupled; the flexible latch arm configured to engage a latching surface of the safety shield to thereby lock the safety shield in the retracted position; and
wherein the flexible latch arm is configured to be disengageable from the latching surface during performance of an injection.

As the reader will understand, features described in respect of the immediately above-mentioned embodiment are combinable with at least the features of the first-mentioned embodiment above.

The housing may be arranged to receive a medicament container and an associated needle hub therein, the needle hub being attached to a needle and further being coupled with the medicament container. The housing may be further arranged such that the medicament container and needle hub are moveable relative to the housing between a first (retracted) position in which the needle hub is recessed from the opening, and a second (injection) position in which the needle hub extends through the opening. The needle hub may be configured to disengage the flexible latch arm from the latching surface when in the second (injection) position.

In another embodiment there is provided a method of manufacturing an injection device, the method comprising:
providing a housing defining a longitudinal axis;
providing a safety shield surrounding at least a distal portion of the housing, the safety shield being distally advanceable relative to the housing from a retracted position, to a deployed position for shielding the needle; and
arranging an advancement spring between the housing and the safety shield, the advancement spring configured to advance the safety shield;

wherein the advancement spring is configured to interlock with the housing and the safety shield when the safety shield is in the deployed position, to thereby prevent return of the safety shield to the retracted position.

For example, the above embodiment may be a method of manufacturing an injection device according to any of the preceding embodiments. The method may further comprise positioning the advancement spring within the safety shield before sliding the safety shield onto the housing.

### BRIEF DESCRIPTION OF THE DRAWINGS

The invention will now be described in more detail with reference to a number of non-limiting, exemplary embodiments shown in the following drawings, in which:

FIG. 1 shows a cross-sectional view of an injection device according to the present disclosure;

FIG. 2 shows a side-view of the injection device of FIG. 1, in a pre-injection configuration;

FIG. 3A shows an injection device according to the present disclosure in the storage state;

FIG. 3B shows the injection device of 3A in the storage state from an angle rotated 45 degrees about longitudinal axis L;

FIG. 4 shows the injection device of 3A in the ready state;

FIG. 5 shows the injection device of 3A at the point of activation;

FIG. 6 shows the injection device of 3A during activation but before medicament has been delivered;

FIG. 7 shows the injection device of 3A once the medicament has been delivered;

FIG. 8 shows the injection device of 3A when the drive has been released and the needle retracted;

FIG. 9 shows an exploded view of a power pack according to the present disclosure;

FIG. 10A shows an enlarged perspective view of a latch in accordance with an embodiment of the present disclosure;

FIG. 10B shows an enlarged perspective view of an alternative latch in accordance with the present disclosure;

FIG. 10C shows an enlarged perspective view of a latch extension in accordance with the present disclosure;

FIG. 11A shows an enlarged view of the proximal end of the drive assembly in accordance with the present disclosure, with the proximal housing in an unactuated position;

FIG. 11B shows an enlarged view of the proximal end of the drive assembly shown in FIG. 11A, with the proximal housing in an actuated position;

FIG. 12 shows an injection device according to a further embodiment of the present disclosure;

FIG. 13 shows an injection device according to a further embodiment of the present disclosure;

FIG. 14 shows a method of assembling an injection device comprising a power pack as described herein;

FIG. 15 is a cross-sectional view of a part of an injection device of FIG. 1;

FIG. 16a shows an isometric view of the damper of FIG. 15;

FIG. 16b shows an exploded view of the damper of FIG. 16a;

FIG. 16c show a cross section in a plane lying along the longitudinal axis of the damper of FIG. 16a;

FIG. 17a shows an isometric view of the first drive component of FIG. 15;

FIG. 17b show a cross section in a plane lying along the longitudinal axis of the first drive component of FIG. 17a;

FIGS. 18a-18e show cross-sectional views of the first drive component and damper in a plane lying along the longitudinal axis L in various states of retraction or extension of the first drive component within the injection device;

FIG. 19 shows a cross-sectional view of a further embodiment of the present disclosure;

FIG. 20 shows an isometric cross-sectional view of a further embodiment of the present disclosure;

FIG. 21 shows a cross-sectional view of a first drive component according to a further embodiment of the present disclosure;

FIGS. 22a-22d show isometric cross-sectional views in various planes spaced apart along the length of the first drive component of FIG. 21;

FIG. 23 shows an isometric cross-sectional view of a further embodiment of the present disclosure;

FIG. 24a shows an isometric view of an embodiment of the damper of FIG. 23;

FIG. 24b shows an isometric view of a further embodiment of the damper of FIG. 23;

FIG. 25 shows an isometric cutaway view of a further embodiment of the present disclosure;

FIG. 26a shows an end elevation view of the first drive component of FIG. 25;

FIG. 26b shows a side elevation view of the first drive component of FIG. 25;

FIG. 26c shows a cross-sectional view of the first drive component of FIG. 25;

FIG. 27 shows a cross-sectional view of a damper according to an embodiment of the disclosure;

FIGS. 28a-28d show cross-sectional views of four further embodiments of the present disclosure;

FIG. 29 shows, in schematic form, a method of assembling a device according to the disclosure;

FIG. 30a shows a cross-sectional view of a portion of the injection device of FIG. 1 in a pre-injection (storage) state;

FIG. 30b shows the assembly of FIG. 30a in a post-injection state (or during an injection);

FIG. 31a shows a cross-sectional view of another assembly for an injection device according to the present disclosure when the assembly is in a storage state;

FIG. 31b shows the assembly of FIG. 31a in a post-injection state (or during an injection);

FIG. 32a shows a cross-sectional view of another assembly for an injection device according to the present disclosure when the assembly is in a storage state;

FIG. 32b shows the assembly of FIG. 32a in a post-injection state (or during an injection);

FIG. 32c shows another view of the assembly of FIGS. 32a and 32b;

FIG. 33 shows a cross-sectional view of a further assembly for an injection device according to the present disclosure when the assembly is in a storage state;

FIG. 34 shows a cross-sectional view of a further assembly for an injection device according to the present disclosure when the assembly is in a storage state;

FIG. 35 shows a cross-sectional view of a further assembly for an injection device according to the present disclosure when the assembly is in a storage state;

FIG. 36 shows a cross-sectional view of a further assembly for an injection device according to the present disclosure when the assembly is in a storage state;

FIG. 37 shows a cross-sectional view of a further assembly for an injection device according to the present disclosure when the assembly is in a storage state;

FIG. 38 shows a cross-sectional view of a further assembly for an injection device according to the present disclosure when the assembly is in a storage state;

FIG. 39 shows a cross-sectional view of a further assembly for an injection device according to the present disclosure when the assembly is in a storage state;

FIG. 40 shows a further assembly for an injection device according to the present disclosure;

FIG. 41 shows a cross-sectional view of a further assembly for an injection device according to the present disclosure when the assembly is in a storage state;

FIG. 42 shows a cross-sectional view of a further assembly for an injection device according to the present disclosure when the assembly is in a storage state;

FIG. 43 shows a flow diagram of a method according to the present disclosure;

FIG. 44a shows a first cross-sectional view of a distal end of the injection device from FIG. 1;

FIG. 44b shows a second, perpendicular cross-sectional view of the distal end of the injection device from FIG. 1;

FIG. 44c shows a side-view of the distal end of the injection device from FIG. 1;

FIG. 45a shows a first perspective view of a safety shield from the injection device of FIG. 1;

FIG. 45b shows a second perspective view of the safety shield from FIG. 45a;

FIG. 45c shows a perspective view of a housing from the injection device of FIG. 1;

FIG. 46 shows an advancement spring from the injection device of FIG. 1;

FIG. 47a shows a storage configuration of the injection device from FIG. 1;

FIG. 47b shows a pre-injection configuration of the injection device from FIG. 1;

FIG. 47c shows a first mid-injection configuration of the injection device from FIG. 1;

FIG. 47D shows a first post-injection configuration of the injection device from FIG. 1;

FIG. 47E shows a second mid-injection configuration of the injection device from FIG. 1;

FIG. 47F shows a second post-injection configuration of the injection device from FIG. 1;

FIG. 48A shows an external view of the injection device of FIG. 1 in the first post-injection configuration of FIG. 47D;

FIG. 48B shows an external view of the injection device of FIG. 1 in the second mid-injection configuration of FIG. 47E;

FIG. 48C shows an external view of the injection device of FIG. 1 in the second post-injection configuration of FIG. 47F; and

FIG. 49 shows a method of assembling the injection device of FIG. 1.

Like reference numerals are used for like components and like embodiments throughout the detailed description.

### DETAILED DESCRIPTION

The present disclosure is directed generally to injection devices, assemblies for injection devices, and to methods of assembly or manufacture for those devices and assemblies. In a first aspect, the disclosure provides a power pack. The power pack may form part of a drive assembly. In a second aspect, the disclosure provides a damping mechanism for an injection device for damping the drive force provided by the power pack. In a third aspect, the disclosure provides a connection assembly for an injection device for connecting a needle hub with a medicament cartridge. In a fourth aspect, the disclosure provides a passive safety shield for an injection device for shielding the user from the exposed tip of the needle.

Each of the aspects is described in turn below. The aspects may be implemented independently of each other or in combination, as will be become apparent from the following detailed description. For example, any of the power pack embodiments described below may be combined with any of the damping mechanism embodiments described herein. However, the power pack may be implemented without the damping mechanism.

Equally, the damping mechanism described below may be implemented with an alternative power pack assembly to the ones described herein. Although the power pack and the damper may be implemented independently, additional advantages may be provided where a power pack described herein is provided in an injection device in combination with the damping mechanisms described below. In particular, power packs according to the disclosure may allow for a larger drive spring than conventional injection devices. The drive force from the larger drive spring may optionally be damped using a damping mechanism described herein.

The power pack and/or the damping mechanism described herein may also provide additional advantages when combined in an injection device with the passive safety shield arrangement of the present disclosure. Alternatively, the safety field arrangement may be provided in isolation from the other aspects set out in this disclosure. The safety shield arrangement of the present disclosure may optionally be implemented in an injection device of the type configured to extend a needle from the housing for injection, deliver a dose of medicament through the needle, and then retract the needle after use.

Finally, it will also be appreciated that any of the connection assemblies described herein may be implemented independently of the injection devices described in this disclosure. The connection assemblies described herein may be implemented in any device or sub-assembly in which a medicament container comprises a septum configured to be pierced by a needle. Although the connection assemblies described herein may be implemented independently of the other aspects described below, it will be understood that additional advantages may be provided where the connection assemblies described herein are combined with one or more of the other aspects of this disclosure. In particular, the connection assemblies described herein may provide additional advantages when combined with the passive safety shield arrangement described below.

In the description below, an injection device will be described that includes an embodiment of each of the aspects identified above: an example power pack, an example damping mechanism, an example connection assembly and an example safety shield mechanism.

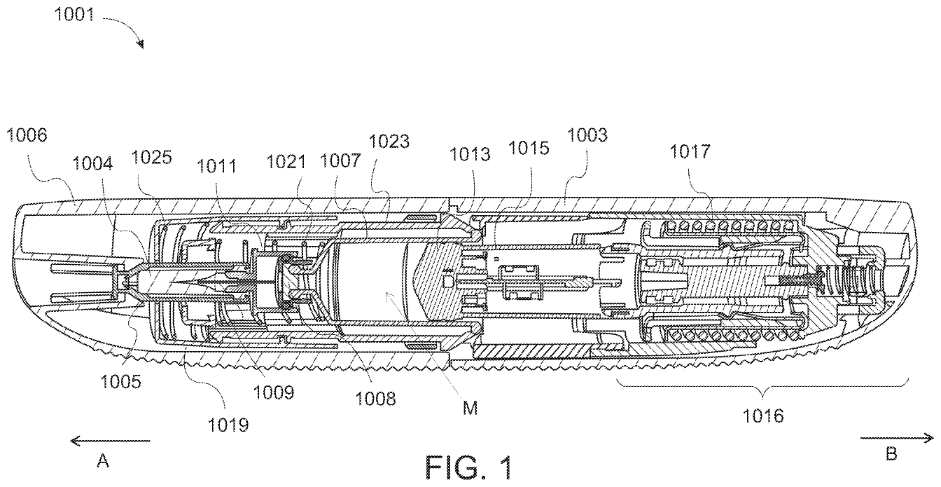

FIG. 1 shows a cross-sectional view of an injection device 1001 according to the present disclosure. The injection device 1001 includes a handle 1003 at a proximal end, and a cover 1006 at a distal end. The handle 1003 houses a drive assembly 1016 (which itself includes a drive spring 1017), and a plunger rod 1015. The distal direction is towards the needle-end of the device, as indicated by arrow A. The proximal direction is opposite the distal direction and is indicated by arrow B.

FIG. 1 shows the injection device 1001 in a storage configuration, in which the cover 1006 conceals the distal end of the injection device 1001. The cover 1006 is removeable from the injection device 1001, bringing with it needle cap 1005 and needle shield 1004, and thereby exposing the distal end of the injection device 1001. Included at the distal end of the injection device 1001 is a medicament container 1007, which is sealed at its proximal end by a plunger 1013, and sealed at its distal end by a septum 1008. A medicament M is contained within the medicament container 1007. Coupled to the distal end of the medicament container 1007 is a needle hub 1011, which itself is attached to a hypodermic needle 1009. The needle hub 1011 is translatable relative to the medicament container 1007. Accordingly, the septum 1008 is pierceable by the hypodermic needle 1009 in use so as to establish fluid communication between the medicament container 1007 and the hypodermic needle 1009 for performing an injection. Although embodiments of the disclosure herein are described with reference to an injection device comprises a medicament container in the form of a cartridge sealed by a septum, it will appreciated that in some embodiments the septum-sealed container may be replaced with a syringe comprising a needle.

With continued reference to FIG. 1, it is noted that the distal end of the hypodermic needle 1009 is recessed from the distal-most end of the housing 1023 in the configuration shown. A safety shield 1019 is also provided around the distal end of the housing 1023. The safety shield 1019 is in some situations advanceable relative to the housing 1023 after performance of an injection.

With continued reference to FIG. 1, the housing 1023 may be removeable from the handle 1003. Accordingly, the housing 1023 and its contents (i.e. the distal end of the injection device 1001) may be disposable.

To perform an injection, a user first removes the cover 1006 (and with it the needle cap 1005 and needle shield 1004) from the injection device 1001. The user then positions the distal end of the safety shield 1019 against the desired injection site, and actuates the drive assembly 1016. Once the drive assembly 1016 has been actuated, the plunger rod 1015 advances distally under the influence of the drive spring 1017. This in turn advances the needle hub 1011 and medicament container 1007 so that the hypodermic needle 1009 pierces the injection site. Continued advancement of the plunger rod 1015 then further advances the medicament container 1007 so that the septum 1008 is pierced by the hypodermic needle 1009; and finally advances the plunger 1013 through the medicament container 1007 and towards the septum 1008, thereby expelling medicament from the medicament container 1007 through the hypodermic needle 1009. An injection is thereby performed. Once the plunger rod 1015 has reached the end of its travel, the drive spring 1017 decouples from the plunger rod 1015, such that the return spring 1021 can then act to retract the medicament container 1007, needle hub 1011 and hypodermic needle 1009 in the proximal direction. The hypodermic needle 1009 is thereby retracted back into the housing 1023, thereby rendering the injection device 1001 safe following completion of the injection. In some situations, the safety shield 1019 will advance relative to the housing 1023 under the influence of the advancement spring 1025. The safety shield 1019 thereby provides an additional layer of safety.

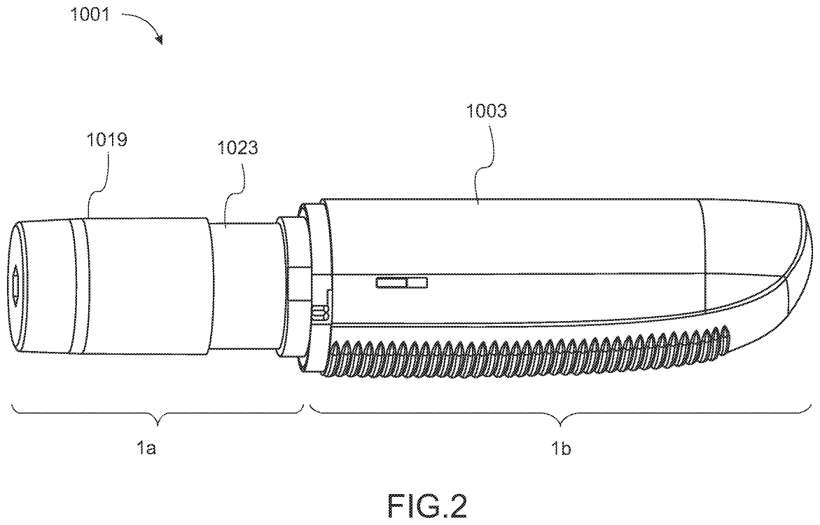

FIG. 2 shows an external view of the injection device 1001 of FIG. 1, in a pre-injection configuration in which the cover 1006 has been removed ready for use. Accordingly, the housing 1023 and safety shield 1019 are exposed ready for use. As shown, the injection device 1001 includes a proximal end 1a, and a distal end 1b. Distal end 1b may be removeable from proximal end 1a. In some embodiments, distal end 1b may be disposable and proximal end 1a may be reusable. In this way, the distal end of the device, which comprises the needle assembly, may be configured for a single use, whereas the proximal portion of the device which comprises the drive assembly may be reusable multiple times with a new distal portion being coupled to the proximal portion before each subsequent use of the device. Alternatively, both the proximal and distal portions of the device may be disposable, or both may be reusable.

### Power Pack

Power packs according to the first aspect of the disclosure will now be described.

The drive assembly 1016 shown in FIGS. 1 and 2 includes a power pack which is configured to drive the medicament container 1007 and the plunger rod 1015 distally under the influence of the drive spring 1017.

In general terms, the power pack includes the drive spring 1017 disposed within the housing 1023 and configured to provide a power source for driving the plunger rod 1015 and the medicament container 1007 distally to carry out an injection. The drive spring 1017 is coupled to the plunger rod 1015 via a releasable drive-lock mechanism (described in further detail below) that is configured to maintain the drive spring 1017 in engagement with the plunger rod 1015 during an injection process (so that the medicament container 1007 and/or the plunger rod 1015 move distally under the influence of the drive spring 1017), and to release the plunger rod 1015 from the influence of the drive spring 1017 after a dose of medicament has been delivered from the medicament container 1007.

Although the power pack will now be described in the context of an example injection device 1001 shown in FIG. 1, it will be appreciated that the power pack described herein may be employed in any injection device in which decoupling of the plunger rod 1015 from the drive spring 1017 is desired.

FIG. 1 shows the drive spring 1017 in the storage state. As shown in FIG. 1, prior to use, the drive spring 1017 is compressed into the storage state in which the drive spring 1017 stores elastic potential energy for driving the injection device 1001. The drive spring 1017 may be held in the storage state until a time when the injection device 1001 is activated and the drive spring 1017 is allowed to expand into an expanded state and, in the process, drive the plunger rod 1015 of the injection device 1001.

In general, the expansion of a drive spring 1017 in an injection device 1001 delivers a drive force that is inversely proportional to the degree of expansion of the spring 1017. To ensure that the drive spring 1017 delivers a drive force throughout the complete length of travel of a plunger rod 1015, the plunger rod 1015 ends its travel with the drive spring 1017 still somewhat compressed. Therefore, unless the medicament container 1007 and/or plunger rod 1015 are released from the influence of the drive spring 1017 during the injection process, the drive spring 1017 acts to pin the medicament container in a distal position within the housing. This may be undesirable in many cases, since it may make retraction of the medicament container 1007 within the housing 1023 after the injection has been completed more difficult.

As will be understood from reading the more detailed description below, power packs according to the present disclosure can allow the drive force of the drive spring 1017 to be reliably transferred to the plunger rod 1015 to deliver a dose of medicament during an injection process, and then subsequently disengaged from the plunger rod 1015 at the end of the travel of the drive spring 1017 so that the plunger rod 1015 (and the medicament container) are not pinned at the distal end of the injection device 1001 once the injection has finished.

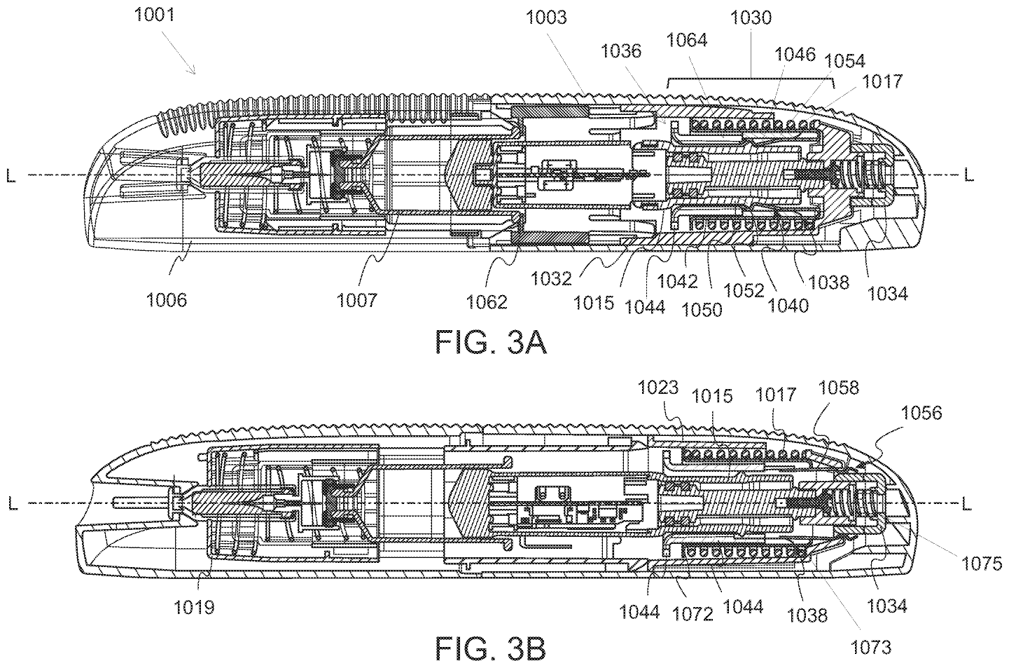

Embodiments of the power pack will now be described in more detail with reference to FIGS. 3A-13.

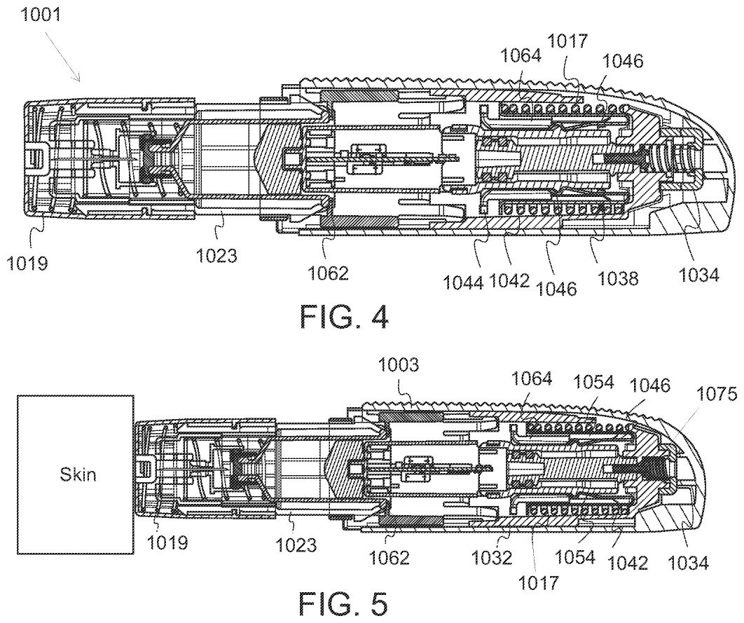

A first embodiment of an injection device 1001 comprising a power pack 1030 according to the disclosure is shown in FIGS. 3A-11B. FIG. 3A shows one cross-sectional view taken through the injection device 1001 along its longitudinal length. FIG. 3B shows another cross-sectional view taken through the injection device 1001, where the cross-section is taken at an angle 450 offset from FIG. 3A about the longitudinal axis L of the injection device 1001. FIGS. 3A and 3B show the device 1001 in the storage state prior to injection. FIGS. 4-8 show the injection device 1001 of 3A and 3B as the power pack 1030 advances the plunger rod 1015 and the medicament container 1007 distally within the housing, decouples the plunger rod 1015 from the influence of the drive spring 1017, and allows the medicament container 1007 to retract relative to the housing.

Turning first to FIG. 3A, the power pack 1030 comprises the drive spring 1017 and a drive-lock mechanism 1036 configured to engage the drive spring 1017 with the plunger rod 1015. The power pack 1030 is arranged within proximal housing 1032, which is movably mounted within the handle 1003 of the injection device 1001. The handle 1003 also houses an actuator 1034, which is explained in more detail with reference to FIG. 3B and FIGS. 12 and 13.

The drive spring 1017 here takes the form of a coiled helical spring. The helical spring is arranged concentrically with the plunger rod 1015 and the drive-lock mechanism 1036. The drive-lock mechanism 1036 comprises a latch mechanism 1038 (which comprises a latch 1040 and a latch extension 1042), and a retraction collar 1044.

As shown in FIG. 3A, the plunger rod 1015 may be a composite plunger rod comprising multiple different components. Alternatively, the plunger rod 1015 may be a monolithic body. The plunger rod 1015 may comprise a hollow proximal body portion, as shown in FIG. 3A. However, the plunger rod 1015 may also be substantially solid.

The latch mechanism 1038 is configured to engage the plunger rod 1015 with the drive spring 1017 and is also configured to maintain the drive spring 1017 in the storage state until the injection device 1001 is ready to use. The latch mechanism 1038 is at least partially received within an interior cavity of the drive spring 1017. Since the latch mechanism 1038 is located on the inside of the drive spring 1017, the diameter of the drive spring 1017 can be larger than if the drive spring 1017 were to be located inside the drive-lock mechanism 1036. Using a larger drive spring can deliver a higher drive force to the plunger rod 1015, allowing a high drive force to be delivered to the plunger rod 1015 throughout the injection process, to the end of the drive spring stroke.

As shown in FIG. 3A, the latch mechanism 1038 includes the latch 1040 fixed to the latch extension 1042, so that the latch 1040 and the latch extension 1042 can travel together under the influence of the drive spring 1017. The latch mechanism 1038 can be formed with a monolithic body (which includes both the latch and the latch extension) or it can be formed from a separate latch 1040 and a latch extension 1042 in engagement with each other.

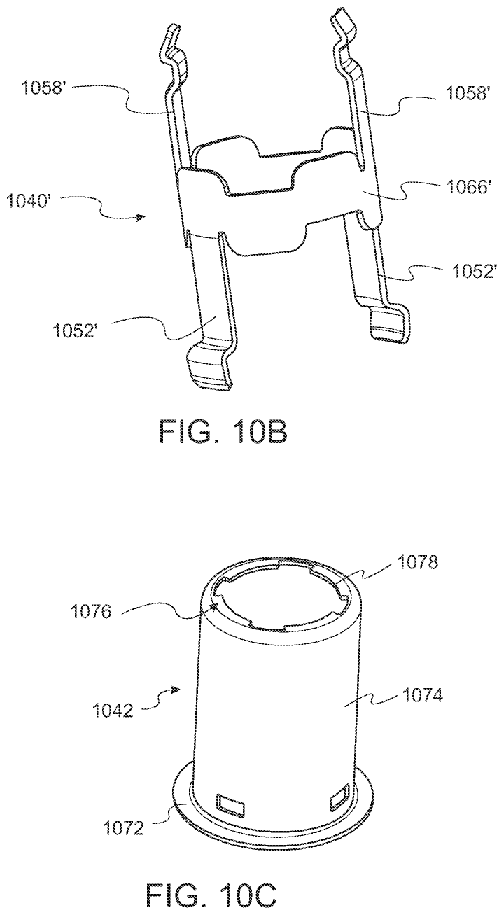

The latch 1040 comprises an engagement portion 1046 which is configured to engage the latch 1040 with the plunger rod 1015. The engagement portion 1046 here takes the form of an arm (or multiple arms) comprising a latching surface (shown in FIGS. 10A and 10B) configured to engage a corresponding latching surface 1050 on the plunger rod 1015. The arms of the engagement portion 1046 are shown clearly and indicated with reference numeral 1052 in FIG. 10A.

The arm(s) 1052 of the engagement portion 1046 are deflectable, in a radially outward direction, from the position in which they engage the corresponding latching surface 1050 on the plunger rod 1015 (as shown in FIG. 3A) to a position in which it no longer engages the corresponding latching surface 1050 on the plunger rod 1015. However, as shown in FIG. 3B, the engagement portion 1046 is prevented from deflecting outwardly by the retraction collar 1044 when the latch mechanism 1038 is in a drive-locked position, as described further below.

As shown in FIGS. 3A and 3B, the retraction collar 1044 is received between the engagement portion 1046 and the latch extension 1042. It comprises a generally tubular body, at least a portion of which forms a locking sleeve 1054. With the latch mechanism 1038 in the position shown in FIGS. 3A and 3B, the locking sleeve 1054 is configured to surround the arms 1052 of the engagement portion 1046 to prevent (or limit) radially outward flexing of the arms 1052, thereby holding the engagement portion 1046 in engagement with the plunger rod 1015. This position of the latch mechanism 1038 (in which the arms of the engagement portion 1046 are prevented from disengaging the plunger rod 1015) is the drive-locked position. Via this engagement, the latch mechanism 1038 releasably couples the drive spring 1017 to the plunger rod 1015, to allow drive from the drive spring 1017 to be transferred to the plunger rod 1015.

The retraction collar 1044 also comprises one or more recesses 1064. The recesses may be through-openings or closed recesses which provide a space into which the latch arms 1052 of the engagement portion 1046 can deflect to disengage the plunger rod 1015. The latch 1040 is slidably mounted with respect to the retraction collar 1044 such that relative movement between the locking sleeve 1054 and the arms 1052 of the engagement portion 1046 is possible once the retraction collar 1044 reaches a predetermined point. In the second position, the one or more recesses 1064 in the retraction collar 1044 are brought into register with the free ends of latch arms 1052 of the engagement portion 1046 such that the arms 1052 can flex outwardly. This position of the latch mechanism 1038 is the drive-unlocked position.

Operation of drive-lock mechanism 1036 during the course of an injection procedure is described in more detail later with reference to FIGS. 4-8.

Turning now to FIG. 3B, the latch mechanism 1038 may also engage with the actuator 1034 to retain the drive spring 1017 in the storage state until the injection device 1001 is activated. The latch 1040 of the latch mechanism 1038 may define at least one engagement element 1056 releasably secured within the handle 1003 to retain the drive spring 1017 in the storage state.

The engagement element 1056 can comprise a plurality of arms 1058 that extend in a proximal direction towards a free end. The arms 1058 can comprise latching surfaces configured to engage corresponding latching surfaces on the actuator 1034. The arms 1058 can be held in a position in which they engage the latching surface on the actuator 1034 by the proximal housing 1032.

With the distal end of the drive spring 1017 bearing on the distal flange 1072 of the latch extension 1042, fixation of the latch mechanism 1038 at the proximal end of the proximal housing 1032 prevents premature distal extension of the drive spring 1017.

As can also be seen in FIGS. 3A and 3B, the proximal end of the spring 1017 bears against an abutment surface 1073 on proximal housing 1032. In this way, in the storage state, the drive spring 1017 is compressed between a distal flange of latch extension 1042 and a proximal abutment surface of proximal housing 1032.

The proximal housing 1032 can be configured for relative movement with respect to the actuator 1034 between an unactuated position and an actuated position. This relative movement between the proximal housing 1032 and the actuator 1034 can be configured to release the arms 1058 of the engagement element 1056 from their confinement between the proximal housing 1032 and the actuator 1034, thereby allowing the drive spring 1017 to extend in a distal direction, driving the latch mechanism 1038 distally as it does so. The injector 1001 can include a spring 1075 disposed between the proximal housing 1032 and the actuator 1034, where the spring 1075 is configured to bias the proximal housing 1032 distally (into the unactuated position). During initiation of an injection, user force applied to the injector 1001 (as will be described below) must overcome the biasing force of spring 1075 in order to move the proximal housing 1032 proximally relative to the actuator 1034 so as to release the arms 1058 from engagement between the proximal housing 1032 and the actuator 1034.

The interaction between the engagement element 1056 and the actuator 1034 is described in more detail below with reference to FIGS. 10a-10c.

It will be appreciated that the latch mechanism 1038 can be configured in different ways. For example, the engagement portion 1046 of the latch 1040 can comprise one arm 1052 configured to couple the latch mechanism 1038 to the plunger rod 1015, or it may include a plurality of arms 1052, as shown. A plurality of arms 1052 may be distributed in diametrically opposed pairs or spaced circumferentially about the longitudinal axis L of the injection device 1001.

Similarly, the engagement element 1056 of the latch 1040 can comprise one arm 1058 configured to engage the actuator 1034, or it may include a plurality of arms 1058, as shown. A plurality of arms 1058 may be distributed in diametrically opposed pairs or spaced circumferentially about the longitudinal axis of the device. The latch mechanism 1038 may be formed of a resiliently deformable material, such as a metallic material or a resiliently deformable polymer.

The operation of the latch mechanism 1038 as an injection procedure progresses will now be described in more detail.

Starting with the device as shown in FIG. 4, the cover 1006 (shown in FIG. 1) is removed from the device 1001 shown in FIGS. 3a and 3b. The injection device 1001, in an unfired state in FIG. 4, is located on an injection site and the safety shield 1019 pressed against the injection site.

As shown in FIG. 5, the action of pressing the safety shield 1019 against an injection site causes the housing 1023 and the drive assembly 1016 (which includes the power pack 1030 and the proximal housing 1032) to move proximally within and relative to the handle 1003 (compressing spring 1075). It will be understood that the proximal movement of the housing 1023 related to the handle 1003 is equivalent to distal movement of the handle 1003 relative to the housing 1023. In other words, since the actuator 1034 is fixed relative to the handle 10013, rearward movement of the proximal housing 1032 relative to the handle 1003 moves the proximal housing 1032 in a distal direction relative to the actuator 1034 from the unactuated position to the actuated position, thereby releasing the engagement element 1056 from its confinement between the proximal housing 1032 and the actuator 1034.

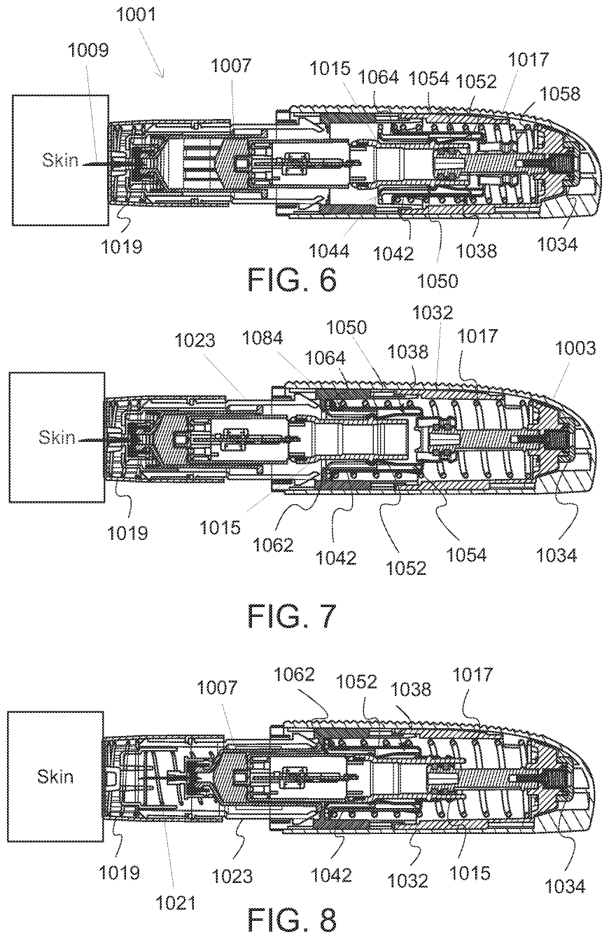

It should be noted that rearward movement of the proximal housing 1032 in the embodiment depicted is caused by the user pressing the injection device 1001 against the injection site. This action causes the housing 1023 to move in a proximal direction relative to the handle 1003. The rearward movement of the housing 1023 in turn is transferred to the proximal housing 1032 via intermediate housing 1084. However, the skilled person will appreciate that other arrangements are possible. For example, the proximal housing and the intermediate housing may be formed as a monolithic body. Alternatively, each of the proximal housing and/or the intermediate housing may be formed of multiple components. As shown in FIG. 6, once the injection device 1001 has been activated, the drive spring 1017 expands and drives the plunger rod 1015 distally. Distal movement of the plunger rod 1015 drives the medicament container 1007 distally to insert the needle 1009 into the injection site. Once the medicament container 1007 can travel no further in the distal direction, the plunger rod 1015 keeps advancing (moving distally now relative to the medicament container 1007) to discharge a dose of medicament from the medicament container 1007 through the needle 1009.

As can be seen from FIGS. 4-6, during this phase of the injection, the retraction collar 1044 moves with the latch mechanism 1038 so that the locking sleeve 1054 holds the arms 1052 of the engagement portion in a position in which they engage the latching surface(s) 1050 on the plunger rod 1015.

When the retraction collar 1044 has travelled as far in the distal direction as it can go, it stops upon coming into contact with an abutment 1062 provided in the housing 1023 (see FIG. 7). In the embodiment shown, the abutment is provided on intermediate housing 1084, which is configured to mate with the proximal housing 1032. However, the skilled person will appreciate that the abutment 1062 may be provided on another component such as the housing 1023 or the handle 1003.

Once the retraction collar 1044 has reached the abutment 1062, the latch mechanism 1038 continues its distal travel, advancing relative to the retraction collar 1044 (as shown in FIG. 7). The advancement of the latch mechanism 1038 relative to the retraction collar 1044 moves the arms 1052 of the engagement portion 1046 out of contact with the locking sleeve 1054 and into register with a recess 1064 or opening in the retraction collar 1044. In this position, with the locking sleeve 1054 no longer holding the arms 1052 of the engagement portion 1046 in engagement with the latching surface on the plunger rod 1015, the arms 1052 flex outwardly, thus disengaging the plunger rod 1015. As a result, the drive-lock mechanism 1036 (labelled in FIG. 3A and comprised of the retraction collar 1044 and the latch mechanism 1038) transitions from the drive-lock position to the drive-unlocked position, which decouples the plunger rod 1015 from the latch mechanism 1038 and decouples the plunger rod 1015 from the influence of the drive spring 1017.

As shown in FIG. 7, the latching surfaces of the plunger rod 1015 and the latch arms 1052 are angled such that the force of the latch 1040 pushing against the plunger rod 1015 (acting in the distal direction) acts to bias the latch arms 1052 outwardly and the latch arms are only prevented from flexing under this bias by the locking sleeve 1054 of the retraction collar 1044. Therefore, once the arms 1052 are brought into register with the recess 1064, the force of the drive spring 1017 causes them to disengage the plunger rod 1015, freeing the plunger rod 1015 from the influence of the drive spring 1017.

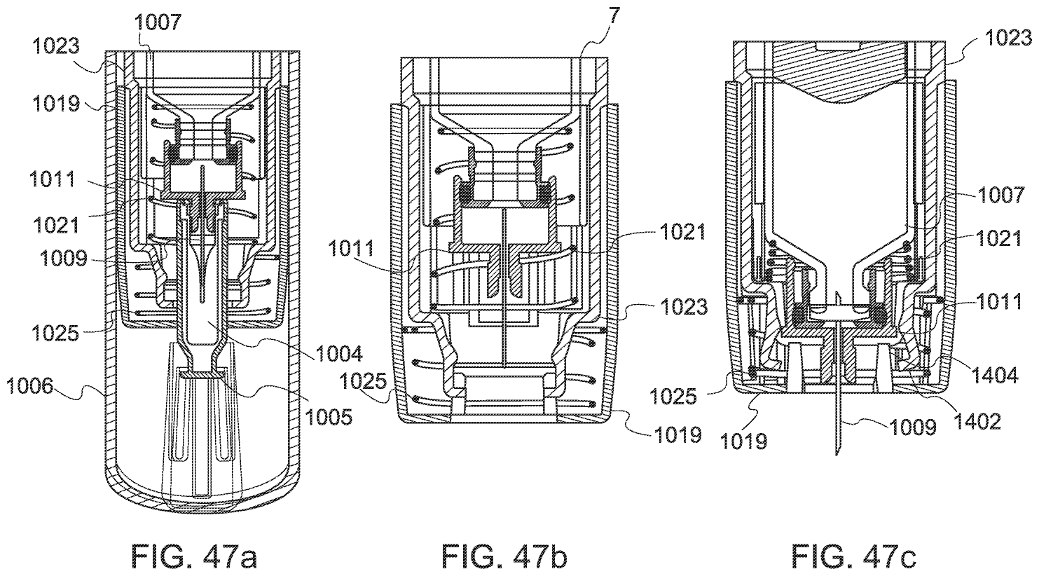

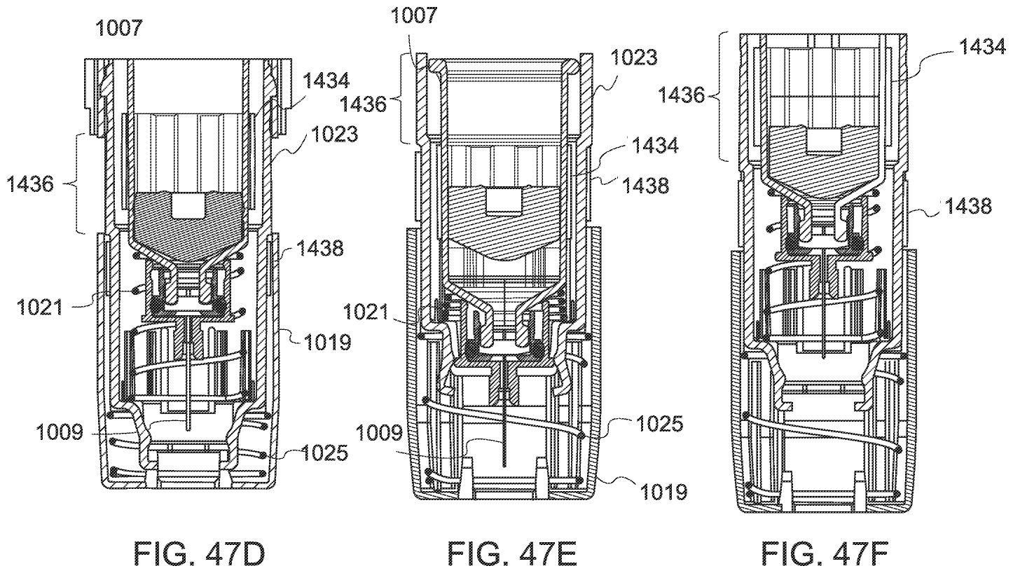

Finally, and as shown in FIG. 8, with the plunger rod 1015 free of the influence of the drive spring 1017, the medicament container 1007 can be retracted into the injection device 1001 after use, for example under the influence of a return spring 1021. A possible retraction mechanism for retracting the medicament container within the housing is described in more detail with reference to FIGS. 47a-47f.

Therefore, in the manner described above, the power pack of the present disclosure allows a drive spring to transmit drive force to a plunger rod via a drive-lock mechanism, which is configured to provide a releasable physical connection between the drive spring a and the plunger rod. In the first drive-locked configuration, expansion of the drive spring a causes the plunger rod to move under the force of the drive spring. In the second, drive-unlocked configuration, the drive-lock mechanism releases the force of the drive spring from the plunger rod. Once the force of the drive-spring is released from the plunger rod, the plunger rod is free to move without being pinned by the drive force of the drive spring.

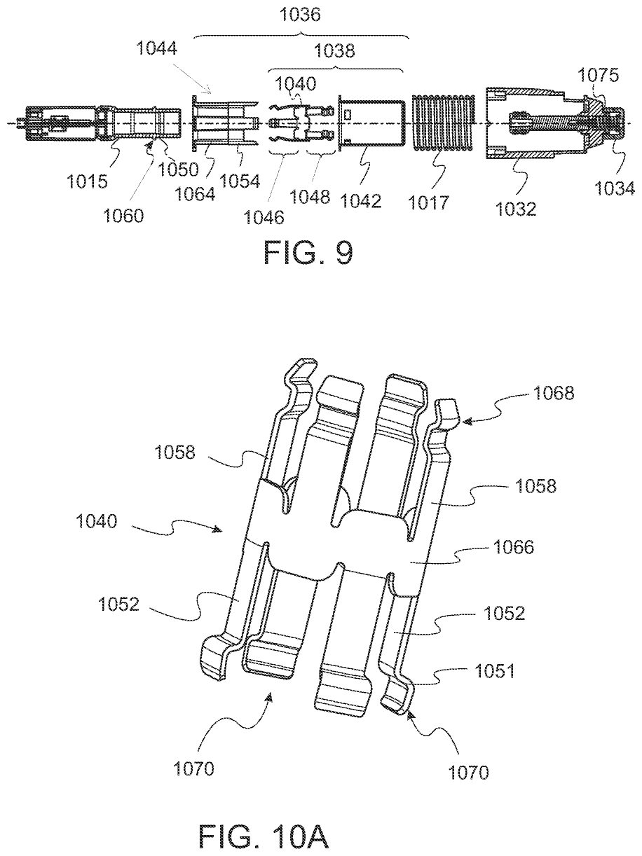

Turning to FIG. 9, the components of the power pack 1030 described with reference to FIGS. 3a-8 are shown in more detail in an exploded view. As shown in FIG. 9, the proximal housing 1032 can be assembled coaxially with the drive spring 1017, the latch mechanism 1038 comprising the latch 1040 and the latch extension 1042, the retraction collar 1044, and the plunger rod 1015. The drive spring 1017 is sized so that it fits within the inner diameter of the proximal housing 1032. The latch extension 1042 is sized to fit within the inner diameter of the coiled drive spring 1017. The latch 1040 is sized to fit within (and mate with) the latch extension 1042, with the retraction collar 1044 located between the latch 1040 and the latch extension 1042. Finally, the plunger rod 1015 is sized to fit within the inner diameter of the latch 1040.

The proximal housing 1032 is moveably mounted relative to the actuator 1034. The spring 1075 is configured to bias the proximal housing 1032 distally (into the unactuated position). In other words, the spring 1075 is positioned between the proximal housing 1032 and the actuator 1034 so that the spring 1075 biases the actuator 1034 and the proximal housing 1032 away from each other.

The retraction collar 1044 fits into the latch mechanism 1038 sliding between the latch 1040 and the latch extension 1042. The latch 1040 and the latch extension 1042 are pressed into radial engagement with the retraction collar 1044, such that the friction between the latch mechanism 1038 and the retraction collar 1044 resists longitudinal sliding of the latch mechanism 1038 relative the retraction collar 1044. The resultant interference fit between the retraction collar 1044 and the latch mechanism 1038 is predetermined to provide enough friction to resist longitudinal sliding of the latch mechanism 1038 and retraction collar 1044 relative one another during storage or in the initial stages of their distal movement in use, but low enough that the friction is overcome when the retraction collar 1044 meets the abutment (shown with reference numeral 1062 in FIG. 7).

In this exploded view, the arms that form the engagement portion 1046 of the latch 1040 can be clearly seen. The arms that form the engagement element 1056 of the latch 1040 can also be clearly seen. For clarity, the reference numerals that refer to the engagement portion arms 1052 and the engagement element arms 1058 are shown in FIG. 10A). The latching surfaces at the free ends of the arms are also visible in this view, as are the corresponding latching surface on the plunger rod 1015. However, for clarity the latching surfaces are labelled in FIG. 10A, which more clearly shows the latching surfaces of the arms. In the embodiment shown, the latching surface on the plunger rod 1015 is formed as an annular rib 1060 extending circumferentially around the plunger rod 1015. However, the skilled person will appreciate that this continuous circumferential rib 1060 can be replaced with a plurality of discrete latching surfaces.

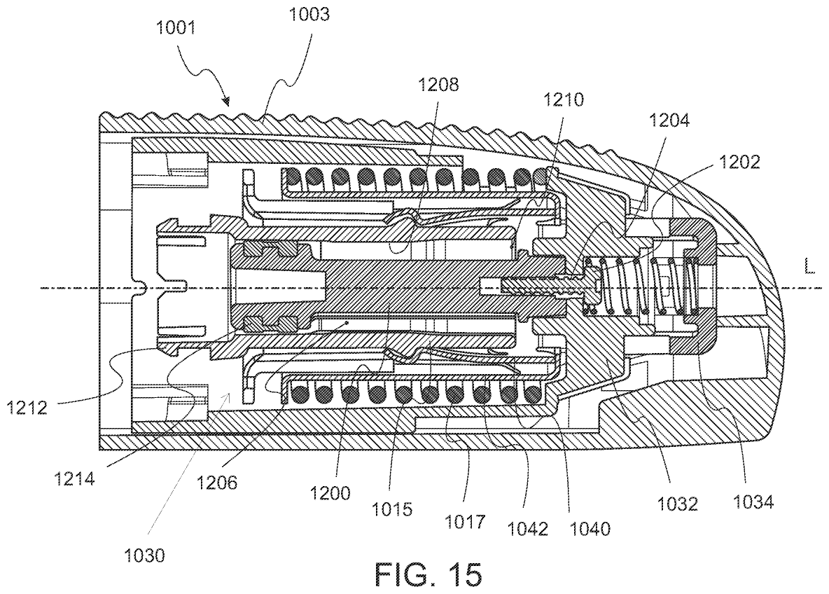

The force applied by the drive spring and which is released from acting on the plunger when the plunger has reached the end of travel may be more finely controlled by the use of a damping mechanism which is shown and described in more detail later with reference to FIGS. 15 to 29.

Turning now to FIGS. 10a and 10b, two example latch configurations will be described. FIG. 10A shows an example latch 1040 as shown in FIG. 9. This latch 1040 comprises four latch arms 1058 that make up the engagement element 1056 of the latch 1040, and four latch arms 1052 that make up the engagement portion 1046 of the latch 1040.

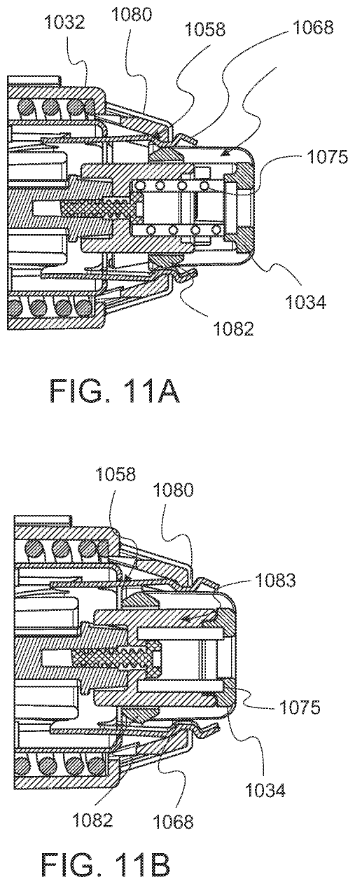

The latch arms 1058 that make up the engagement element 1056 extend in a proximal direction from a body 1066 of the latch 1040 toward respective free ends. At or towards the free end of each arm 1058, a locking hook 1068 is provided. The locking hook 1068 is the portion of the latch 1040 that is confined between the actuator 1034 and the proximal housing 1032. The confinement of the locking hook 1068 between the actuator 1034 and the proximal housing 1032 is explained in more detail in FIGS. 11a and 11b.

The latch arms 1052 that make up the engagement portion 1046 of the latch 1040 extend in a distal direction from the body 1066 of the latch towards respective free ends. At or towards the free end of each arm 1052, a shoulder 1070 is formed, on which the latching surface 1051 is located.

In the example shown in FIG. 10A, the engagement element 1056 includes four arms 1058 and the engagement portion 1046 includes four arms 1052. The four engagement portion arms 1052 and four engagement element arms 1056 are spaced around the body 1066, and in an alternating configuration such that engagement element arms 1058 are rotationally offset from engagement portion arms 1052 by 45°.

The latch arms 1052 and 1058 are configured to flex to allow them to disengage their respective latching surfaces during the course of the injection process. The latch arms 1052 and 1056 may therefore be formed of a resiliently deformable material.

FIG. 10B shows a variation of the latch 1040 shown in FIG. 10A. The latch 1040′ of FIG. 10B includes two engagement element arms 1058′ and two engagement portion arms 1052′. The engagement element arms 1058′ are diametrically opposed, and the engagement portion arms 1052′ are diametrically opposed. The engagement element arms 1058′ and the engagement portion arms 1052′ are rotationally offset in a staggered configuration. Any number of engagement element arms 1058′ and engagement portion arms 1052′ may be provided in combination, not limited to the specific examples shown in the figures. For example, six engagement element arms 1058′ and six engagement portion arms 1052′ may be provided, or two engagement element arms and four engagement portion arms may be provided.

FIG. 10C shows the latch extension 1042 in isolation. The latch extension 1042 comprises a distal flange 1072 extending radially outwardly and which provides an engagement surface for the drive spring 1017 to abut against and transfer force to the latch mechanism 1038. The latch extension 1042 comprises an extension sleeve 1074 which extends from the distal flange 1072 proximally to a castellated flange 1076. The castellated flange 1076 includes at least one castellation 1078 which extends radially inwardly from the extension sleeve 1074. When the latch 1040 is slotted into the latch extension 1042, the engagement element arms 1058 of the latch 1040 pass between the castellations 1078 such that the castellations 1078 bear against the body 1066 of the latch 1040 to transfer the force of the drive spring 1017 to the latch 1040 and subsequently through the engagement portion arms 1052 to the rib 1060 of the plunger rod 1015.

In the embodiment shown in FIG. 10C, the castellated flange 1076 comprises four castellations, with four spaces therebetween through which the four arms 1058 of the engagement element 1056 can extend. It will be appreciated that the number of castellations 1078 (and corresponding interstitial spaces) can be adapted to suit the number of engagement element arms 1058 provided on the latch 1040. The skilled person will also appreciate that the number of arms need not be equal to the number of spaces—more spaces than arms may be provided.

Turning now to FIGS. 11a and 11b, the operation of the actuator 1032 to release the latch 1040 will now be explained in more detail.

FIG. 11A shows a cross sectional view of the proximal end of the drive assembly, with the engagement element 1046 of the latch 1040 confined between the actuator 1032 and the proximal housing 1034 in an unactuated position, as it is when the device is in the storage state.

As can be seen in FIG. 11A, the arms 1058 of the engagement element 1056 extend through an opening in the proximal end of the proximal housing 1032. The locking hook 1068 of the arms 1058 are prevented from radially outward movement by a holding cap 1080, which fits over the proximal end of the proximal housing 1032. However, it will be appreciated that the body of the proximal housing 1032 itself may be sized to prevent outward flexing of the arms 1058.

With the actuator 1034 in the unactuated position shown in FIG. 11A, inward flexing of the arms 1058 is prevented by a stop surface 1082 of the actuator 1034. The shape of the locking hook 1068 prevents the arms 1058 from sliding in a longitudinal direction relative to the proximal housing. In this way, when the actuator 1034 is in the unactuated position shown in FIG. 11A, the arms 1058 are confined between the actuator 1034 and the proximal housing 1032, prevent forward movement of the latch mechanism 1038, and thereby holding the drive spring 1017 in its compressed state.

The proximal housing 1032 is maintained in the distal (unactuated) position relative to the actuator 1034 by the spring 1075 (shown schematically in FIGS. 11a and 11b). To actuate the device, a user presses the device 1001 against an injection site, which moves the housing 1023 in a proximal direction relative to the handle 1003. This in turn, moves the proximal housing 1032 proximally within the handle 1003, compressing the spring 1075.

FIG. 11B shows the proximal housing 1032 in the actuated position (in which the proximal housing 1032 has been moved in a proximal direction relative to the actuator 1034 by pressing the injection device 1001 against an injection site). In this position, the stop surface 1082 has been moved distally relative to the proximal housing 1032 (compressing the spring 1075) and the locking hook 1068 of the arm 1058 has been brought into register with an opening 1083 or recess in the actuator 1034. With the actuator 1034 in this position, the arms 1058 are free to flex inwardly (into the recess) so that the locking hook 1068 disengages the holding cap 1080 (or the proximal housing 1032), allowing the latch mechanism 1038 to move forward under the influence of the drive spring 1017.

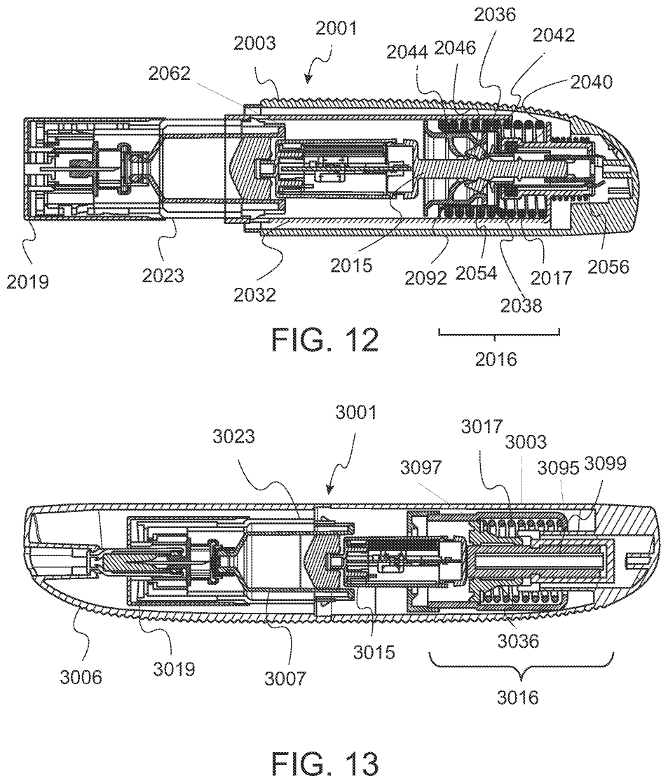

It will be understood that the releasable drive-lock mechanism described above and configured to release the plunger rod 1015 from the influence of the drive spring may also take different forms. In a further embodiment shown in FIG. 12, there is provided an injection device 2001 shown in an unfired state. The injection device 2001 comprising a handle 2003, a housing 2023, and a safety shield 2019, similar to the arrangement described above. The device 2001 also comprises a drive assembly 2016, which includes a power pack arranged within proximal housing 2032. The power pack includes a drive spring 2017 configured to be releasably coupled by a drive-lock mechanism 2036 to a plunger rod 2015.

The drive-lock mechanism 2036 of FIG. 12 comprises a retraction collar 2044 and a latch mechanism 2038, which is configured to releasably couple the plunger rod 2015 and the drive spring 2017.

Like the embodiment described above, the latch mechanism 2038 comprises a latch 2040 configured to engage a latching surface on the plunger rod 2015 and a latch extension 2042 having a flange against which the drive spring 2017 bears.

The construction of the latch mechanism of FIG. 12 differs from the arrangement described with reference to FIGS. 3a-11b, but the mode of operation is similar, as is described below.

The latch mechanism 2038 includes an engagement portion 2046 configured to releasably engage the plunger rod 2015 via deflectable latch arms configured to engage a corresponding latch surface on the plunger rod 2015. The latch mechanism 2038 also includes an engagement element 2056 configured to interact with an actuator to releasably hold the latch mechanism 2036 at the proximal end of the device, with the drive spring 2017 in the storage state.

The drive-lock mechanism 2036 also comprises a retraction collar 2044 which holds the latch arms of the engagement portion 2046 against the corresponding latching surface of the plunger rod 2015 by preventing outward deflection of the latch arms. The retraction collar 2044 shown in FIG. 12 differs from the retraction collar 2044 in that rather than comprising a cylindrical body having a recess into which the latch arms of the engagement portion can deflect, the retraction collar 2044 of FIG. 12 comprises a locking sleeve 2054 having a first inner diameter, which holds the latch arms of the engagement portion 2046 in engagement with the plunger rod 2015. Distal to the locking sleeve portion 2054, the retraction collar has a wider inner diameter (relative to the locking sleeve), which provides space for the latch arms of the engagement portion 2046 to deflect radially outwardly. The retraction collar 2044 also comprises a biasing surface 2092 distal to the locking sleeve portion 2054, which acts to move the latch arms of the engagement portion 2046 out of engagement with the plunger rod 2015 when the retraction collar 2044 is moved proximally relative to the latch mechanism 2038.

The operation of the device 2001 is similar to the operation of device 1001, as described below.

At the start of an injection procedure, the device 2001 is located on an injection site and the safety shield 2019 pressed against the skin. The action of pressing the safety shield 2019 against an injection site causes the housing 2023 and the drive assembly to move backwards within the handle 2003 and activate the device by compressing actuator (not shown) between the drive assembly 2016 and the handle 2003. The actuator in turn activates the injection device 2001 by unlocking the drive spring 2017 from containment in the storage state. Once the injection device 2001 has been activated, the drive spring 2017 expands and drives the plunger rod 2015 distally.

The injection device includes a drive-lock mechanism 2036 comprising a latch mechanism 2038 and a retraction collar 2044. The drive-lock mechanism 2036 in FIG. 12 operates in a similar way to the drive-lock mechanism 1036 described with reference to FIG. 3A.

During a first phase of the injection procedure (when the drive-lock mechanism is in a ‘drive-locked’ configuration), the relatively narrow inner diameter of the locking sleeve 2054 holds engagement portions 2046 of the latch 2040 in engagement with the plunger rod 2015. However, rather than comprising a generally cylindrical body with a recess for the latch arms of the engagement portion 2046 to flex into (like the embodiment described above with reference to Figures C3-C10), the retraction collar 2044 of the injection device 2001 in FIG. 12 includes biasing surface(s) 2092 which is configured to provide a radially outward bias to actively disengage the engagement portion 2046 from the plunger rod 2015, by deflecting the arms of the engagement portion 2046 into an inner section of the retraction collar 2044 that has a larger diameter than the locking sleeve portion 2054.

The biasing surface(s) 2092 may be configured as a plurality of arms, or as a conical ramp. When the retraction collar 2044 reaches the abutment 2062 in the housing, the retraction collar 2044 slides relative the latch mechanism 2038 (as the latch mechanism 2038 continues its forward travel) and pushes the engagement portion 2046 out of engagement with the plunger rod 2015. By first holding the engagement portion 2046 in engagement with the plunger rod 2015, and then providing an active decoupling of the engagement portion 2046 from the plunger rod 2015, the engagement portion 2046 may be configured in such a way that it either locks onto the plunger rod 2015 prior to disengagement or such that it engages the plunger rod 2015 with a strong grip. In this way, reliable engagement and reliable decoupling of the latch mechanism 2038 with the plunger rod 2015 can be achieved.

Accordingly, in one aspect of the disclosure, the retraction collar may therefore comprise a biasing surface configured to push the engagement arms radially outwardly to disengage the engagement arms from the plunger rod. In a further aspect, the biasing surface can comprise a plurality of arms or a conical ramp. It will be appreciated that these features may also be combined with a locking sleeve as described above.

FIG. 13 shows an alternative injection device 3001. The injection device 3001 includes a handle 3003 at a proximal end, a housing 3023, and a cover 3006 at a distal end. The handle 3003 houses a drive assembly 3016 which includes a drive spring 3017 and an advancement spring 3099. The drive spring 3017 provides the drive force for expelling the medicament from the medicament container 3007, but does not provide the driving force for moving the medicament container 3007 distally or piercing the skin. Drive force for moving the medicament container 3007 distally and piercing the skin is provided by a separate advancement spring 3099. When the injection device 3001 is activated, the drive spring 3017 remains in the compressed condition, whilst the advancement spring 3099 is released from the compressed condition.

The injection device 3001 may be activated by pressing the safety shield 3019 against the injection site or skin. The action of pressing the safety shield 3019 against an injection site causes the housing 3023 and the drive assembly 3016 to move backwards within the handle 3003 and activate the device 3001 by unlocking the advancement spring 3099 from the compressed state.

Once the injection device 3001 has been activated, the advancement spring 3099 expands and drives the plunger rod 3015 distally. Under the influence of the advancement spring 3099, the plunger rod 3015 travels in a distal direction, together with the drive spring 3017, which is maintained in its compressed state as shown in FIG. 13, between a distal abutment surface 3097 and a proximal abutment surface 3095, which are located at a fixed distance relative to each other. Once the advancement spring 3099 has travelled a predetermined distance, sufficient to fully advance the medicament container 3007 but not to deliver the medicament, the distal abutment surface 3095 is released from its fixed position relative to the proximal abutment surface 3097 and the drive spring is no longer confined in its compressed state. Once the distal abutment surface 3097 is decoupled from the proximal abutment surface 3095, the drive spring 3017 bears against the distal abutment surface 3097 and drives it, and in turn plunger rod 3015, in a distal direction. In this way, drive spring 3017 is allowed to expand after the advancement spring 3099 has advanced the medicament container 3007 to an injection position and provides additional driving force to the plunger rod 3015 through a drive-lock mechanism 3036. At or towards the end of travel of the drive spring 3017, the drive spring 3017 is decoupled from the plunger rod 3015 by a releasable drive-lock mechanism 3036. The drive-lock mechanism 3036 may be a releasable drive-lock mechanism similar to the releasable drive-lock mechanisms 1036, 2036 described above or an alternative drive assembly arrangement may be used.

Once the plunger rod 3015 has moved distally to the end of its travel (i.e. the medicament has been fully delivered) the drive-lock mechanism 3036 can release the plunger rod 3015 from the drive force of the drive spring 3017.

The predetermined distance for the advancement spring 3099 to expand before the drive spring 3017 is allowed to expand may be determined as the amount of travel required for the medicament container 3007 and needle to move from a storage position to a fully deployed position. In some cases the travel may be between 10 mm and 20 mm in the distal direction, for example, 18 mm.

By providing a separate advancement spring 3099, the drive spring 3017 may not be limited to a reduced power by the requirements of the needle advancement. For example, using a powerful drive spring to both move the needle distally to penetrate the injection site and to deliver the medicament may, in some circumstances cause, user discomfort. An advancement spring 3099 which delivers a lower force than the drive spring 3017 may result in a device which may gently advance the needle, but provide quick delivery of the medicament.

Accordingly, in one aspect, there is provided an injection device comprising: a housing defining a longitudinal axis; a drive spring disposed within the housing, the drive spring having a distal end and a proximal end opposite the distal end along the longitudinal axis, and the drive spring defining an interior cavity; a plunger disposed at least partially within a medicament container; a plunger rod attached to the plunger; an advancement spring having a distal end and a proximal end opposite the distal end along the longitudinal axis; an activation mechanism configured to retain the drive spring in a compressed condition, and to retain the advancement spring in a compressed condition; wherein the activation mechanism is configured upon activation of the injection device to release the advancement spring from the compressed condition to advance the medicament container to a distal position prior to releasing the drive spring from the compressed condition to drive a plunger in the medicament container and expel a medicament from the syringe.

In a further aspect the injection device comprises a drive-lock mechanism including: a latch mechanism at least partially received within the interior cavity and comprising at least one engagement portion configured to releasably engage the plunger rod, wherein the latch mechanism is configured to move distally under action of the drive spring; and
a retraction collar engaged with the latch mechanism,
wherein the latch mechanism is configured to move from a drive-locked position to a drive-unlocked position relative to the retraction collar;
wherein in the drive-locked position, the retraction collar holds the at least one engagement portion in engagement with the plunger such that extension of the drive spring moves the latch mechanism and retraction collar to expel medicament from a syringe, and
in the drive-unlocked position, the retraction collar does not hold the at least one engagement portion in engagement with the plunger.

In a further aspect, the advancement spring is at least partially received in the interior cavity of the drive spring. In another aspect, the advancement spring is fully received in the interior cavity of the drive spring when the advancement spring is in the compressed condition.

In a further aspect, the activation mechanism is configured to release the drive spring after the advancement spring has expanded distally a predefined distance. Optionally, the predefined distance is between 10 mm and 20 mm, for example 18 mm.

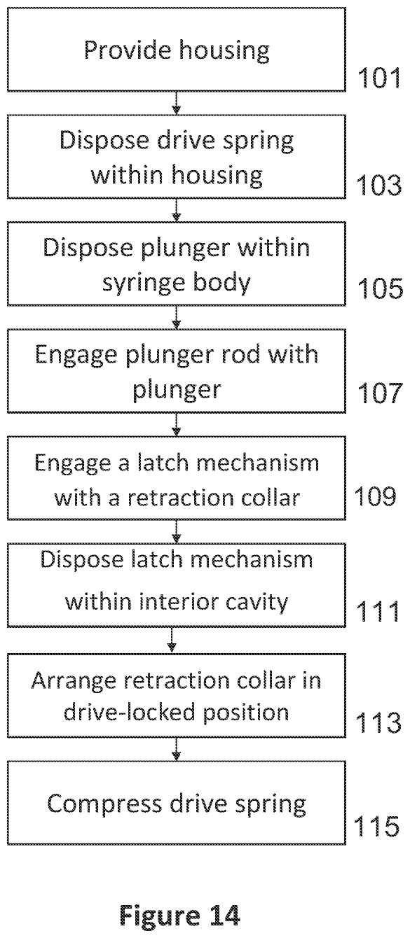

FIG. 14 shows a method of assembling an injection device comprising a power pack as described herein.

At step 101, a housing is provided defining a longitudinal axis. At step 103, a drive spring is disposed within the housing, the drive spring having a distal end and proximal end opposite the distal end along the longitudinal axis, the drive spring defining an interior cavity. At steps 105 and 107, a plunger is disposed at least partially within a medicament container and a plunger rod is engaged with the plunger. At step 109, there is a step of engaging a latch mechanism with a retraction collar to form a drive-lock mechanism, the latch mechanism comprising at least one engagement portion. At step 111, the latch mechanism is disposed at least partially within the interior cavity and releasably engages the at least one engagement portion with the plunger. The step at 113 is arranging the retraction collar in a drive-locked position such that the retraction collar holds the at least one engagement portion in engagement with the plunger and extension of the drive spring moves the latch mechanism and retraction collar distally to expel medicament from a syringe, wherein the retraction collar is configured to move from the drive-locked position to a drive-unlocked position wherein the retraction collar does not hold the at least one engagement portion in engagement with the plunger.

An additional step 115 can include compressing the distal and proximal ends of the drive spring into a compressed condition, and configuring the latch mechanism to retain the drive spring in the compressed condition.

As the reader will appreciate, the steps above can be carried out in any order.

Although an injection device is described above in relation to a first aspect of the disclosure, embodiments may be said to relate to a power pack for an injection device or a power pack for a drive assembly of an injection device. Power packs according to the disclosure may be implemented in an injection device configured to automatically advance a medicament container coupled to a needle into an injection position and to automatically discharge a dose of medicament. More particularly, power packs according to the disclosure may be employed in autoinjectors of the type that advance a medicament container, discharge a dose of medicament, and automatically retract the medicament container relative to the housing after use.

The power packs described herein may be combined with one or more of a damping mechanism, a connection assembly, and a passive safety shield, each of which is described in more detail below.

### Damping Mechanism

The disclosure also provides an example damping mechanism configured to damp a drive assembly of an injection device. A damping mechanism according to the disclosure will be described below in combination with an example drive assembly described above. However, it will be appreciated that the damping mechanism provided by the present disclosure is not limited to use with the drive assembly embodiments described above. Rather, the damping mechanism described below may be implemented in other injection devices having a different drive assembly which nonetheless requires or would benefit from at least part of the drive stroke of the drive assembly being damped.

The injection device of FIGS. 1 and 2 can comprise a damping mechanism configured to damp at least part of the initial stroke of the drive spring when the device is actuated to perform an injection.

In general terms, the damping mechanism comprises a damper configured to frictionally engage a first component of the drive assembly configured to transfer drive from the drive spring to a plunger disposed within a medicament container, as the drive spring moves to deliver an injection to the user. As will be described in more detail below with reference to FIGS. 15-28, the damper and the drive component that it engages (referred to generally in this disclosure as the ‘first drive component’) can take different forms. Although the damper is described in connection with a number of example injection devices herein, it will be appreciated that the damper may be incorporated into other injection devices.

FIG. 15 is an enlarged cross-sectional view of the proximal part of the injection device 1001 shown in FIGS. 1 and 2. In this enlarged view, the drive assembly 1016 from FIG. 1 is shown together with the handle 1003 but in isolation from the rest of the device 1001 shown in FIG. 1.

The drive assembly 1016 (see FIG. 1) comprises a power pack 1030, a housing (henceforth referred to as a proximal housing 1032) and an actuator 1034. The structure and operation of these components is described in more detail with reference to FIGS. 3a-14. However, in summary, the power pack 1030 comprises the drive spring 1017 and the drive-lock mechanism 1036 (described above with reference to FIGS. 3-10), which is configured to couple the drive spring 1017 to the plunger rod 1015. The drive-lock mechanism comprises a latch 1040 configured to engage the plunger rod 1015 and the actuator 1034, and a latch extension 1042 configured to engage the drive spring 1017. The latch extension 1042 is coupled to the latch 1040, which in turn is coupled to the plunger rod 1015. In this way, the drive spring 1017 is configured to transfer drive force to the plunger rod 1015 via the latch 1040 and the latch extension 1042.

As shown in FIG. 15, the drive assembly also includes a damping system configured to damp the initial force of the drive spring when it is released from the storage state shown in FIG. 15. Damping systems according to the present disclosure generally include a damper 1200, which is fixed relative to the housing, and which is configured to frictionally engage a surface of a first drive component configured to move relative to the proximal housing in the longitudinal direction during an injection procedure. In the embodiment described below, the first drive component takes the form of a hollow plunger rod 1015.

The damper 1200 can be fixed within a housing of the device 1001 with a pin 1202. Throughout this disclosure, the housing to which the damper is fixed may be an inner housing of the injection device, such as the proximal housing 1032 of the injection device 1001. Alternatively, the damper 1200 may be fixed to an outer housing such as the handle 1003. The fixation of the damper 1200 within the housing of the device is to ensure that the drive component moves relative to the damper 1200 when the drive spring 1017 is released at the start of an injection procedure.

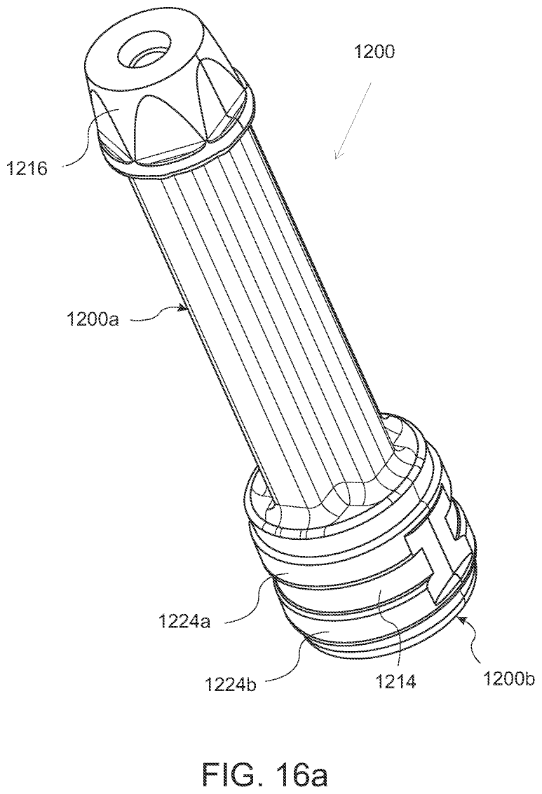

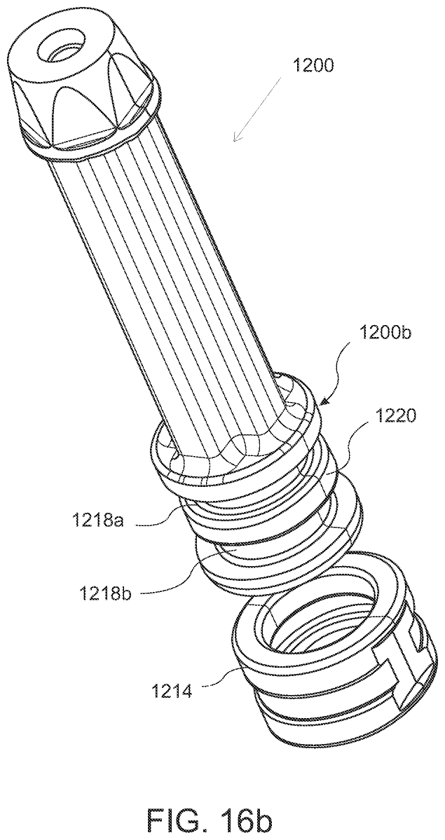

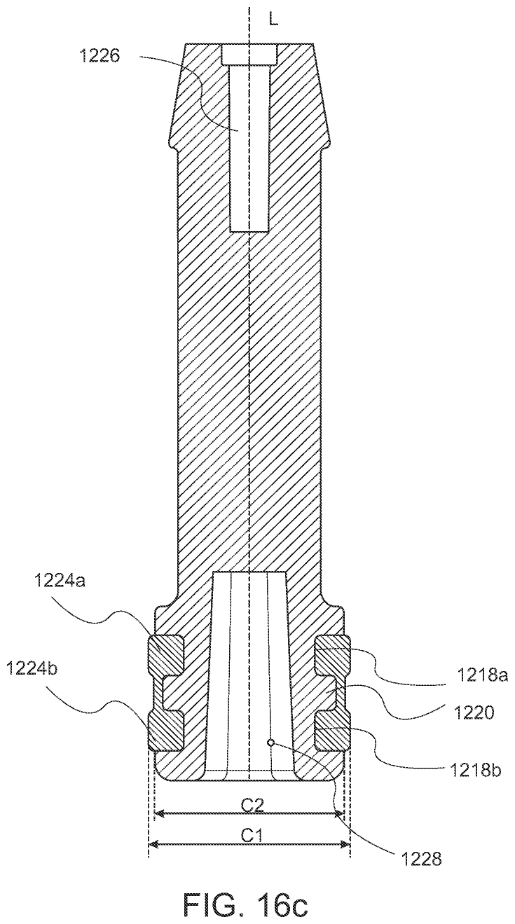

The pin 1202 extends longitudinally along the longitudinal axis L. The pin 1202 includes a head and a shaft. The shaft extends through a hole 1204 in the proximal housing 1032. The head of the pin 1202, when interacting with a shoulder surrounding the hole entrance in proximal housing 1032, provides a stop to secure the pin 1202 longitudinally with respect to the proximal housing 1032. The pin 1202 includes a thread on the shaft, the thread arranged to engage with a threaded hole (shown in FIG. 16c) in the proximal end of the damper 1200 to secure the pin 1202 to the damper 1200 and thus secure the damper 1200 within the housing of the injection device 1. The pin 1202 may be manufactured from plastite or any other suitably rigid material for fixing the damper in place.

Although the embodiment described herein are illustrated with a pin to fix the damper in place, it will be appreciated that the fastener may take other forms than a pin. For example, the fastener may be a flange in the housing for providing a stop for a corresponding part of the damper, an adhesive for adhering the damper to the housing, and/or a mechanical locking arrangement between a part of the damper and a part of the housing. The mechanical locking arrangement may include, for example, a twist-lock or snap-lock arrangement. Alternatively, the damper may be formed integrally with the proximal housing (or the handle) so that a fastener is not required.

The damper 1200 is concentrically arranged within the proximal housing 1032 with the first drive component. The damper 1200 is also concentrically arranged with the latch 1040, the latch extension 1042, and the pin 1202, centred about longitudinal axis L.

With the injection device 1001 in the storage state (as shown in FIG. 15), the damper 1200 is disposed within a channel 1206 formed in the plunger rod 1015. The plunger rod 1015 includes a generally tubular structure that comprises a substantially cylindrical shell with a hollow core. This hollow core provides the channel 1206 in which the damper 1200 is received. The channel 1206 is bounded by an internal wall 1208 and is configured (in size, shape, and position) to receive at least a portion of the damper 1200 via an opening 1210.

In FIG. 15, only a proximal section of the plunger rod 1015 is shown. At the distal end of the illustrated section of the plunger rod 1015, a connecting portion 1212 can be seen, which is configured to connect to a distal section of the plunger rod 1015 (see FIG. 1). Although the plunger rod 1015 shown here comprises multiple components, it will be appreciated that the plunger rod 1015 may comprise a monolithic body, having a hollow bore at its proximal end.

As shown, the damper 1200 comprises a damping member 1214 configured to frictionally engage the inner wall 1208 of the plunger rod 1015 during movement of the plunger rod 1015 relative to the damper 1200. The damping member 1214 has a maximum outer diameter that is larger than a maximum out diameter of the main body of the damper 1200.

Here, the damping member 1214 takes the form of a band or collar of resiliently deformable material which is configured to make contact with at least a portion of the inner wall 1208 of the channel 1206 during an injection procedure. The maximum outer diameter of the damping member 1214 is larger than a minimum inner diameter of the channel 1206. It will be appreciated that the inner diameter of the channel 1206 may be constant along its length (such that the inner diameter of the channel is always less than the outer diameter of the undeformed damping member) or the inner diameter of the channel may vary (such that the inner diameter of the channel is only smaller along part of its length than the outer diameter of the damping member).

By ensuring that at least a portion of the channel 1206 has an inner diameter that is smaller than the outer diameter of the damping member 1214 the damping system can be configured so that the deformable material of the damping member 1214 is compressed against the walls 1208 of the channel 1206 for at least part of the injection process. The compression of the deformable material of the damping member 1214 creates an interference fit between the damper 1200 and the plunger rod 1015 which resists (but does not prevent) movement of the plunger rod 1015 in the proximal direction relative to the damper 1200 under the influence of the drive spring 1017.

By providing a frictional engagement between the damper 1200 and a first drive component (here the plunger rod 1015), the force of the drive spring 1017 can be damped over at least a portion of its travel because the frictional force between the damper 1200 and the plunger rod 1015 acts against the driving force of the spring 1017.

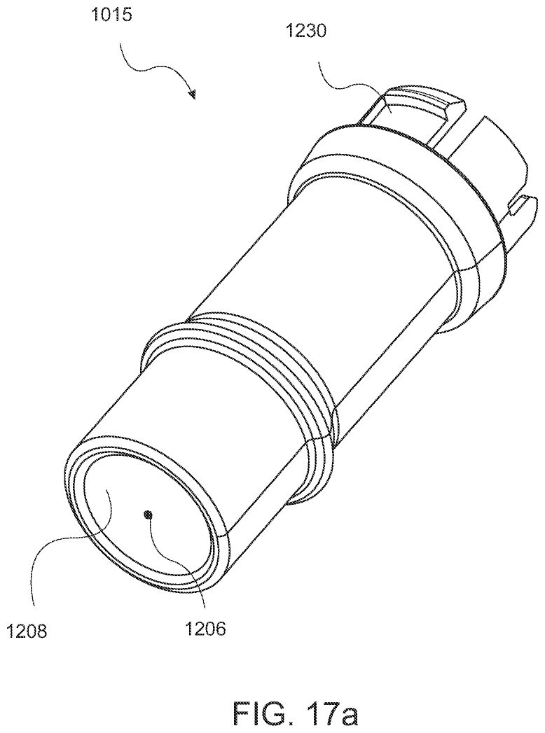

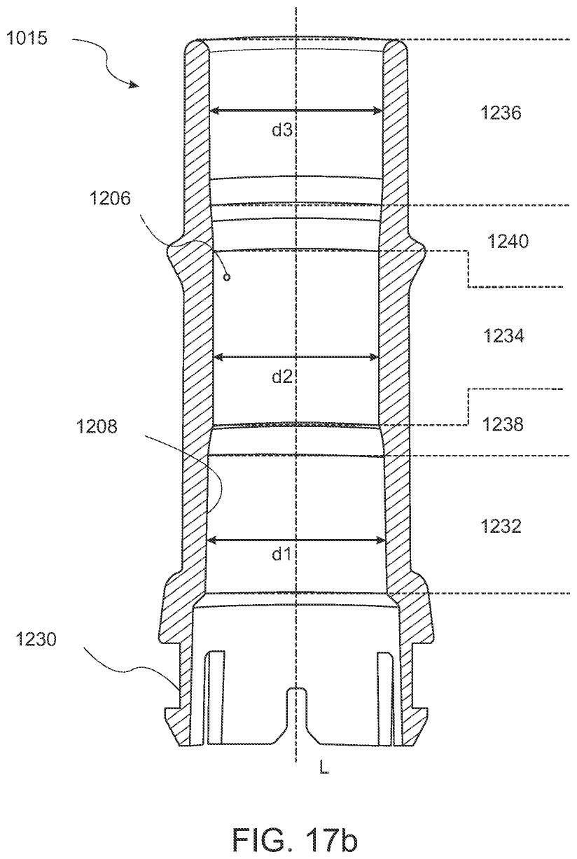

The damper 1200 and the plunger rod 1015 will now be described in more detail with reference to FIGS. 16a-16c and 17a-17b respectively.

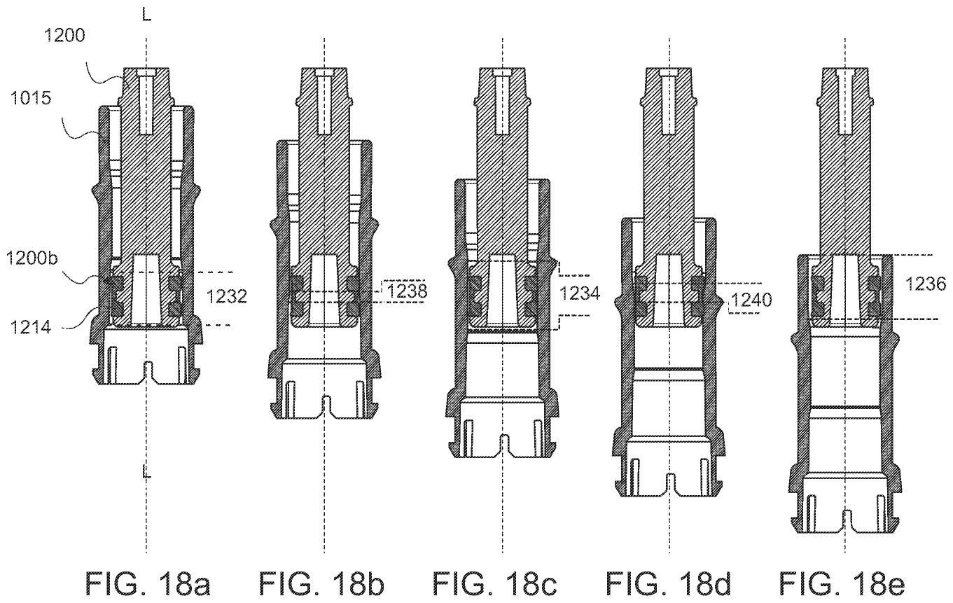

The interaction between the damper 1200 and the plunger rod 1015 along the path of the movement of the plunger rod 1015 will be described in more detail with reference to FIGS. 18a-18e, FIG. 19 and FIG. 19.

FIG. 16A shows an isometric view of the damper 1200 of FIG. 15. FIG. 16B shows an exploded isometric view of the damper 1200 of FIG. 15. FIG. 16C shows a cross section of the damper 1200 in a plane lying along the longitudinal axis L in FIG. 15.

As shown in FIG. 16A and FIG. 16B, the damper 1200 includes an elongate damper main body 1200a and a deformable damping member 1214 arranged to surround a head part 1200b of the damper 1200. In this sense, the damper 1200 may be described as a mandrel. The damper main body 1200a may be manufactured from Nylon resin or another suitable rigid material. The damping member 1214 may be manufactured from silicone or another elastomer or resiliently deformable material.

As shown in FIGS. 16A and 16B, the damper main body 1200a includes a locating part 1216 at a proximal end thereof for locating the proximal end of the damper 1200 in a corresponding channel in the proximal housing 1032. In the illustrated embodiment, the locating part 1216 comprises a hexagonal prism for locating the first end of the damper 1200 in a corresponding hexagonal seat in the proximal housing 1032 in FIG. 15. The locating part 1216 has an annular flange distal to the proximal end of the damper 1200, which is arranged to provide a surface for mating with a shoulder surrounding the seat. The locating part and flange may allow the damper 1200 to be more easily located in the proximal housing 1032 and aligned with the longitudinal axis L during assembly of the injection device 1001.

The head part 1200b is located at or towards a distal end of the damper 1200. The head part 1200b and locating part 1216 are connected by a shaft extending therebetween. As can be seen in FIG. 16B, the head part 1200b is of larger diameter than the shaft elongate main body portion 1200a and locating part 1216.

The head part 1200b of the damper 1200 supports the damping member 1214. Positioning the damping member 1214 at the distal end of the damper 1200 can maximise the travel over which the damping member 1214 engages the inner wall 1208 of the channel 1206 in the plunger rod 1015.

As mentioned before, the damping member 1214 can comprise a band of resiliently deformable material surrounding (or partially surrounding) the head part 1200b of the damper 1200. The damping member 1214 may be overmolded onto the head part 1200b of the damper 1200.

In the configuration shown in FIG. 16B, the head part 1200b includes two circumferential grooves 1218a, 1218b spaced apart along its length, the circumferential grooves 1218a, 1218b forming a circumferential ridge 1220 between them. Though two circumferential grooves 1218a, 1218b are shown, it is contemplated that the head part 1200b can include any number of grooves, such as one groove, three grooves, four grooves, etc.

As shown in FIG. 16A, FIG. 16B and FIG. 16C, the damping member 1214 comprises a ring-shaped collar with an inner profile corresponding to the outer profile of the head part 1200b so as to fit within the circumferential grooves 1218a, 1218b and over the circumferential ridge 1220. This inner profile provides a complimentary portion of the damping member 1214 to engage the head part 1200b to seat the damping member 1214 around the damper 1200. This arrangement may allow the head part 1200b to hold the damping member 1214 in place even when a frictional sheer force acts in either direction parallel to the longitudinal axis L on the damping member 1214. The engagement between the damping member 1214 and the head part 1200b of the damper 1200 can be seen more clearly in FIG. 16C, which shows a cross-sectional view of the damping member seated in the grooves 1218a, 1218b and over the ridge 1220 of the head part 1200b. The inner surface of the damping member 1214 has in inner profile that corresponds to the profile of the grooves and ridges that form the surface of the head part of the damper to ensure this fit.

As shown in FIG. 16B and FIG. 16C, the damping member 1214 includes a groove 1222 therein which extends around the outer circumferential surface thereof. This creates two bands 1224a, 1224b extending circumferentially around the outer surface of the damping member 1214 (shown in FIG. 16A). The bands 1224a, 1224b have a larger outer diameter C1 than the outer diameter C2 of the head part 1200b of the damper 1200. Thus, the bands 1224a, 1224b are the parts of the damper 1200 which are arranged to engage the inner wall 1208 of the plunger rod 1015 as they protrude furthest from the axis of the damper 1200. The damping member 1214 and head part 1200b allow only a portion of the damper 1200 to engage the inner wall 1208 of the channel 1206. This may provide greater control over the friction between the damper 1200 and the plunger rod 1015 by engaging with only a portion of the inner wall 1208 at any one time depending on the position of the plunger rod 1015 in the injection device 1001.

FIG. 16C shows a cross-sectional view of the damper 1200. As shown in FIG. 16C, the damper 1200 comprises a hole 1226. The hole 1226 will be described in more detail below but is configured to receive the pin 1202 (shown in FIG. 15) to fix the damper 1200 within the housing. The damper 1200 may also comprise a socket 1228, optionally a hexagonal socket, arranged to receive a corresponding head of a tool (e.g. a hex Allen wrench) for affixing the damper 1200 to the pin 1202. Although the damper can be fastened to the pin without a socket configured to receive a tool, such an arrangement may be convenient since the position of the damping member and the location of the damper within the elongate proximal housing may make gripping the outer surface of the damper difficult.

In manufacture of the damper of FIG. 16A, FIG. 16B and FIG. 16C, the damping member 1214 can be overmolded onto the head part 1200b. This may help to affix the damping member 1214 to the damper rod 1200a more reliably, by providing improved mechanical grip between the damping member 1214 and the damper rod 1200a compared with other means (e.g. an o-ring in a groove).

This may provide a damper having a more consistent performance, and may lower the cost of manufacture and/or assembly for example by reducing the need for, or complexity of, quality control measures.

In the configuration shown in FIGS. 16a-16c, the grooves 1218a, 1218b and the ridge 1220 extend circumferentially around the head part 1200b to form an unbroken ring. The damping member 1214 comprises a continuous ring with an inner surface that corresponds to the outer profile of the head part of the damper. However, it will be appreciated that this configuration may be modified so that the ring formed by the grooves and/or the ridge is broken and the inner profile of the damping member adapted accordingly.

Moreover, in the illustrated embodiment, the damper comprises two circumferential grooves and a single circumferential ridge formed therebetween. However, the skilled person will appreciate that other configurations are possible. For example, three circumferential grooves may be provided, with a circumferential ridge separating adjacent grooves from each other. Moreover, a single circumferential groove may be provided, in which a portion of the damping member may be seated.

As will be understood, due to the frictional engagement between the plunger rod 1015 and the damper 1200, fixation of the damper 1200 within the housing of the injection device 1001 allows the damper 1200 to act as a brake for the drive spring 1017 as it moves other components relative to the housing. To this end, a fastener is arranged to prevent relative movement between the damper and the housing at least in the direction parallel to the longitudinal axis of the housing.

The plunger rod 1015 shown in FIG. 15 will now be described in more detail with reference to FIGS. 17A-17B. FIG. 17A shows an isometric view of the proximal portion of the plunger rod 1015 of FIG. 15 (the complete plunger rod including the proximal and distal portions is shown in FIG. 1). FIG. 17B is a cross sectional view of the proximal portion of the plunger rod 1015 taken in a plane lying along the longitudinal axis L. As shown in FIG. 17A, the inner wall 1208 defines a channel 1206 that extends through the entire length of the proximal portion of the plunger rod 1015. The distal end of the proximal portion the plunger rod 1015 comprises a snap-fit connection portion 1230 for cooperation with a corresponding snap fit feature on a distal portion of the plunger rod 1015 (shown in FIG. 1) to connect the proximal portion and the distal portion of the plunger rod together.

As shown in FIG. 17B, the channel 1206 has a series of five sections of varying internal diameter. Generally speaking the five sections include a distal section 1232 having a first internal diameter d1, an intermediate section 1234 having a second internal diameter d2, and a proximal section 1236 having a third internal diameter d3. Transition sections 1238, 1240 are provided between each of the sections identified above. A first transition section 1238 connects the distal section 1232 to the intermediate section 1234 and a second transition section 1240 connects the intermediate section 1234 to the proximal section 1236.

As will be appreciated from FIG. 17B, by varying internal diameter of the channel 1206 along its length, the degree of compression of the damping member 1214 can be varied as the plunger rod 1015 advances relative to the damping member 1214, and thereby the damping force provided by the damper 1200 as the injection progresses.

The distal section 1232 is where the head part 1200b of the damper 1200 (comprising the damping member 1214) is positioned when the drive spring 1017 is in the storage stage (shown in FIG. 15). As the plunger rod 1015 advances relative to the damper 1200, the damping member 1214 moves from the distal section 1232, through the intermediate section 1234, into the proximal section 1236.

The inner diameter d2 of the intermediate section 1234 is smaller than the inner diameter d1 of the distal section 1232 and the inner diameter d3 of the proximal section 1236. When the head part 1200b of the damper 1200 is positioned within the intermediate section 1234, the damping member 1214 is compressed. This increase in the compression of the damping member 1214 against the inner wall 1208 of the channel 1206 produces an increase in the normal force between the damping member 1214 and the wall 1208, which in turn increases the friction between these components as the drive spring 1017 acts to move the plunger rod 1015 relative to the damper 1200. When the damping member is located in the distal section 1232 of the channel 1206 (e.g. during storage) or the proximal section 1236 of the channel 1206 (e.g. when the plunger rod 1015 has been advanced to advance the medicament container to an injection position), the damping member 1214 sits in the wider parts of the channel 1206 and is not compressed (or is compressed to a lesser extent) against the walls 1208 of the channel 1206. In this way, the damping force provided by the damping system at the beginning and end of the plunger rod's travel relative to the damper can be eliminated (or reduced). In addition, the distal section 1232 provides a space in which the damping member 1214 remains uncompressed during storage of the injection device and before use. Therefore, the damping performance and reliability of damping of the injection device can be improved compared with an injection device in which the damping member is compressed during storage.

By varying the diameter of the channel section(s) relative to the outer diameter of the damping member, the damping force can be varied as the plunger rod advances relative to the damper. Moreover, the distance over which extension of the drive spring is damped may also be varied by varying the length and/or diameter of the sections described above.

In the embodiment describe above, the smallest internal diameter of the channel is larger than the outer diameter of the rigid head part of the damper. However, at least one section of the channel (here the intermediate section 1234) has an inner diameter equal to or smaller than the outer diameter of the undeformed damping member. By providing at least one section of the plunger rod that has an internal diameter smaller than the outer diameter of the undeformed damping member, a frictional force is created that damps the force of the drive spring.

It will be appreciated that the sections described with reference to FIG. 17B can be modified. For example, in the embodiment of FIG. 17B, only one of the sections (the intermediate section) has an inner diameter smaller than the outer diameter of the damping member. However, multiple sections of the channel may have an inner diameter smaller than the damping member. Moreover, although the embodiment described above includes a channel having five sections, the channel may include more than or fewer than five sections. For example, the channel can have a substantially constant inner diameter along its length, to provide a substantially constant damping force as the plunger rod advances relative to the damper. The transition sections may be omitted, and instead a stepped transition between sections of different diameter may be provided. A plunger rod may also be provided with a channel that tapers smoothly from one end to the other may also be provided. These implementations and others will be apparent to the skilled person in light of this disclosure.

In embodiments, the damper may attenuate the force of the drive spring during the initial travel of the plunger rod. The attenuation of the drive spring force due to the damper may occur prior to and/or during the insertion of the needle. The attenuation of the drive spring force due to the damper may occur during travel of the plunger rod prior to mass flow of the medicinal contents of the medicament container through the needle. Due to fluid back pressure resulting from the restricted flow of the medicinal contents through the needle, attenuation of the drive spring force by the damper may not be necessary during delivery of the medicinal contents through the needle cannula. Therefore, the damper may not necessarily attenuate the drive spring force during the latter stages of operation of the injection device (e.g. following insertion of the needle cannula) to the same degree as in the initial stages of operation.

The interaction between the damper 1200 and the plunger rod 1015 of FIG. 15 as the plunger rod 1015 advances relative to the damper 1200 will now be described in more detail with reference to FIG. 18A-18E.

FIG. 18A-18E are cross-sectional views of the plunger rod 1015 and damper 1200 in a plane lying along the longitudinal axis L. FIG. 18A shows the damper 1200 with the head part 1200b seated in the distal section 1232 of the channel 1206. FIGS. 18b-18d show the damper 1200 within the channel 1206 as the plunger rod 1015 advances during an injection process to its distalmost position relative to the damper.

As shown in FIG. 18A, before initiation of an injection (by release of the drive spring), the head part 1200b of the damper 1200 comprising the damping member 104 is located in the distal section 1232 of the channel 1206. As described above, the inner diameter of this section is larger than the outer diameter of the damping member 1214 and so the damping member 104 does not contact (or is not compressed against) the inner walls 1208 of the channel 1206 (see FIGS. 17a and 17b). The result of this arrangement is a relatively low (or absent) frictional force between the plunger rod and the damper due to the limited (or non-existent) engagement of the damping member with the inner wall of the channel.

FIG. 18B shows the relative position of the plunger rod 1015 relative to the damper 1200 after the head part 1200b of the damper 1200 has moved partially into the intermediate section 1234 of the channel 1206, bridging the first transitional section 1238. Here, because the inner diameter of the channel 1206 is smaller than the outer diameter of the damping member 1214 (in the undeformed state), the proximal portion of the damping member 1214 is compressed against the inner walls 1208 of the channel 1206. This distal portion of the damping member 1214 still occupies the distal section of the channel 1206 and so is not compressed (or is compressed to a lesser extent than the proximal portion) against the inner wall of the channel. The result of this is an intermediate frictional force between the plunger rod 1015 and the damping member 1214 due to the increasing engagement of the damping member 1214 with the inner wall of the channel 1206.

FIG. 18C shows the relative position of the damper 1200 and the plunger rod 1015 of FIG. 15 when the plunger rod 1015 has advanced yet further relative to the damper 1200 such that the head part 1200b of the damper 1200 is located in the intermediate section 1234 of the channel 1206. Here, along its length, the damping member 1214 is compressed against the inner wall 1208 of the intermediate section 1234 of the channel 1206. The result of this is a relatively high frictional force between the plunger rod 1015 and the damping member 1214 due to the engagement of the damping member 1214 with the inner wall of the channel 1206.

As shown in FIG. 18D, as the plunger rod 1015 continues its distal travel relative to the damper 1200, the head part 1200b of the damper 1200 is disposed partially within the intermediate section 1234 (in which the damping member is compressed) and partially in the proximal section 1236 of the channel, in which the damping member is not compressed (or is compressed to a lesser extent), bridging the second transition section 1240. In this position, like the position shown in FIG. 18B, only a portion of the damping member 1214 is compressed since only the distal portion of the damping member is still disposed within the narrower intermediate section 1234. The result of this is again an intermediate frictional force between the plunger rod 1015 and the damping member 1214 due to the engagement of a portion of the damping member 1214 with the inner wall of the channel 1206.

Turning finally to FIG. 18E, when the plunger rod 1015 reaches its distalmost point of travel relative to the damper 1200, the head part 1200b of the damper 1200 is located in the proximal section 1236 of the channel 1206. Here, because the inner diameter of the proximal section 1236 of the channel 1206 is larger than the outer diameter of the damping member 1214, the damping member 1214 is not compressed against the inner wall 1208 of the channel 1206. The result of this is a relatively low (or absent) frictional force between the plunger rod 1015 and the damping member 1214 due to the limited (or non-existent) engagement of the damping member 1214 with the inner wall of the channel 1206.

In light of the above, the distal section 1232 of the channel 1206 may be described as a ‘damper storage zone’ in which the damping member 1214 will be in an uncompressed state when it is positioned therein (see FIG. 18A). This position of the damper 1200 relative to the plunger rod 1015 corresponds to the storage state of the device, in which the drive spring 1017 is compressed, before an injection has been initiated. Adapting the distal section 1232 of the channel 1206 to receive the head part 1200b of the damper with little to no engagement between the damper and the plunger rod may be useful because the damping mechanism can be assembled by inserting the damper into the distal end of the plunger rod, without needing to overcome significant frictional engagement between the damping member and the plunger rod to assemble the device.

The intermediate section 1234 may be described as a ‘damper compression zone’ in which the damping member 1214 will be in a compressed state when it is positioned therein. During this stage, the damping member 1214 will be under maximum compression, thus damping the force of the drive spring 1017 immediately after the device is actuated. This may minimize impact of the medicament container on the internal components of the injection device. Alternatively, or in addition, this may avoid a high impact which may startle user or avoid a jolt that may cause unexpected user errors.

The proximal section 1236 may be described as an ‘undamped zone’ in which the damping member 1214 is mainly or entirely released from its compressed state when it is positioned therein. Providing an undamped zone at the proximal end of the channel may be useful, because it allows the force of the drive spring to be damped over only a portion of its travel (e.g. the initial portion of the travel), in which the drive force of the drive spring is highest, and whilst the medicament container is being advanced into the injection position. Moreover, the wider proximal section (with the optional tapered transition zone between the proximal section and the intermediate section) can facilitate retraction of the plunger rod 1015 relative to the damper 1200 after an injection is completed.

Although the plunger rod shown in FIGS. 15-18e comprises a channel having a variable inner diameter, it will be appreciated that the channel may have a substantially constant inner diameter, in which the damping member engages the inner wall of the channel throughout substantially the entire length of travel of the plunger rod relative to the damper. An example of such an embodiment is not shown the drawings, but it will be appreciated that the plunger rod shown in FIGS. 18a-18e may simply replaced by a plunger rod comprises a substantially cylindrical bore having a constant inner diameter.

Other configurations in which a damper is received within a hollow plunger rod are also disclosed, as will be described below with reference to FIG. 19.

FIG. 19 shows a cross-sectional view of a further embodiment of the present disclosure. The embodiment shown in FIG. 19 is similar to the embodiments as described above. As can be seen in 19, a device 4001 comprises a drive assembly 4016, which includes a proximal housing 4032 that houses a drive spring 4017, a latch 4040, a latch extension 4042, and an actuator 4034. Like the embodiment described above, the latch 4040 and the latch extension 4042 cooperate to deliver drive force from the drive spring 4017 to a plunger rod 4015 that comprises a channel 4206 or hollow portion at its proximal end.

In contrast with the embodiment described with reference to FIG. 15, the first drive component 4015 in the embodiment of FIG. 19 has two sections of different internal diameter that are arranged to receive a damper 2400 in various states of extension of the plunger rod 4015: a distal section 4232 first inner diameter and a proximal section 4236 having a second inner diameter that is larger than the first inner diameter.

The damper 4200 of FIG. 19 also differs from the damper 1200 of FIG. 15. Whereas the damper 1200 of FIG. 15 includes a single damping member 1214 having an internal profile configured to mate with an external profile of the head part 1200b of the damper 1200, the damper 4200 of FIG. 19 comprises a head part 4200b that include a plurality of annular grooves, each being configured to receive an O-ring therein. The damper 4200 also includes a main body part 4200a supporting the head part 4200b.

The head part 4200b of damper 4200 shown in FIG. 19 has three circumferential grooves 4218a, 4218b, 4218c. Three separate damping members 4214a, 4214b, 4214c (here, each taking the form of an O-ring) are seated in a corresponding one of the circumferential grooves 4218a, 4218b, 4218c on the head part 4200b. When in place on the head part 4200b, the damping members 4214a, 4214b, 4214c have an outer diameter which is greater than the outer diameter of the head part 4200b.

The operation of the damper 4200 of FIG. 19 is similar to the operation of the damper 1200 of FIG. 15.

In the position shown in FIG. 19, the damping members 4214a, 4214b, 4214c of the damper 4200 are compressed against the inner wall 4208 of the channel 4206 because the inner diameter of the distal section 4232 of the channel 4206 is smaller than the outer diameter of the damping members 4214a, 4214b, 4214c. The result is a relatively high frictional force between the plunger rod and the damper due to the engagement of the damping member with the inner wall of the channel.

When the damping members are disposed in the proximal section 4236 of the channel 4206, the larger diameter of the channel no longer compresses the damping members (or compresses them to a lesser extent). The result is a relatively low (or absent) frictional force between the plunger rod and the damper due to the limited (or non-existent) engagement of the damping member with the inner wall of the channel.

A feature of the plunger rod 4015 of FIG. 19 which is not present in the embodiment of FIG. 15 is a stop 4242 formed as a cap to close the channel 4206 at the distal end of the distal section 4232. The stop 4242 forms a surface for the end of the head part 4200b of the damper 4200 to abut during assembly of the device and prevents the first drive component 4015 from being retracted too far at the end of the injection cycle when the first drive component 4015 is returned to its original position. It will be appreciated that the stop can be omitted from this embodiment or added to the embodiment of FIG. 15 as desired. The stop 4242 may help to prevent the damper 4200 from interfering with other components of the injection device, for example a component such as a PCB positioned between the damper and the plunger.

It will be appreciated that the damper and plunger rod are not limited the types described with reference to FIGS. 15 and 19, in which a damper engages the inner walls of a channel formed in the plunger rod. Rather, according to the disclosure, the damper can be configured to engage other drive components.

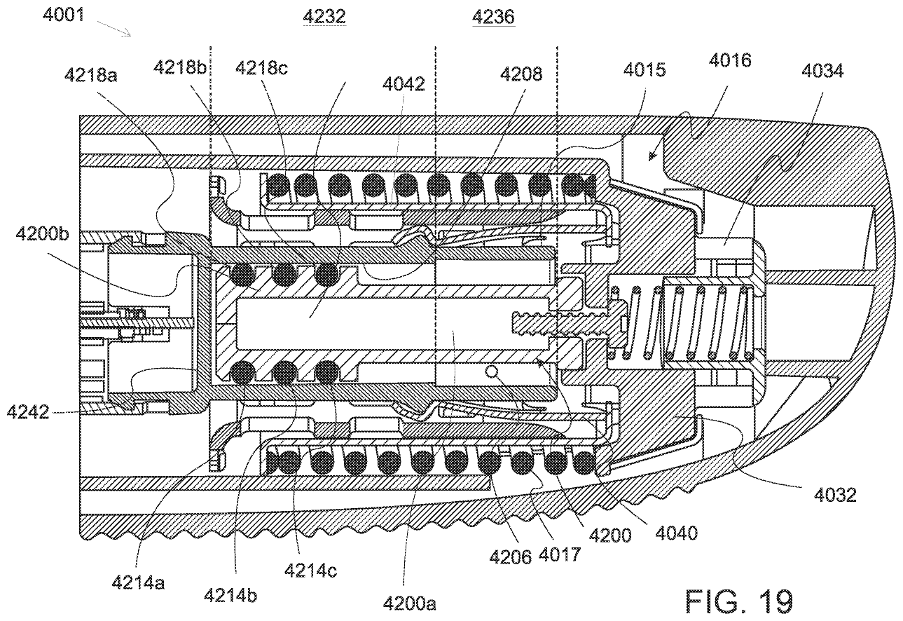

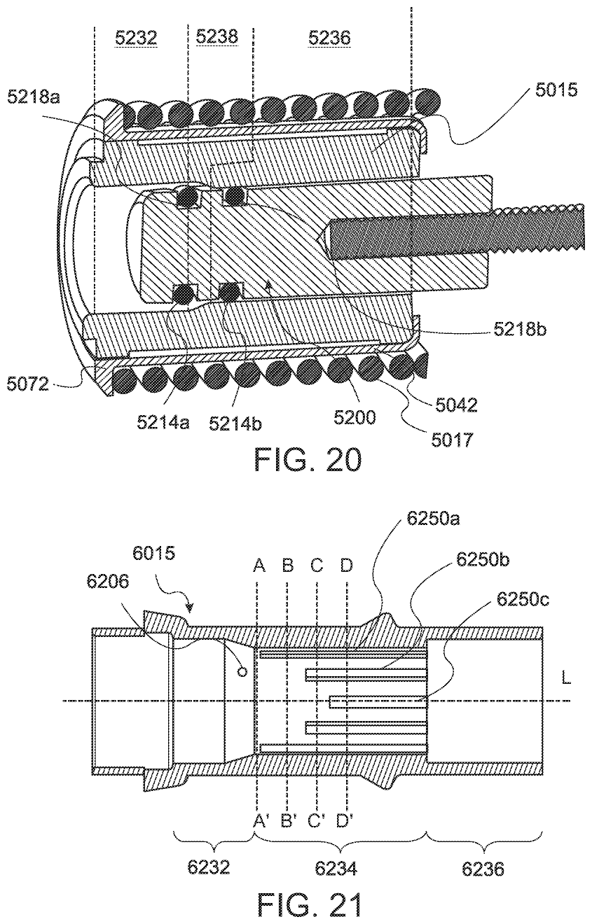

The power pack is also not limited to that shown in FIGS. 15 and 19 in that a feature similar to the latch extension may transmit the force of the drive spring directly to the first drive component without the use of a latch. These modifications and others will become apparent from the following discussion of FIGS. 20 to 28e.

FIG. 20 shows a cross-sectional view of an embodiment in which a damper 5200 is configured to directly engage a component of the drive assembly (rather than the plunger rod). FIG. 20 shows a similar arrangement to the arrangement shown in FIGS. 15 and 19 in that the drive assembly comprises a drive spring 5017 and a damper 5200 configured to damp at least the initial expansion of the drive spring 5017.

In contrast with the embodiment described with reference to FIG. 15, the damper 5200 in the embodiment of FIG. 20 does not have an enlarged head part.

In more detail, the main body of the damper 5200 shown in FIG. 20 is a rod of substantially uniform diameter along substantially its entire length notwithstanding two circumferential grooves 5218a, 5218b around the distal end of the damper 5200. The circumferential grooves 5218a, 5218b each receive a corresponding one of two damping members 5214a, 5214b. The damping members here take the same form as the O-ring damping members described with reference to FIG. 19.

In contrast with the embodiment described with reference to FIG. 15, the drive component that the damper engages is a sleeve 5015 coupled to the drive spring 5017. Accordingly, the sleeve 5015 acts as the first drive component that is driven by the drive spring 5017. The sleeve 5015 may form part of the plunger rod of a device.

The sleeve 5015 comprises a channel, somewhat similar to the channels of the plunger rods described above, in that it is configured to receive the damper, and its inner walls are configured to frictionally engage the damping member 5200.

As can be seen in FIG. 20, the channel of the sleeve 5015 has three sections: a distal section 5232 having a first diameter, a proximal section 5236 having a diameter (the first diameter being larger than the second diameter) and a transition section 5238 which provides a tapered transition between the distal section and the proximal section. Like the embodiments described above, the sleeve 5015 comprises at least one section that has an inner diameter that is smaller than an outer diameter of the damping members 5214a, 5214b. In the illustrated embodiment, the distal section 5232 has an inner diameter that is larger than the outer diameter of the damping members 5214a, 5214b. Thus, the distal section 5232 may be described as a damper storage zone. The proximal section 5236 has an inner diameter that is smaller than the outer diameter of the damping members 5214a, 5214b and so the proximal section 5236 may be described as a damper compression zone.

Unlike the first drive components shown in FIGS. 15 and 19, the damped drive component (here the sleeve 5015) is not coupled to the drive spring 5017 via a latch mechanism. Instead, the first drive component is fitted within drive sleeve 5042 that has a similar form to latch extension 5042 described above. The drive sleeve 5042 (which is similar in shape to latch extension 1042) comprises a distal flange 5072 against which the drive spring 5017 bears. The sleeve 5015 is fitted within the drive sleeve 5042, so that distal movement of the drive sleeve 5042 under the influence of drive spring 5017 acts to move the drive sleeve 5042 and the sleeve 5015 together in the distal direction.

The sleeve 5015 and/or the drive sleeve 5042 may be configured to transmit this drive force to a plunger rod (not shown) which is configured to drive a plunger distally within a medicament container to expel a dose of medicament. The distal end of the sleeve 5015 may therefore include locating features (e.g. a raised annular flange) to engage corresponding features on a proximal end of a plunger rod.

The embodiment described above includes a channel in which the inner diameter of the channel varies along the length of the channel. In this way, the compression of the damping member (and thus the damping force) can be varied as the expansion of the drive spring progresses. It will however be appreciated that the inner diameter of the channel can be constant along its length, such that the damping force remains substantially constant as the channel moves distally relative to the damper.

Turning now to FIG. 21, yet another embodiment of the disclosure will be described. FIG. 21 shows a plunger rod 6015 that comprises a plurality of grooves 6250a, 6250b, 6250c extending in a generally longitudinal direction. The plurality of grooves 6250a, 6250b, 6250c can extend along the longitudinal axis or parallel thereto. As will be explained in more detail below, the grooves 6250a, 6250b, 6250c can be provided to reduce the surface area of the channel wall that the damping member is in contact with, and provides space into which a deformable damping member can deform to reduce the compressive force applied to the damping member by the walls of the channel. This may reduce the magnitude of frictional force between the first drive component and the damper in an area of the first drive component engaged by the damper.

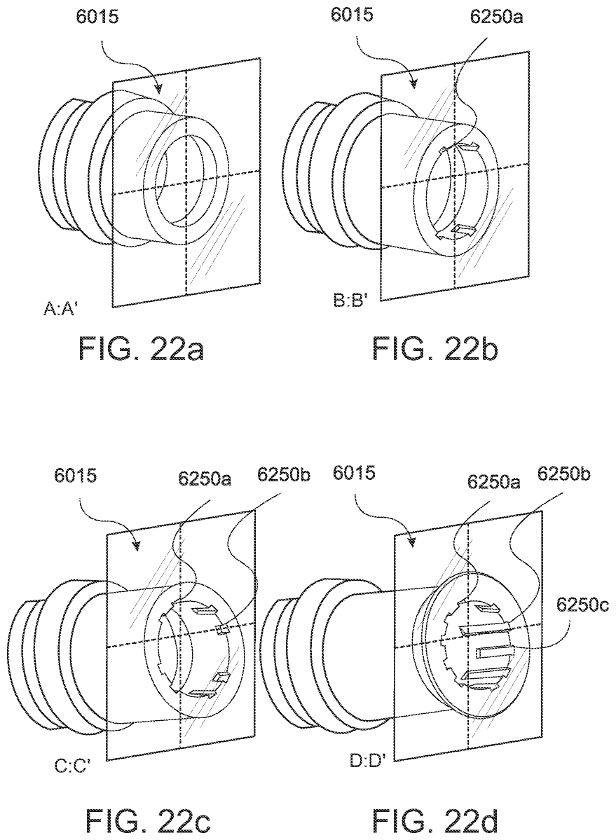

More specifically, FIG. 21 shows a cross sectional view of a modified version of the plunger rod 7015 of FIGS. 17a and 17b. FIGS. 22A, 22B, 22C and 22D, show cross sections through planes A:A′, B:B′, C:C′, and D:D′, respectively, of FIG. 21.

FIG. 21 shows essentially the same first drive component as shown in FIGS. 17a and 17b, but for the following differences. As seen in FIG. 21, the intermediate section 6234 of the channel 6206, which is positioned between the distal section 6232 and the proximal section 6236, has a series of grooves 6250a, 6250b, 6250c included in the inner surface thereof. The grooves 6250a, 6250b, 6250c extend parallel to the longitudinal axis L of the plunger rod 6015. All of the grooves 6250a, 6250b, 6250c begin at the proximal end of the intermediate section 6234 and extend longitudinally from the proximal end toward the distal end. A first groove 6250a extends along almost the entirety of the intermediate section 6234, stopping short of the distal end of the intermediate section 6234. A second groove 6250b is circumferentially spaced from the first groove 6250a and extends along approximately two thirds of the length of the intermediate section 6234. A third groove 6250c is circumferentially spaced apart from both the first and second grooves 6250a, 6250b and extends along approximately half of the length of the intermediate section 6234. The first, second and third grooves 6250a, 6250b, 6250c are repeated in the following sequence around the circumference of the intermediate section 6234: first, second, third, second, first, first, second, third, second, first.

As shown in FIG. 22A, a first cross section A:A′ taken through the plunger rod 6015 of FIG. 21 between the distal end of the intermediate section 6234 and the end of the first grooves 6250a includes none of the grooves 6250a, 6250b, 6250c. As shown in FIG. 22B, a second cross section B:B′ taken through the plunger rod 6015 of FIG. 21 between the end of the first groove 6250a and the end of the second groove 6250b includes only the first grooves 6250a. As shown in FIG. 22C, a third cross section C:C′ taken through the plunger rod 6015 of FIG. 21 between the end of the second groove 6250a and the end of the third groove 6250c includes only the first grooves 6250a and second grooves 6250b. As shown in FIG. 22D, a fourth cross section C:C′ taken through the plunger rod 6015 of FIG. 21 between the end of the third groove 6250a and the proximal end of the intermediate section 6234 includes all of the first, second and third grooves 6250a, 6250b, 6250c.

Thus, the number of grooves 6250a, 6250b, 6250c per unit circumferential length of the intermediate section 6234 increases with distance from the distal to the proximal end of the intermediate section 6234. Thus, within the intermediate section 6234, the surface area in contact with the damping member(s) of the damper reduces as the plunger rod 6015 advances and the damping members move from the distal end to the proximal end of the intermediate section 6234. This in turn leads to a stepwise reduction in friction force between the damper and the plunger rod 6015 as the plunger rod 6015 advances and the damper engages with a smaller and smaller contact area of the inner wall of the plunger rod 6015. In other words, a percentage of the inner wall comprised by the grooves may decrease in a distal direction.

Although the embodiment shown in FIG. 21 comprises ten grooves in an intermediate portion. However, it will be appreciated that the number of grooves, and the number of different grooves having a different length can be varied. Moreover, even though the grooves are described here in the context of the drive element having a variable inner diameter along its length, the grooves described herein may be used as an alternative to this arrangement.

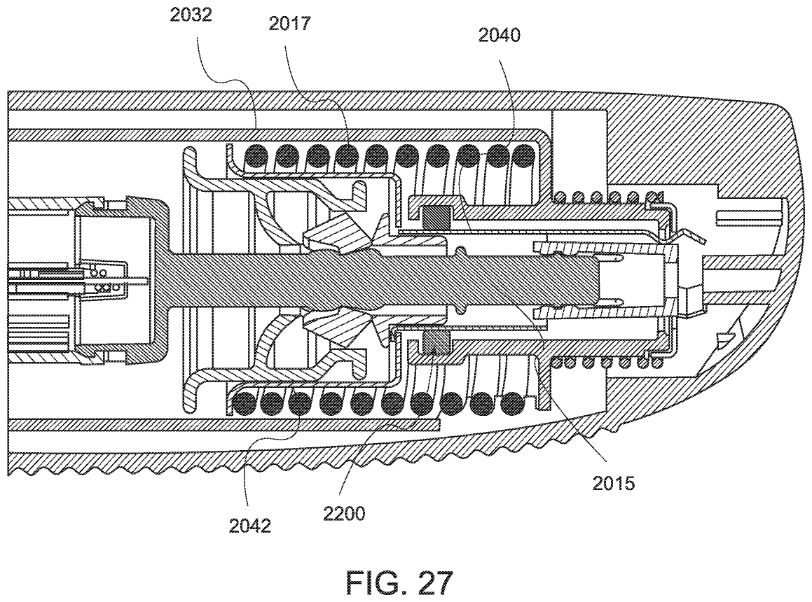

Although this embodiment is described with reference to a plunger rod, it will be appreciated that the grooves described above may be incorporated into the sleeve 5015 of FIG. 20 or another drive component configured to move relative to a damper. Moreover, although in the foregoing description the grooves are in an inner wall of the plunger rod, embodiments are not limited thereto. For example, where the damper is annular (see e.g. FIG. 27), and the first drive component is arranged inside the damper as described in following sections of this disclosure, the grooves may be formed on an outer circumferential surface of the first drive component arranged to engage the damper. Alternatively, or in addition, the grooves may be on the portion of the damper which is arranged to engage the first drive component.

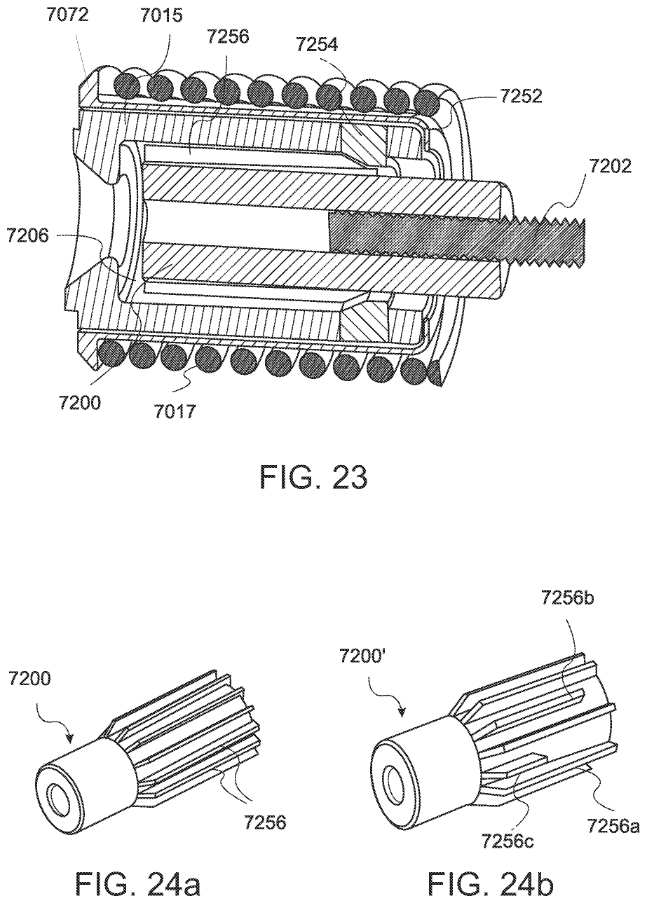

Turning now to FIGS. 23-24b, damping systems according to the disclosure can include a damper comprising a plurality of deformable splines (or ridges) extending in a longitudinal direction thereof (i.e. substantially parallel to the longitudinal axis of the injection device), the splines being arranged to be compressed by a first drive component, for example a plunger rod of another component of the drive assembly. The first drive component can comprise a compression ring configured to compress the splines. As will be understood from this disclosure, the magnitude of the frictional force between the damper and the first drive component can be controlled using the splines on the portion of the damper which is arranged to engage the first drive component. Like the grooves described above, the splines can be varied in length, width or thickness, or the number of splines may be varied per unit circumferential length, to reduce or increase the friction between the damper and the first drive component.

In more detail, as shown in FIG. 23, some embodiments comprise a damper 7200 arranged concentrically with a first drive component, which here takes the form of a sleeve 7015 similar to the sleeve 5015 of FIG. 19. Like the embodiment shown in FIG. 19, the sleeve 7015 is coupled to a drive sleeve 7252, which provides a distal flange 7072 against which the drive spring 7017 can bear. Also like the embodiment shown in FIG. 20, the damper 7200 is fixed within the housing with a pin 7202, which is threadedly engaged with the damper.

As shown in FIG. 23, the sleeve 7015 includes a channel 7206 configured to receive at least a portion of the damper 7200. The sleeve 7015 also comprises a compression ring 7254, at or near the proximal end of the channel 7206. The compression ring 7254 is made from a material (e.g. a metal) which will not deform under the force exerted on it by the damper 7200 as the sleeve 7015 advances relative to the damper 7200 during an injection process. The compression ring 7254 is located toward a proximal end of the sleeve 7015 and occupies only a fraction of the length of the first sleeve 7015.

The damper 7200 comprises a plurality of splines 7256 on its outer surface, extending in a longitudinal direction. The splines 7256 extend longitudinally along a portion of the main body of the damper, at the distal end of the damper 7200. The splines 7256 protrude radially from the circumferential surface of the main body of the damper and are spaced apart from each other around the outer circumference of the main body of the damper. The splines 7256 extend outwardly to give the damper 7200 a greater diameter than the cylindrical rod at the distal end of the damper 7200. This diameter (the ‘spline diameter’) is also greater than the minimum internal diameter of the compression ring 7254.

The splines have a substantially constant height along their length, apart from at their proximal end, where they taper towards the main body of the damper 7200. The outer diameter of the splines 7256 is larger than a minimum inner diameter of the compression ring 7254. At least the splines of the damper are formed of a resiliently deformable material so that the splines can be compressed against the inner surface of the compression ring. The splines may be made from an elastomer, such as a thermoplastic elastomer.

As shown in FIG. 23, the compression ring 7254 tapers at one end from a diameter which is greater than the spline diameter to a diameter which is less than the spline diameter. Thus, the compression ring partitions the channel 7206 into sections such that there is a damper storage zone in the section of the channel on the distal side of the compression ring 7254, a damper compression zone formed by compression ring 7254, and an undampened zone in the section on the proximal side of the compression ring 7254. The channel 7206 has (notwithstanding the compression ring 7254) a constant internal diameter which is greater than the outer diameter of the splines 7256 that extend along the body of the damper 7200. The splines 7256 of FIG. 23 can be seen clearly in FIG. 24A, which shows a perspective view of the damper of FIG. 23.

Referring still to FIG. 23, when the device is in the storage state (with the drive spring is fully compressed), the sleeve 7015 is in the fully retracted position relative to the damper 7200, and the distal damper storage zone houses the portion of the damper 7200 having the splines 7256. As the outer diameter of the splines 7256 is less than the inner diameter of the sleeve 7015 and the outer diameter of the main body of the damper 7200 is less that the minimum inner diameter of the compression ring 7254, there is no engagement between the damper 7200 and the inner walls of the first drive component 7015.

In operation, as the first drive component 7015 advances relative to the damper 7200 under the driving force of the drive spring 7017, the splines 7256 are forced through the compression ring 7254. Since the outer diameter of the splines 7256 is less than the minimum inner diameter of the compression ring 7254, and the splines 7256 are resiliently deformable, the splines 7256 engage the compression ring 7254 and a normal force due to the deformation of the splines 7256 is exerted by the splines on the compression ring 7254. Thus, a frictional force is generated between the damper 7200 and the sleeve 7015. As with other embodiments, this frictional force acts to attenuate the force exerted by the drive spring 7017 on the plunger and/or medicament container.

The splines can take different forms, as will now be described with reference to FIGS. 24a and 24b. As mentioned above, FIG. 24A shows a perspective view of the damper 7200 of FIG. 23. As can be seen in this figure, the splines 7256 of the damper 7200 are of equal length, starting and ending at the same point, circumferentially distributed around the main body of the damper 7200. This arrangement provide substantially constant damping force as the splined portion of the damper 7200 passes through the compression ring 7254.

FIG. 24B shows an alternative embodiment in which a damper 7200′ comprises splines 7256a′, 7256b′, 7256c′ of varying length. Varying the length of the splines in the manner shown in FIG. 24B can vary the damping force provided by the damping system as the sleeve 7015 comprising the compression ring 7254 advances relative to the damper 7200.

The splines shown in FIG. 24A each extend approximately the same distance in the proximal direction from the distal end of the damper. The frictional force between the compression ring and the damper is therefore substantially constant throughout the travel of the drive element relative to the damper. However, in the same way that grooves of varying length can be used to vary the frictional engagement between the damper and the plunger rod 6015 in the embodiment of FIG. 21, in the embodiment shown in FIG. 24B, the length of the splines is varied to vary the frictional engagement between the damper and the first drive element as the first drive element advances relative to the damper.

More particularly, FIG. 24B shows a damper 7200′ having a first spline 7256a which extends along the full length of a distal portion of the damper 7200′. A second spline 7256b extends along approximately three quarters of the distal portion so that it stops short of the distal end of the damper 7200′ by approximately one quarter of the length of the distal portion. A third spline 7256c extends along approximately one third of the length of the distal portion so that it also stops short of the distal end of the damper 7200′ by approximately two thirds of the length of the distal portion.

In general terms, the splines may be arranged so that the contact area between the splines and the compression ring varies as the damper passes through the compression ring. In embodiments, the plurality of splines comprises at least one first spline and at least one second spline each extending along a portion of the damper, wherein the first and second splines are different lengths (the length being the dimension measured parallel to the longitudinal axis L of the damper and the injection device). Alternatively, or in addition, the width of at least one spline may vary along its length. Alternatively, or in addition, the aforementioned ‘spline diameter’ may vary with position along the length of the damper.

The differing lengths of the splines provides a different density of splines with distance along the longitudinal direction of the damper and therefore a changing contact area per unit length. This may vary the damping force as first drive component advances from the fully retracted position to an extended position. The denser the splines along any portion of the damper, the greater the damping force due to the greater contact area between the damper and first drive component. In other words, a percentage of the outer surface of the of the damper comprising splines may decrease in a distal direction. Conversely, using equal lengths of splines may provide uniform damping along the length of the damper.

In the any of the above described dampers, the splines themselves may be of constant width (measured in a circumferential direction of the damper around the longitudinal axis of the injection device). Alternatively, one or more of the splines may have a width that varies along its length. The width may vary gradually so as to taper along the length of the spline, or there may be step changes in the width along the length of the spline. This is further way to adjust the contact area between the damper and first drive component along the length of the damper.

The spline diameter of the damper may be uniform along the length of the damper. Alternatively, the spline diameter may vary along the length of the damper. The spline diameter may vary gradually so as to taper along the length of the damper, or there may be step changes in the spline diameter along the length of the spline. A varying spline diameter may vary the normal force between the damper and the first drive component as the splines pass through the compression ring. Thus, the friction force and hence the attenuation of the drive spring force may vary as the first drive component advances.

Although the above described splines and grooves are described above as being arranged parallel to the longitudinal axis of the damper, this disclosure is not limited thereto. For example, the grooves or splines may be arranged helically around the surface of the respective component. In this arrangement, the properties (i.e. width, length, radial position, or density) of the splines or grooves may be varied as a function of longitudinal position in similar ways to those described in the foregoing. Helical splines or grooves may vary the contact area and/or normal force between the damper and first drive component to create the desired friction force profile. However, a first drive component having longitudinal splines may be simpler to manufacture.

Moreover, although the embodiments above include a splined damper configured to interact with a sleeve that forms part of the drive assembly, it will be appreciate that a compression ring may also be provided in a portion of a plunger rod, such as the plunger rod 1015 of FIG. 15.

In yet further embodiments of the disclosure, a damping system may be provided which is configured to vary the frictional force between a first drive element and a damper by varying the compressive force exerted by a tube on a fixed damper by varying the thickness of the tube walls along the length of the tube. Although the structure of this embodiment is somewhat different to the embodiments described above, the underlying principle (in which a variable compressive force is applied between a damper and a drive component) is similar, as will be explained below. In general terms, in these embodiments the first drive component can take the form of an auxiliary drive member including a compression sleeve. The compression sleeve has a constant internal diameter, but a thickness of the sleeve wall varies along its length. The compression sleeve is integrally formed with or coupled to a drive component that is configured to advance distally under the influence of the drive spring. The damper is arranged inside the sleeve and engages the inner surface of the sleeve.

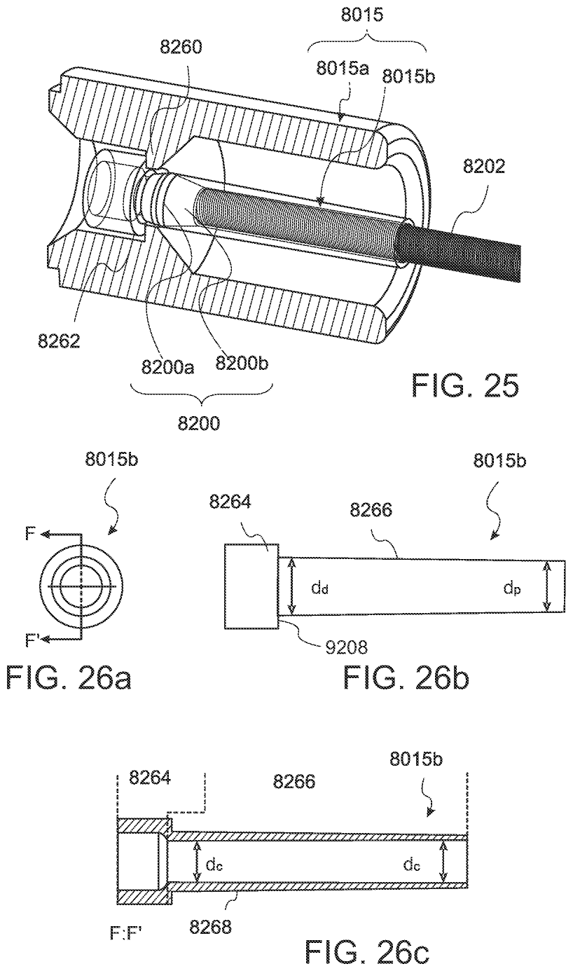

FIG. 25 is an isometric cross sectional of a view of a damping system comprising a first drive component 8015 formed from drive body 8015a and compression sleeve 8015b, a damper 8200 and pin 8202.

The damper 8200 includes a damper main body 8200a and a ferrule 8200b. The damper main body 8200a is formed as a body having a tapered external diameter toward each end and a threaded hole formed therethrough for receiving the pin 8202. The maximum external diameter of the damper main body 8200a is formed between the two tapered ends and is larger than the outer diameter of the pin 8202. The ferrule 8200b is a ring-shaped member and is formed on this outer part of the damper main body 8200a. The ferrule 8200b may attenuate the force of the drive spring by plastically deforming upon entry into the compression sleeve 8015b. The ferrule may be formed from metal, an injection moulded plastic or another plastically deformable (or substantially non-elastomeric) material. The ferrule 8200b may have a hardness and strength which are greater than those of the compression sleeve 8015b.

The ferrule 8200b surrounds the damper main body 8200a and has a larger outer diameter than the outer diameter of the main body of the damper 8200 to provide a contact surface for contacting the inner walls of the drive component in a similar way to the damping member of other embodiments described in this disclosure. Although the ferrule 8200b is mounted on the main body 8200a in the illustrated embodiment, it will be appreciated that the damper main body may be omitted and the ferrule may be mounted directly on the pin 8202, as long as the outer diameter of the ferrule is larger than the outer diameter of the pin.

Referring still to FIG. 25, the first drive component 8015 includes a main drive body 8015a and compression sleeve 8015b. The main drive body 8015a has a generally cylindrical body that comprises a collar 8260 that forms a distal-facing shoulder that a corresponding shoulder 8262 of the compression sleeve 8015b may bear against. The abutment of these shoulders prevent distal movement of the drive body 8015a relative to the compression sleeve 8015b, and ensures that the drive body 8015a and the compression sleeve 8015b move together (at least in the distal direction) under the influence of the drive spring.

The compression sleeve 8015b extends proximally from the shoulder to provide an elongate tubular body in which the ferrule 8200b can be located. The compression sleeve 8015b will now be described in more detail with reference to FIGS. 26a-26c.

FIG. 26A shows an end elevation of the compression sleeve 8015b. As shown in FIG. 26A, the compression sleeve has a substantially circular cross-section.

FIG. 26B shows a side elevation of the compression sleeve 8015b. As shown in this figure, the compression sleeve 8015b comprises a storage portion 8264 (the proximal end of which provides the proximally facing shoulder 8262 is formed) and a compression portion 8266 that extends from the storage portion towards an open proximal end. As can be seen in FIG. 26B, the compression portion 8266 has an outer diameter that tapers from a maximum diameter at the distal end to a minimum diameter at the proximal end.

FIG. 26C shows a cross-sectional side elevation through the plane F:F′ shown in FIG. 26A. As shown in FIG. 26C the compression portion 8266 has a constant internal diameter d_{c}. Because the compression portion 8266 has an outer diameter which tapers from a first outer diameter d_{d} at a distal end thereof (an end closest to the storage part 8264) to a second, smaller outer diameter dₚ at a proximal end thereof (an end furthest from the storage part 8264), the thickness of the wall 8268 of the compression part 8266 is tapered along its length, from a maximum thickness at the distal end of the compression sleeve part, to a minimum thickness at the proximal end of the compression sleeve part.

Owing to the variance in sleeve wall thickness, a hoop stress between the damper and the first drive component varies as a function of position of the first drive component relative to the damper. The variation in hoop stress creates a variation in the normal force between the first drive component and the damper. This results in a variation in the frictional engagement between the sleeve and the damper and hence a variation in the attenuation of the drive spring force as applied to the plunger and/or medicament container.

Although embodiments have been described with a damper positioned inside a first drive component, the present disclosure is not limited to these arrangements. For example, damping of the drive spring can be achieved using a damper, for example an annular damper, surrounding the first drive component so as to generate the friction force for attenuating the force of the drive spring in various stages of the advancement of the first drive component.

FIG. 27 shows a cross section of an example injection device 2001 (discussed with reference to FIG. 12) in which an annular damper 2200 is disposed around and engages an outer surface of a first drive element. FIG. 27 shows the proximal end of the device 2001 described with reference to FIG. 12.

An annular damper 2200 shown in FIG. 27 takes the form of an O-ring and is seated in a channel formed in the proximal housing 2032 so as to be fixedly coupled to the housing in the longitudinal direction of the housing. The annular damper 2200 surrounds and engages an outer wall of a first drive component, which here takes the form of latch 2040. In its relaxed state, the annular damper 2200 has an inner diameter which is slightly less than the outer diameter of the outer wall of the first drive component (latch 2040). The first drive component (latch 2040) is connected to the drive spring 2017 by a drive sleeve that takes the form of a latch extension 2042. Once surrounding the first drive component (latch 2040) the annular damper 2200 therefore exerts a normal force on the outer surface of the first drive component (latch 2040) and the resulting friction force between the annular damper 2200 and the first drive component (latch 2040) acts to oppose the force generated by the drive spring 2017.

In FIG. 27, the latch 2040 is arranged to be in contact with the annular damper 2200 throughout the driving of the injection device has a substantially uniform outer diameter and therefore the attenuation force is independent of the extension position of the first drive component. However, embodiments are not limited thereto. For example, the latch 2040 may have a varying outer diameter so that at various stages of the extension of the drive spring 2017, different magnitudes of normal force are exerted by the annular damper 2200 on the first drive component 2015′. Alternatively, or in addition, the outer diameter of the latch 2040 may vary such that in some stages of the extension of the drive spring, there is contact between the annular damper 2200 and the latch 2040 and in other stages there is no contact. Thus, by varying the outer diameter of the sleeve, various friction force profiles can be achieved including, but not limited to, any of those described herein.

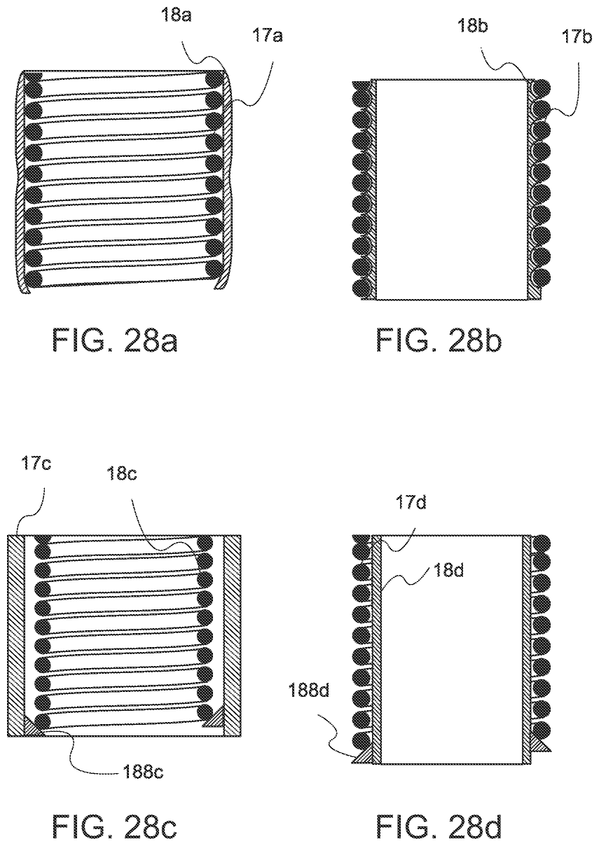

Although embodiments describe a fixed damper which exerts a friction force on a component driven by the drive spring, the inventors have recognised that other means of attenuating the force of the drive spring are also possible. For example, FIGS. 28a-28d show different configurations of an elastomer in direct contact with a drive spring in its compressed state. The elastomer is arranged to resist uncoiling of the drive spring, or to allow the individual coils to advance only one at a time.

FIG. 28A shows an elastomer in the form of an elastomer sheath 18a surrounding the drive spring 17a in its fully compressed state. The elastomer sheath 18a in its relaxed state has an inner diameter which is less than the outer diameter of the drive spring 17a. The elastomer sheath 18a is longer than the drive spring 17a in its fully compressed state. Therefore, the elastomer sheath 18a effectively shrink wraps the drive spring 17a when it is stretched to surround the drive spring 17a in that the ends of the elastomer sheath extending past either end of the drive spring 17a shrink so as to exert a compressive force on the drive spring. Therefore, an axial force is exerted on the coil at each the end of the drive spring 17a (i.e. the force is exerted in a direction parallel to the axis of the drive spring 17a). The force exerted by the elastomer sheath 18a prevents the coils from extending all at once. Only one coil at a time may be released from the end of the elastomer sheath 18a. Once one coil is released, the axial force of the elastomer sheath 18a is exerted on the next coil so as to resist its extension and so on.

FIG. 28B shows an elastomer inner tube 18b which works in substantially the same way as the elastomer sheath 18a of FIG. 28A. The elastomer inner tube 18b is also positioned concentrically with the drive spring, except that it is formed on the inside of the drive spring 17b. The elastomer inner tube 18b has an outer diameter which is greater than the inner diameter of the drive spring 17b and the elastomer inner tube 18b is longer than the compressed drive spring 17b so that the ends of the elastomer inner tube 18b exert an axial force on the ends of the drive spring 17b to achieve the same effect as described above with reference to FIG. 28A.

FIG. 28C shows a rigid support tube 18c arranged concentrically with and surrounding the drive spring 17c. The support tube 18c supports an elastomer ridge 188c arranged circumferentially on the inside surface of one end of the support tube 18c. The elastomer ridge 188c has an inner diameter which is smaller than the outer diameter of the drive spring 17c so that it exerts an axial force on the end of the drive spring 17c to resist the uncoiling of the first coil. Once the first coil is released from the end of the support tube 18, the elastomer ridge 188c exerts an axial force on the next coil and so forth.

FIG. 28D shows a similar arrangement to FIG. 28C except that it includes an inner support tube 18d which is arranged inside the drive spring 17d and outer elastomer ridge 188d is arranged circumferentially on the outer surface at the end of the support tube 18d. The outer elastomer ridge 188d has an outer diameter which is larger than the inner diameter of the drive spring 17d so as to exert an axial force on the end of each individual coil before they are released sequentially from the end of the support tube. Thus, the aforementioned effect of releasing the coils one by one is also achieved with this apparatus.

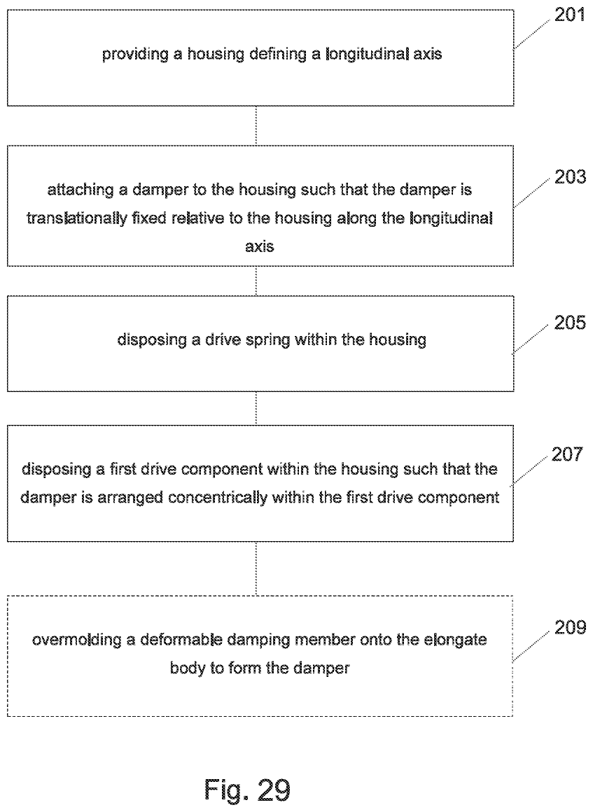

FIG. 29 shows a method of manufacturing an injection device according to the disclosure. At step 201, a housing is provided defining a longitudinal axis. At step 203, a damper is attached to the housing such that the damper is translationally fixed relative to the housing along the longitudinal axis. At step 205, a drive spring is disposed within the housing, and at step 207, a first drive component is disposed within the housing such that the damper is arranged concentrically within the first drive component, wherein the first drive component is configured to be driven by the drive spring along the longitudinal axis relative to the damper such that the damper and the first drive component frictionally engage. An additional step, step 209, can include overmolding a deformable damping member onto an elongate body (e.g. a mandrel) to form the damper.

As the reader will appreciate, the steps above can be carried out in any order. The method may further comprise providing any of the features described above with reference to the embodiments shown in FIGS. 15-28d.

In accordance with the description of the apparatus in this disclosure, there is also provided a method of damping a drive spring in an injection device, the method comprising the steps of:
advancing, using a drive spring, a medicament container from retracted position to an extended position relative to a housing of the injection device, wherein the drive spring delivers a drive force to the medicament container via a first drive component;
moving the first drive component relative to a damper, the damper being concentrically arranged with respect to the first drive component;
providing frictionally engagement between a surface of the first drive component and the damper during movement of the drive component relative to the damper.

As may be understood by the foregoing description of embodiments, the frictional force between the damper and first drive component may be adjusted by affecting the normal force between and/or the kinetic frictional coefficient between these components. The normal force may be affected by the fit between the damper and first drive component and/or the resilience or resistance to deformation of either component, and/or the displacement of a part of either component (the displacement usually being proportional to a restorative force in the case of an elastomer). The kinetic frictional coefficient is affected by the contact area and/or the kinetic frictional coefficient per unit contact area between the damper and the first drive component.

As the skilled person will understand from the described embodiments, the damper or damping member(s) may be of uniform cross section. However, this disclosure is not limited thereto. For example, the damper may have a variable cross section so as to vary the normal force between the damper and first drive component depending on the position of the damper relative to a compression zone of the first drive component. This is yet another means by which the attenuation of the drive spring force may be varied depending on the relative position of the damper and first drive component.

The preceding detailed description describes systems and methods for force damping in an injection device having a specific needle driving and retraction mechanism. However, the skilled person will understand that the invention is not limited to use in connection with the example injection device described here. Rather, one or more benefits associated with the present invention may be implemented in connection with other drug delivery devices, as will be apparent to the skilled person in light of the preceding detailed description.

Although a drive spring is described, the inventors have recognised that embodiments may be more generally described as having an elastic member (of which a drive spring is just one example).

Although a damper is described, the injection may include a damping system including a multiple damping components as well as two or more components which are capable of damping the drive spring force when interacting with one another. Moreover, although an injection device is described, embodiments may be said to relate to a damping system for an injection device or a damping system for a drive assembly of an injection device.

Damping arrangements according to the disclosure may be implemented in an injection device configured to automatically discharge a dose of medicament. More particularly, damping arrangements according to the disclosure may be employed in autoinjectors of the type that advance a medicament container, discharge a dose of medicament, and automatically retract the medicament container relative to the housing after use.

The damping arrangements described herein may be combined with a power pack according to the aspect described above and/or one or more of a connection assembly, and a passive safety shield, each of which is described in more detail below.

### Connection Assembly

The disclosure also provides an example connection assembly configured to connect a needle for delivering an injection with the interior volume of a sealed medicament container.

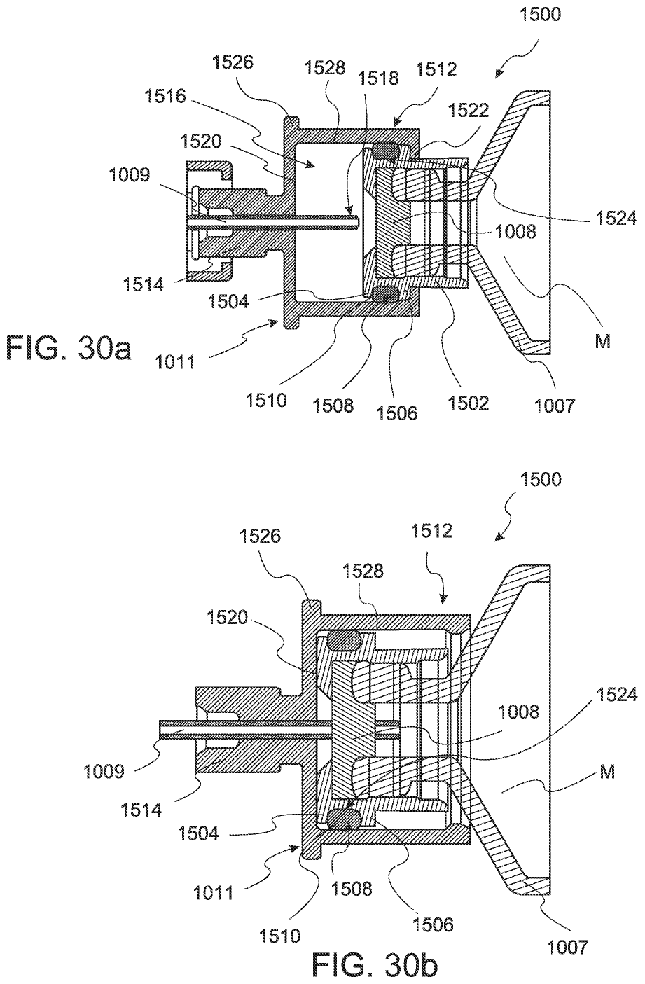

FIG. 30A shows a cross-sectional view of an assembly 1500 for an injection device such as that shown in FIG. 1 for injecting a medicament according to the present disclosure. In FIG. 30A, the assembly is shown in a storage state. The assembly 1500 includes a medicament container 1007, containing medicament M, having a container cap 1502. The container 1007 is sealed by a septum 1008. The cap 1502 has a first rib 1504 and a second rib 1506. The first and second ribs 1504 and 1506 extend around the circumference of the cap 1502 and define a first positioning recess 1508 therebetween. A sealing element 1510 is in contact with an external surface 1524 of the cap 1502 and is disposed between the first and second ribs 1504 and 1506 in the first positioning recess 1508. The sealing element 1510 extends around the circumference of the cap 1502. The sealing element 1510 is chemically bonded to, specifically over moulded onto, the cap 1502 such that the cap and sealing element form a single body. The sealing element is made of a flexible material and is compressed between the two ribs 1504 and 1506 (as well as being chemically bonded to the cap 1502).

A distal portion of the cap 1502, including the first and second ribs 1504 and 1506, and the sealing element 1510 sit within the needle hub 1011. The needle hub 1011 comprises a main body 1512 and an elongate portion 1514 extending from the main body 1512. Within the main body 1512 of the needle hub 1011, surfaces of the needle hub 1011, the cap 1502 and the sealing element 1510 define a cavity 1516 within which a free end 1518 of the needle 1009 sits. Referring to the internal surfaces of the needle hub 1011, the needle hub comprises a first inner surface 1520 which is circular and extends perpendicular to the needle 1009, facing the cap 1502. The needle hub 1011 also comprises a proximal protrusion 1522 which extends radially inwards, towards the needle 1009. The proximal protrusion 1522 is annular and extends around and can be in contact with the external surface of the cap 1502. The proximal protrusion 1522 is also selectively in contact with the second rib 1506, specifically a proximal face of the second rib. In this way, the needle hub 1011 encloses a distal end of the cap 1502, including the first and second ribs 1504 and 1506 and the sealing element 1510.

The needle hub 1011 also comprises a second inner surface 1528, which is tubular, and extends parallel to the needle 1009, connecting the first inner surface 1520 and the proximal protrusion 1522. The needle hub 1011 is made of a rigid material and the flexible sealing element 1510 is compressed between the first and second ribs 1504 and 1506 of the cap 1502 and also against the second inner surface 1528 of the needle hub 1011. Accordingly, a seal is formed between the external surface 1524 of the cap 1502 and the second inner surface 1528 of the needle hub 1011. The seal is provided by the sealing element 1510.

The needle 1009 extends through the first inner surface 1520 of the needle hub and through elongate portion 1514 of the needle hub. The needle 1009 in turn is, at a distal end of the assembly 1500, in contact with a needle shield (corresponding to the needle shield 1004 shown in FIG. 1).

At a distal end of the main body 1512 of the needle hub 1011, the needle hub comprises an annular protrusion 1526. The purpose of this protrusion will be described below with reference to FIG. 30B.

FIG. 30A shows the assembly 1500 in a first (pre-injection) state, in which a free end 1518 of the needle 1009 is held away from the septum 1008. FIG. 30B shows the needle hub assembly in a second state in which the needle 1009 has pierced the septum 1008. The process by which the assembly 1500 transitions from the state shown in FIG. 30A to that shown in FIG. 30B will now be described.

The process of carrying out an injection is described above with reference to FIG. 1, and as the reader will understand applies equally to this embodiment. As described, during an injection, the needle hub 1011 and medicament container 1007 advance forwards in a distal direction so that the hypodermic needle 1009 pierces the injection site. Continued advancement of the plunger rod 1015 (see FIG. 1) then further advances the medicament container 1007. As the medicament container 1007 advances forward, it moves relative to the needle hub 1011 and as the container 1007 moves, the seal between the needle hub 1011, specifically the second inner surface 1528 of the needle hub 1011, and the external surface 1524 of the cap 1502 is maintained. The needle 1009 then pierces the septum 1008 to allow the medicament M to move from the medicament container 1007 and through the hypodermic needle 1009 to be dispensed. Plunger 1013 is moved through the medicament container 1007 and towards the septum 1008 thereby expelling medicament M from the medicament container 1007 through the hypodermic needle 1009. An injection is thereby performed.

As the needle hub 1011 advances in a distal direction during the injection, annular protrusion 1526 on the needle hub 1011 engages the flexible latch arms 1402 (see FIGS. 47a-c and the associated description below) and pushes them radially outwards. This disengages the flexible latch arms 1402 from the latching surfaces 1404. This has the effect that the safety shield 1019 is no longer locked in the retracted position.

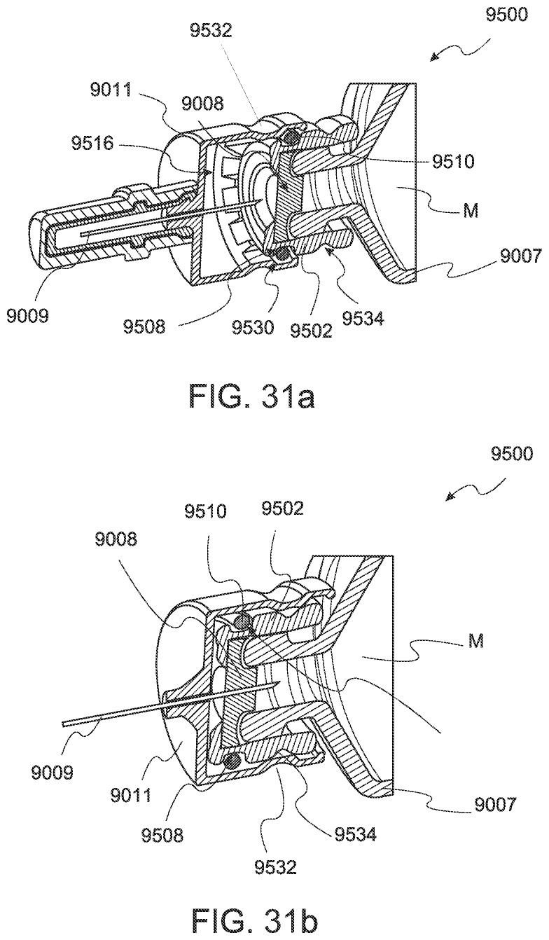

Another assembly for use in an injection device for injecting a medicament will now be described with reference to FIGS. 31a and 31b. The assembly shown in FIG. 31A is similar to that shown in FIGS. 30a and 30b with the following differences. Firstly, the sealing element 9510 is an O-ring. As before, the sealing element 9510 is made of a flexible material. The sealing element 9510 is disposed within a positioning recess 9508 on the cap 9502, which is made of a rigid material. The needle hub 9011, also made of a rigid material, has a corresponding recess 9530. When the assembly 9500 is in the first state, in which a free end of the needle 9009 is held away from the septum 9008, the recess 9530 on the needle hub 9011 aligns with the sealing element 9510 and aids in compressing the sealing element 9510 into the positioning recess 9508 on the cap 9502. A seal is formed by the sealing element 9510 between the cap 9502 and the needle hub 9011, to form cavity 9516. During an injection, as the container 9007 is advanced forward (in a distal direction) relative to the needle hub 9011, as described above, the seal between the needle hub 9011 and the cap 9502 is maintained.

FIG. 31B shows the assembly 9500 of FIG. 31A in the second state, i.e. when the needle 9009 has pierced the septum 9008. The sealing element 9510 is compressed in the first positioning recess 9508 of the cap 9502 against an inner surface of the needle hub 9011. The positioning protrusion 9532 on the needle hub 9011 aligns with and locks into a second positioning recess 9534 on the cap 9502. This second positioning recess 9534 is adjacent the positioning recess 9530. The interlocking of the second positioning recess 9534 with the positioning protrusion 9532 on the needle hub prevents the needle hub moving in a proximal direction relative to cap 9502 (thus removing the needle 9009 from the container 9007) once the assembly 9500 is in the second state.

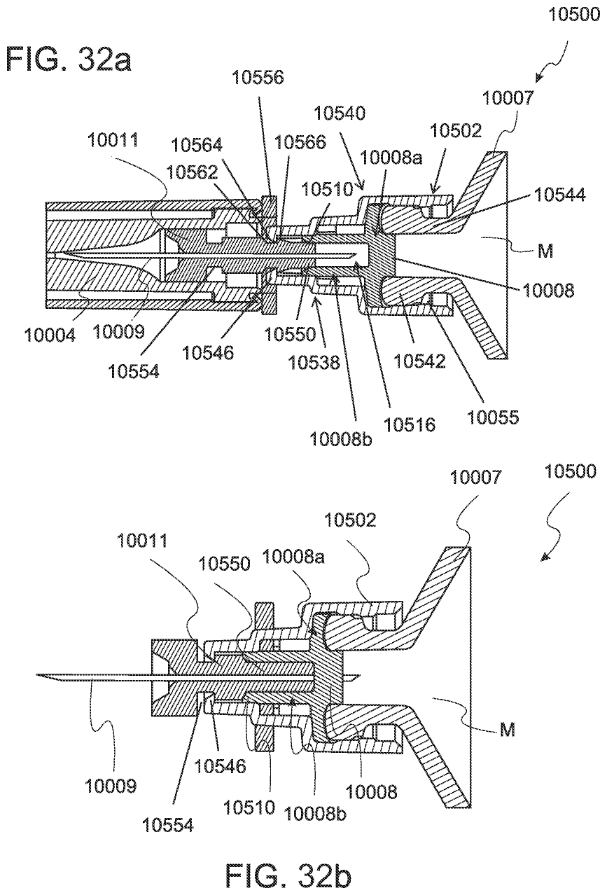

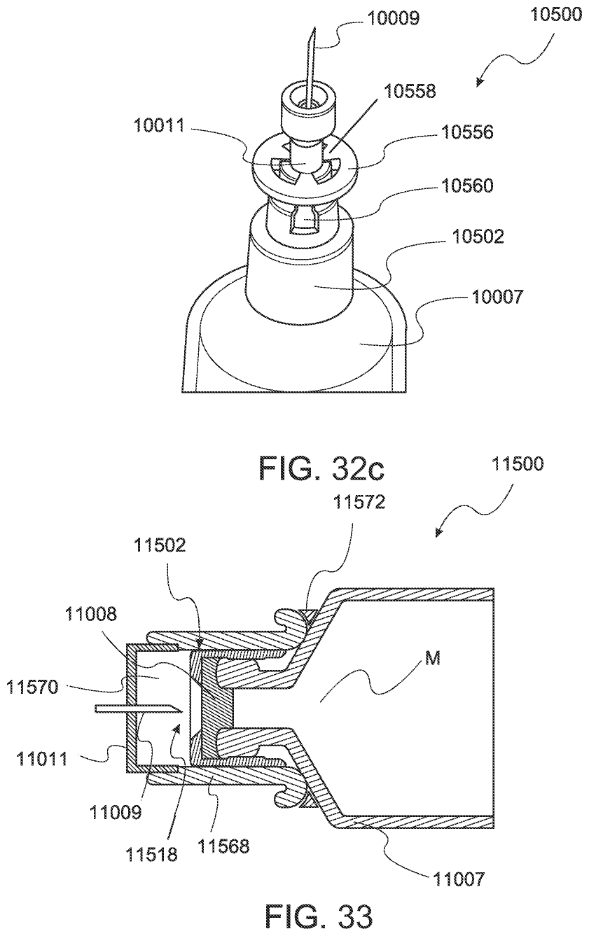

A third assembly 10500 is shown in FIG. 32A. This third assembly has elements in common with the previous two assemblies (shown in FIGS. 30a, 30b, 31a and 31b) but has the following differences. Firstly, the assembly 10500 is configured so that the needle hub is disposed inside of the cap 10502 (as opposed to the assemblies shown in FIGS. 30a, 30b, 31a and 31b, in which the cap was disposed inside of the needle hub). When the assembly 10500 is in the first (pre-injection) state, part of the needle hub 10011 is disposed inside of the cap 10502.

The septum 10008 also has a different shape and additional functionality as compared with the septum in FIGS. 30a, 30b, 31a and 31b. The septum 10008 has a main portion 10008a and an elongate portion 10008b which defines a cavity 10516. The elongate portion 10008b is an annular ridge. The needle hub 10011 also has an elongate portion 10550.

The sealing element 10510 is provided by part of the septum 10008, specifically a distal end of the elongate portion 10008b, and is disposed between an internal surface of the cap 10502 and an external surface of the needle hub 10011. The cap 10502 and the needle hub 10011 are both made of a rigid material (which may or may not be the same material) and the septum is made of a flexible material. The annular ridge of the septum is thus compressed between the cap 10502 and the needle hub 10011 and a seal is formed, thereby forming a channel or cavity 10516.

The cap 10502 comprises a first shoulder 10538 and a second shoulder 10540. At each shoulder (moving from a distal end to a proximal end of the cap) the radius of the cap abruptly increases. The sealing element (i.e. a distal end of the septum) is compressed between the first shoulder 10538 of the cap and the needle hub 10011. The sealing element is therefore compressed against a well-defined point of the cap, ensuring a tight seal. The portion of the cap which is proximal to the first shoulder 10538 has a larger radius than the distal end of the cap so as not to unduly restrict movement of the container and septum relative to the needle hub 10011. The second shoulder 10540 of the cap provides a compressive seal on the septum.

The cap 10502 surrounds a proximal end of the container 10007. The proximal end of the container 10007 has a first cylindrical portion 10542 with a first radius and a second cylindrical portion 10544 with a second radius which is smaller than the first radius. The second portion 10544 is further from the septum 10008 than the first portion 10542. The cap 10502 is in contact with the container 10007 over the first portion and comprises a rib 10055 which interlocks with the second portion (which has a reduced radius as compared to the first portion). This aids in securing the cap to the container 10007.

At the distal end of the cap 10502 is an opening which receives a portion of the needle hub 10011. At the opening, the cap 10502 comprises a protrusion 10546 which projects radially inward. The needle hub comprises a protrusion 10566 which interlocks with the protrusion 10546 of the cap and prevents the needle hub 10011 from moving in a distal direction relative to the cap 10502 and hence disengaging from the cap. The protrusion 10546 of the cap is sloped such that the protrusion at a distal end has a thickness which is smaller than that of the protrusion at a proximal end. The needle hub 10011 has a sloped face 10562 which engages with the sloped face of the protrusion 10546 on the cap 10502 as the assembly transitions from the first state to the second state, as will be described below. The needle hub 10011 also comprises a recess 10554. This recess 10554 interlocks with the protrusion 10546 of the cap when the device is in the second state and prevents the needle hub from moving in a distal direction relative to the cap once the assembly has reached the second state.

The needle hub 10011 also comprises a disc 10556 which extends radially outwards from the needle hub 10011. The disc 10556 comprises three apertures, evenly spaced circumferentially around the needle hub 10011. These can be seen in FIG. 32C. The apertures define three wings 10558, which separate the apertures. The apertures receive a distal end of the cap of the container when the device is in the first state and prevent the needle hub from twisting relative to the cap as the device transitions from the first state to the second state. This will be described in greater detail below.

The cap comprises three corresponding recesses 10560 configured to receive the three wings 10558 of the needle hub 10011 as the needle hub moves in a proximal direction relative to the cap.

During transition from the first state to the second state, the container 10007 moves distally, relative to the needle hub 10011, and as this happens, the elongate portion 10550 of the needle hub 10011 is received inside channel or cavity 10516 of the septum 10008. The elongate portion 10550 of the needle hub 10011 provides a continuous surface against which the sealing element 10510 is compressed by the cap 10502. In this way, the sealing element 10510 is continuously compressed between an internal surface of the cap 10502 and an external surface of the needle hub 10011 during transition of the assembly 10500 from the first state (shown in FIG. 32A) to the second state (shown in FIG. 32B). This smooth surface of the needle hub 10011 means that the needle hub is easier to manufacture. A lack of positioning features also means that the sealing element is less likely to be damaged as the sealing element moves over the needle hub 10011.

As the needle hub 10011 moves, the sloped face 10562 of the needle hub moves over the sloped face 10564 of the protrusion 10546 on the cap and a distal end of the cap is pushed open by the width of the needle hub. The needle hub 10011 continues to move in a proximal direction relative to the cap and eventually, the needle 10009 pierces the septum 10008. A fluid path between the container and the needle is opened and the medicament M is dispensed. The protrusion 10546 on the cap interlocks with the recess 10554 of the needle hub (shown in FIG. 32B) and thus the needle hub is prevented from moving in a distal direction relative to the cap once it is in this position.

FIG. 32C shows an alternate view of the assembly 10500 when the device is in the first (pre-injection) state. As shown, a distal end of the cap 10502 is received in the apertures of the disc 10556 of the needle hub 10011. The cap comprises recesses 10560 to receive the wings 10558 of the disc 10556 as the needle hub moves in a proximal direction relative to the cap.

FIG. 33 shows a fourth assembly 11500. The assembly 11500 comprises a sealing sleeve 11568 surrounding the cap 11502. The sealing sleeve is in contact, at a proximal end, with an external surface of the container 11007 and is held in place by a retaining ring 11572, which surrounds the container 11007. The assembly 11500 also comprises a rigid needle hub 11011 through which the needle 11009 passes. The needle hub 11011 is in contact with an internal surface of the sealing sleeve 11568 at a distal end of the sealing sleeve. A seal is formed between the cap 11502 and the sealing sleeve 11568 and also between the needle hub 11011 and the sealing sleeve 11568. A cavity 11570 is defined by the needle hub 11011, the sealing sleeve 11568, the cap 11502 and the septum 11008. A free end 11518 of the needle 11009 sits within this cavity 11570 when the assembly is in a first (storage) state.

As a force is applied to the medicament container 11007 in a distal direction, the container 11007 moves distally, relative to the needle 11009 and needle hub 11011. As the medicament container advances, the sealing sleeve buckles, bending radially outwards, and the needle 11009 pierces the septum 11008. Medicament M thus moves through the needle 11009 and is dispensed.

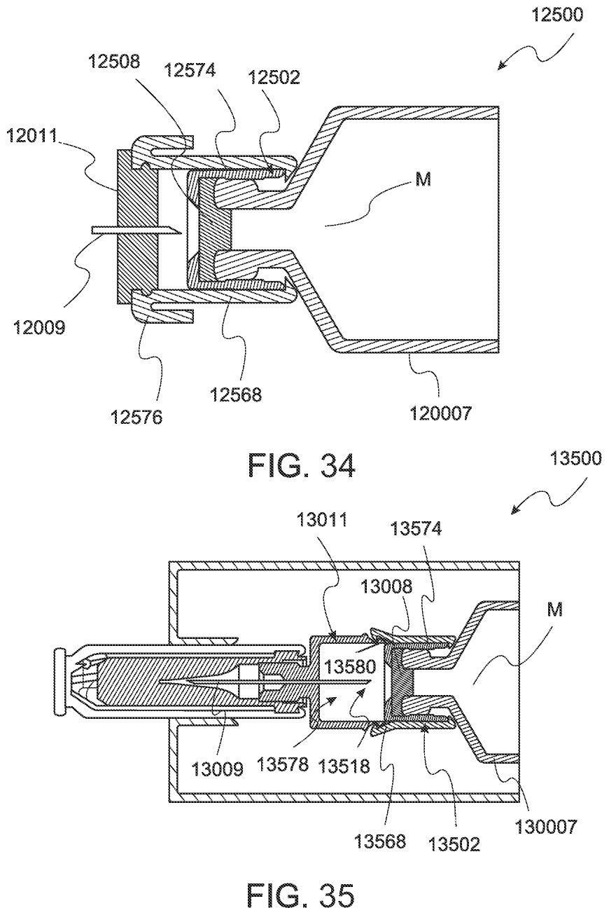

FIG. 34 shows a fifth assembly 12500. Like the assembly shown in FIG. 33, this fifth embodiment also employs a sealing sleeve 12568. The sealing sleeve 12568 surrounds the cap 12502 and extends radially inwards at a proximal end of the cap 12502, thus securing the sealing sleeve 12568 to the cap 12502. A seal is formed between an internal surface of the sealing sleeve 12568 and an external surface 12574 of the cap 12502.

At a distal end of the sealing sleeve 12568, the sleeve 12568 interlocks with a rigid needle hub 12011, through which the needle 12009 passes. A seal is formed between the needle hub 12011 and a distal end of the sealing sleeve 12568.

At a distal end of the sealing sleeve 12568, the sealing sleeve comprises a lip 12576 which extends around the sealing sleeve 12568. When the device is in the first state, the lip 12576 extends in a proximal direction.

When a force is applied to the medicament container in a distal direction, the container 12007 moves relative to the needle hub 12011 and the needle 12009. As the container 12007 advances, the sleeve 12568 buckles outwards (specifically the portion of the sealing sleeve 12568 that is not in contact with the external surface of the cap 12502 buckles outwards) and the lip 12576 inverts and instead extends in a distal direction, thus surrounding a distal end of the needle hub 12011. This helps to guide the needle hub and ensure it is located centrally relative to the container 12007. Eventually, the needle 12009 pierces the septum 12508 and the medicament M enters the needle 12009 and is dispensed.

FIG. 35 shows a sixth assembly 13500. This sixth assembly 13500 also employs a sealing sleeve 13568. In this configuration, the sealing sleeve 13568 surrounds the cap 13502 and extends radially inwards at a proximal end of the cap 13502, thus securing the sealing sleeve 13568 to the cap 13502. At a distal end of the sealing sleeve 13568 there is a ridge which interlocks with a corresponding ridge on a proximal end of the needle hub 13011, thus forming a seal between the sealing sleeve 13568 and the needle hub 13011. The needle hub 13011 is made of a rigid material and the sealing sleeve 13568 is made of a flexible material.

A seal is also formed between an internal surface of the sleeve 13568 and an external surface 13574 of the cap 13502. A cavity 13578 is defined by the internal walls of needle hub 13011 and cap 13502 and the septum 13008. A free end 13518 of the needle 13009 sits in this cavity 13578 when the assembly is in a first state.

When a force is applied to the medicament container 13007 in a distal direction, the container 13007 moves distally relative to the needle 13009 and needle hub 13011. As the container advances, the wall 13580 of the needle hub 13011 moves underneath (specifically, radially inwards of) the sealing sleeve 13568. As this happens, a seal is maintained between the sealing sleeve 13568 and an external surface of the needle hub 13011. Eventually, the needle 13009 pierces the septum 13008 and the medicament M enters the needle 13009 and is dispensed.

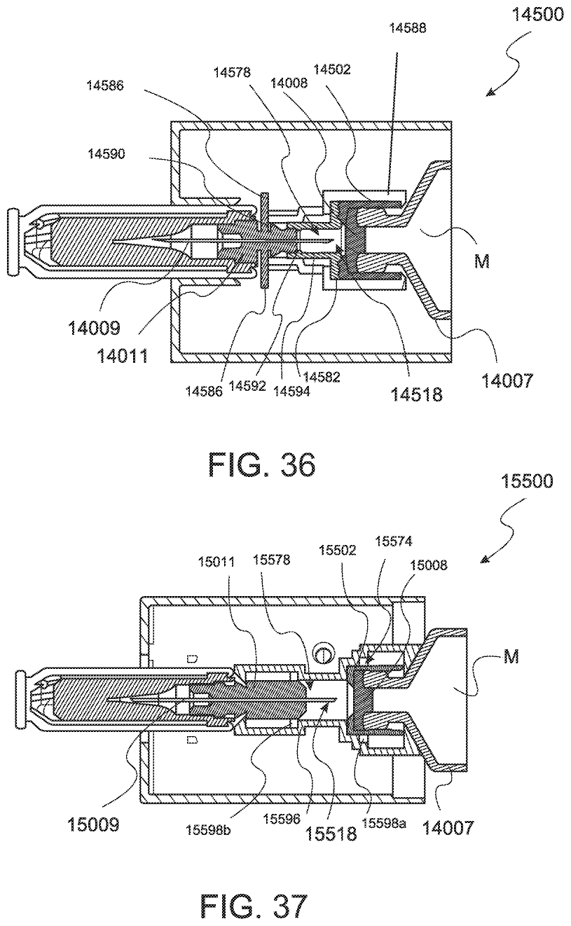

FIG. 36 shows a seventh assembly 14500. The assembly 14500 in FIG. 36 comprises a sealing element in the form of a stopper element 14582 in contact with a cap 14502 of the container 14007. Alternatively, the stopper element 14582 may be in contact with a first outer cap (not shown) which surrounds the cap 14502 of the container 14007. The benefit of using an outer cap in this way is that the container 14007 and its cap 14502 do not need any modification or to have a particular shape or any particular features to be used with the assembly 14500. The stopper element is made of a flexible material.

In contact with the stopper element is the needle hub 14011, which sits partially inside (i.e. radially inwards of) the stopper element 14582 when the assembly is in a first state. The needle hub 14011 is made of a rigid material and has wings 14586 for stability. A second outer cap 14588 surrounds the cap 14502, the stopper element 14582 and part of the needle hub 14011. This outer cap 14588 comprises a series of channels which the wings 14586 move into as the assembly transitions from the first state to the second state. This interlocking of the wings 14586 with the channels of the second outer cap 14588 prevents the needle hub from rotating relative to the stopper element 14582 as the assembly transitions to the second state. The channels of the outer cap 14588 are not shown in FIG. 36 but are similar to those of the cap 10502 shown in FIG. 32C. Further, the wings 14586 prevent the needle hub 14011 from moving too far in a proximal direction, as will be described below.

Adjacent to the wings 14586 of the needle hub is a recess 14590 which interlocks with a corresponding ridge 14592 at a distal end of the stopper element 14582 when the device is in the second state.

A seal is formed between an external surface of the needle hub 14011 and an internal surface of the stopper element 14582 in this first state (shown in FIG. 36). A cavity 14578 is defined by the stopper element 14582, the needle hub 14011, a distal end of the cap 14502 and the septum 14008. A free end 14518 of the needle 14009 sits inside this cavity 14578 when the assembly 14500 is in the first state.

When a force is applied to the medicament container 14007 in a distal direction, the container 14007 (along with the second outer cap 14588 and stopper element 14582) moves distally relative to the needle 14009 and needle hub 14011. As the container advances, the proximal end of the needle hub 14011 moves underneath (specifically, radially inwards of) a wall 14594 of the stopper element 14582. As this happens, a seal is maintained between the stopper element 14582 and an external surface of the needle hub 14011. The needle hub 14011 moves relative to the stopper element 14582 and eventually, the needle 14009 pierces the septum 14008 and the medicament M enters the needle 14009 and is dispensed. The wings 14586 of the needle hub 14011, having slotted into channels in the second outer cap 14588 as discussed above, eventually abut the distal end of the stopper element 14582. The needle hub 14011 is thus prevented from moving any further in a proximal direction, relative to the stopper element 14582. The recess 14590 of the needle hub 14011 also interlocks with the corresponding ridge 14592 on the stopper element 14582, thus preventing the needle hub 14011 from moving back in a distal direction relative to the stopper element 14582.

FIG. 37 shows an eighth assembly. The assembly 15500 of this configuration comprises an assembly container 15596 that contains and surrounds a proximal end of the needle 15009, a proximal end of the needle hub 15011, and a first sealing element and a second sealing element in the form of two flexible rings 15598a and 15598b. The rings 15598a and 15598b are made of a flexible material whereas the container 15596 is made of a rigid material. The first ring 15598a is disposed between an internal wall of the container 15596 and an external surface 15574 of the cap 15502. A seal between the internal surface of the container 15596 and an external surface 15574 of the cap 15502 is provided by the first ring 15598a. The second ring 15598b is disposed between an internal wall of the container 15596 and an external surface of the needle hub 15011. A seal between the internal surface of the container 15596 and an external surface of the needle hub 15011 is provided by the second ring 15598b. A cavity 15578 is defined by internal walls of the container 15596, a proximal end of the needle hub 15011 and a distal end of the cap 15502 and is sealed by rings 15598a and 15598b. A free end 15518 of the needle 15009 sits within this cavity 15578 when the assembly 15500 is in a first state (shown in FIG. 37).

When a force is applied to the medicament container 15007 in a distal direction, the container 15007 moves distally relative to the needle 15009 and needle hub 15011. As the container 15007 advances, the distal end of the container 15596 flexes open and moves radially outwards of the distal end of the assembly. Also as the container advances, a seal is maintained between the needle hub 15011 and the assembly container 15596 (by the second ring 15598b) and between the cap 15502 and the assembly container 15596 (by the first ring 15598a). Eventually, the needle 15009 pierces the septum 15008 and the medicament M enters the needle 15009 and is dispensed.

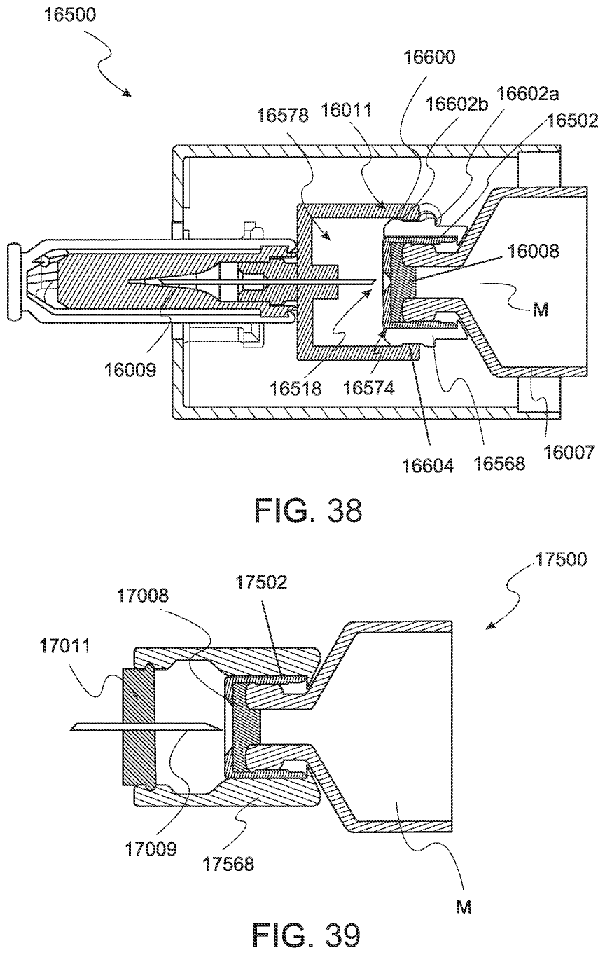

FIG. 38 shows a ninth assembly 16500. This ninth assembly, like that shown in FIG. 35, for example, also employs a sealing sleeve 16568. The sealing sleeve 16568 is made of a flexible material. In this embodiment, the sealing sleeve 16568 surrounds the cap 16502 and extends radially inwards at a proximal end of the cap 16502, thus securing the sealing sleeve 16568 to the cap 16502. At a distal end of the sealing sleeve 16568, the sealing sleeve 16568 is disposed between an internal surface 16600 of the needle hub 16011 and an external surface 16574 of the cap 16502. A seal between the cap 16502 and the internal surface 16600 of the needle hub 16011 is provided by the sealing sleeve 16568. A cavity 16578 is defined by the internal walls of needle hub 16011, the cap 16502 and the septum 16008 and the cavity 16578 is sealed by the sealing sleeve 16568. A free end 16518 of the needle 16009 sits in this cavity 16578 when the assembly 16500 is in a first state.

The external surface of the sealing sleeve 16568 comprises positioning features to retain the proximal end of the needle hub 16011 when the assembly 16500 is in the first state. In this case, the positioning features are ridges 16602a and 16602b. A ridge 16604 on the proximal end of the needle hub 16011 is disposed between the ridges 16602a and 16602b when the assembly is in the first state and holds the needle hub 16011 relative to the container 16007.

When a force is applied to the medicament container 16007 in a distal direction, the container 16007 moves distally relative to the needle 16009 and needle hub 16011 and the ridge 16602b of the needle hub 16011 moves over the proximal ridge 16602a of the sealing sleeve 16568. As the container advances, a seal is maintained between the cap 16502 and an internal surface 16600 of the needle hub 16011. Eventually, the needle 16009 pierces the septum and the medicament M enters the needle 16009 and is dispensed.

FIG. 39 shows a tenth assembly 17500. This assembly is largely similar to that depicted in FIG. 34 except for the following differences. The assembly 17500 of FIG. 39 does not comprise the lip 12576 shown in FIG. 34 (however it will be appreciated that it could be incorporated here). As in the assembly of FIG. 34, the assembly of FIG. 39 comprises a sealing sleeve 17568. In the case of the assembly of FIG. 39, the region of the sealing sleeve 17568 which is not in contact with the external surface of the cap 17502 (i.e. the region of the sealing sleeve 17568 which is disposed between the needle hub 17011 and the cap 17502) has a reduced thickness, as compared to the thickness of the sealing sleeve over the portions where it is in contact with the cap 17502 and the needle hub 17011 respectively. This is to increase the likelihood of the sealing sleeve buckling at this location (i.e. where the thickness is reduced), thus facilitating better control over the components of the assembly 17500.

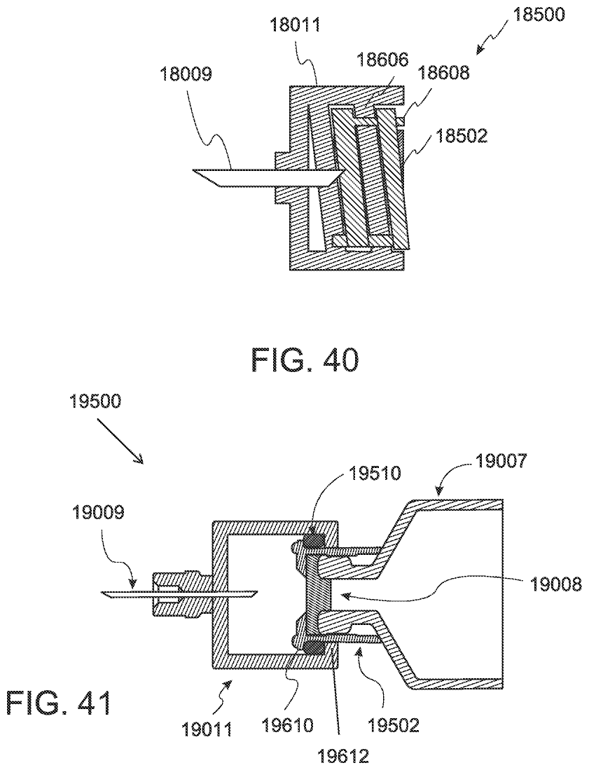

FIG. 40 shows an eleventh assembly 18500. In this assembly, the needle hub 18011 has internal threading 18606 of square cross-section. This threading is configured to interlock with corresponding threading provided by a sleeve 18608 which surrounds the cap 18502 of the medicament container. The sleeve 18608 is made of a flexible material. When the assembly 18500 is in the first state, i.e. before the needle 18009 has pierced the septum (not shown) of the cap 18502, the threading on the sleeve forms a seal with the threading on the internal surface of the needle hub 18011.

When a force is applied to the medicament container (not shown) in a distal direction, the threads on the sleeve 18608 and the threads on the needle hub 18011 skip over each other and the container moves distally relative to the needle 18009 and needle hub 18011. As the container advances, the needle 18009 pierces the septum and the medicament enters the needle 18009 and is dispensed.

FIG. 41 shows a twelfth assembly. This assembly 19500 is similar to that shown in FIG. 30A. In this case, however, the sealing element 19510 is bonded to the internal surface of the needle hub 19011, as opposed to the external surface of the cap 19502. Further, only a single rib 19610 is present on the external surface of the cap (which corresponds to rib 1504 in FIG. 30A). The sealing element 19510 is compressed between the rib 19610 and the proximal protrusion 19612 of the needle hub 19011. The assembly otherwise operates in the same way as described with reference to FIGS. 30a and 30b.

It will be appreciated that rib 19610 may not be present and that the sealing element may be prevented from moving in a proximal direction relative to the needle hub by friction between the sealing element and the cap.

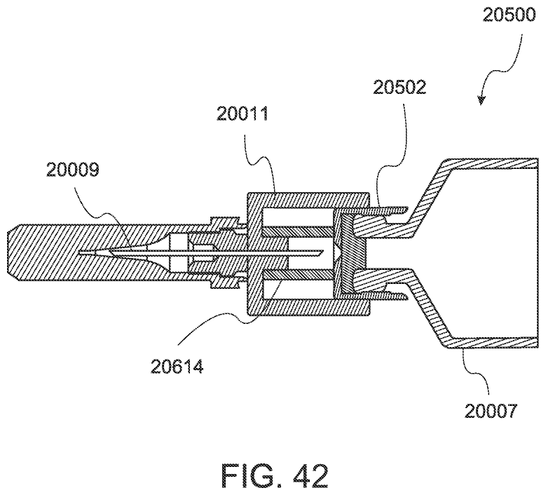

FIG. 42 shows a thirteenth assembly 20500. This assembly is similar to that shown in FIGS. 30a and 30b, except that instead of a sealing element surrounding the cap 20502 which forms a seal with an internal surface of the needle hub 20011, there is a sealing element in the form of a sleeve 20614 surrounding the needle 20009. The sleeve 20614 extends from the internal wall of the needle hub 20011 (which is perpendicular to the needle 20009 and is at the distal end of the needle hub 20011) to the distal end of the cap, facing the needle 20009. In this way, the sleeve 20614 surrounds the needle and sterility of both the needle and the area of the septum which is pierced by the needle is maintained at all times.

When a force is applied to the medicament container 20007 in a distal direction, the container 20007 moves distally relative to the needle 20009 and needle hub 20011. The sleeve 20614 buckles radially outwards as the container advances. Eventually, the needle 20009 pierces the septum and the medicament M enters the needle 20009 and is dispensed.

The sealing element in any of the embodiments described above may be made from any malleable elastomer, for example Santoprene. Specifically, Santroprene 101-73 could be used.

The cap and the needle hub in any of the embodiments described above may be made from a rigid polymer, such as polypropylene.

The needle in any of the embodiments described above may be made from a metal such as stainless steel (for example grade 304 or 316).

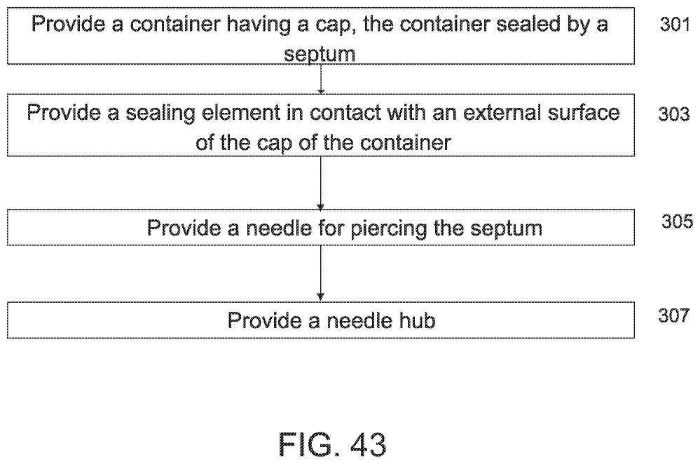

FIG. 43 shows a flow diagram demonstrating a method of manufacturing an assembly for an injection device for injecting a medicament. The method comprises the following steps.

At step 301, a container having a cap and which is sealed by a septum is provided. At step 303, a sealing element in contact with an external surface of the cap of the container is provided.

At step 305, a needle for piercing the septum is provided. The sealing element is disposed between the external surface of the cap of the container and an internal surface of the needle hub. At step 307, a needle hub is provided. The assembly is configured to transition from a first state, in which the needle is held away from the septum and a free end of the needle sits in a cavity sealed by the sealing element, to a second state, in which the needle passes through the septum. A seal between the internal surface of the needle hub and the external surface of the cap is provided by the sealing element when the assembly is in the first state. The seal is maintained throughout transition from the first state to the second state and is maintained when the assembly is in the second state.

Connection assemblies according to the disclosure may be implemented in an injection device configured to automatically discharge a dose of medicament. However, it will be understood that the connection assemblies described herein may be implemented in a manually actuated drug delivery device or a drug delivery device comprising motorised drive means. In at least some embodiments, the connection assemblies damping arrangements according to the disclosure may be employed in autoinjectors of the type that advance a medicament container, discharge a dose of medicament, and automatically retract the medicament container relative to the housing after use.

The connection assemblies described herein may be combined with a power pack and/or damping mechanism according to the aspects described above and/or a passive safety shield, which is described in more detail below.

### Passive Needle Injury Protection

The disclosure also provides an example safety device configured to protect a user from needle stick injuries before, during and after use of the device.

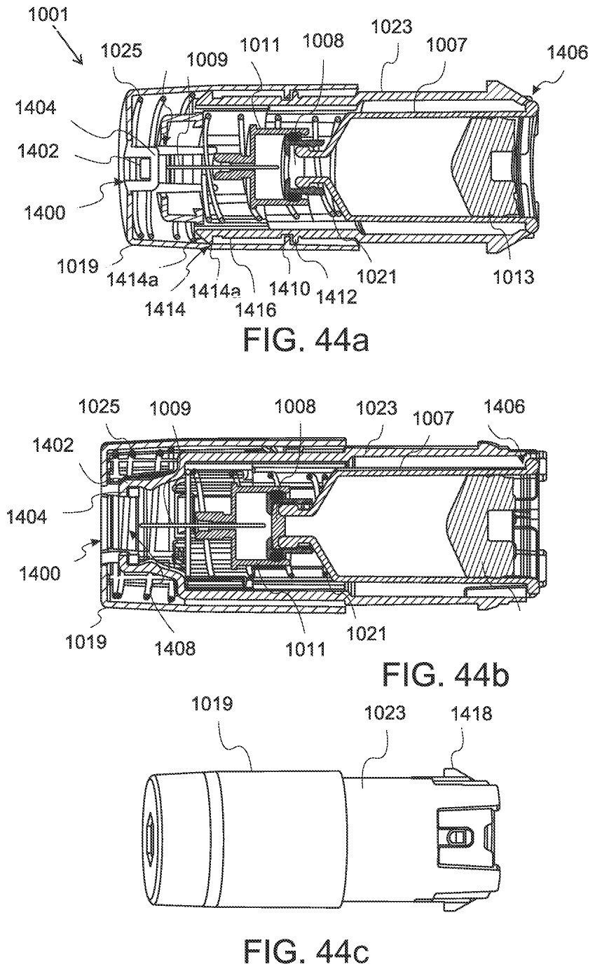

FIGS. 44a and 44b show cross-sectional views of the distal portion 1b of the injection device 1001, in the pre-injection configuration. The cross-section of FIG. 44B is perpendicular to the cross-section of FIG. 44A. FIG. 44C shows a side-view of the injection device of FIGS. 44A and 44B.

As shown in FIGS. 44a and 44b, the safety shield 1019 comprises an annular tube which is generally closed at its distal end, save for an opening 1400 through which the hypodermic needle 1009 extends during performance of an injection. The safety shield 1019 surrounds a distal portion of the housing 1023. FIGS. 44a-44c show the safety shield 1019 in a retracted position relative to the housing. Safety shield 1019 is advanceable in the distal direction relative to the housing 1023, into a deployed position for shielding the hypodermic needle 1009. Advancement spring 1025 biases the safety shied into the deployed position. Advancement spring 1025 is located between the housing 1023 and the safety shield 1019, such that a proximal end of the advancement spring 1025 acts on a distal-facing shoulder near the distal end of the housing 1023, and such that a distal end of the advancement spring 1025 acts on the distal end of the safety sleeve 1019. However, in the pre-injection configuration shown, flexible latch arm 1402 of the housing 1023 engages latching surface 1404 of the safety shield 1019 to prevent advancement of the safety shield 19. Accordingly, advancement of the safety shield 1019 into the deployed position under the influence of the advancement spring 1025, is prevented in the pre-injection configuration shown in FIGS. 44a-44c. Collectively, the flexible latch arms 1402 and the latching surfaces 1404 comprise a disengageable locking mechanism. The disengageable locking mechanism is in the locked configuration in FIGS. 44a-44c.

Housing 1023 includes an opening 1406 at its proximal end for receiving the medicament container 1007; and an opening 1408 at its distal end through which the hypodermic needle 1009, and a distal end of the needle hub 9, extend during performance of an injection. Medicament container 1007 is housed within the housing 1023, and is coupled with the needle hub 1011 and hypodermic needle 1009. In the pre-injection configuration of FIGS. 1a-1c, the medicament container 1007, needle hub 1011, and hypodermic needle 1009, are each in a first (retracted) position. Medicament container 1007, needle hub 1011, and hypodermic needle 1009, are configured to move distally through the housing 1023 from their first (retracted) position. Moreover, they are configured to move distally through the housing 1023 during performance of an injection, into a second (injection) position. The medicament container 1007, needle hub 1011, and hypodermic needle 1009, are biased into this first (retracted) position by the return spring 1021. The return spring 1021 is arranged between the housing 1023 and the medicament container 1007. A proximal end of the return spring 1021 acts against a distal-facing shoulder of the medicament container 1007. A distal end of the return spring 1021 acts against the distal end of the housing 1023. The medicament container 1007 is moveable into the second (injection) position under the influence of the plunger rod 1015 (not shown in FIGS. 44a-44c). That is to say, the plunger rod when activated will overcome the force of the return spring 1021 to advance the medicament container 1007 into the second (injection) position. Because the medicament container 1007 is coupled with the needle hub 1011, advancement of the medicament container 1007 towards the second (injection) position also causes advancement of the needle hub 1011 (and the hypodermic needle 1009) into the second (injection) position, such that the hypodermic needle 1009 pierces an injection site. Further advancement of the medicament container 1007 into the second (injection) position, causes the septum 1008 to be pierced by the proximal end of the hypodermic needle 1009. However, because the hypodermic needle 1009 does not pierce the septum 1008 in the pre-injection configuration, sterility of the medicament in the medicament container 1007 is maintained.

Safety shield 1019 includes a first protrusion 1410 on an inner surface thereof. Housing 1023 includes a corresponding second protrusion 1412 on an external surface thereof. As shown in FIG. 44A, in the pre-injection configuration, the first and second protrusions abut one another, thereby preventing the safety shield 1019 from moving any further in the proximal direction relative to the housing 1023 than the retracted position shown. Further, when the safety shield advances into the deployed position, the first protrusion 1410 will abut the sheer proximal-facing surface 1414a of the one-way catch 1414. Accordingly, advancement of the safety shield 1019 past the deployed position is prevented. In other words, the provision of the first protrusion 1410, second protrusion 1412, and one-way catch 1414, limit the travel of the safety shield 1019 to between the retracted position and the deployed position. Additionally, because of the sloped distal-facing surface 1414b of the one-way catch 1414, the first protrusion 1410 is able to pass over the one-way catch 1414 during assembly of the distal portion 1b of the injection device 1001. Because the one-way catch 1414 also includes a cantilever arm 1416, assembly of the distal portion 1b of the injection device 1001 is further facilitated. In short, the distal portion 1b of the injection device 1001 is easily assembled, but is not easily disassembled or tampered with.

As shown in FIG. 44C, housing 1023 further comprises assembly tabs 1418 on an external surface of a proximal portion thereof. The assembly tabs are configured to engage corresponding features of the handle 1003 when the injection device 1001 is assembled, thereby securing the distal portion 1b to the proximal portion 1a.

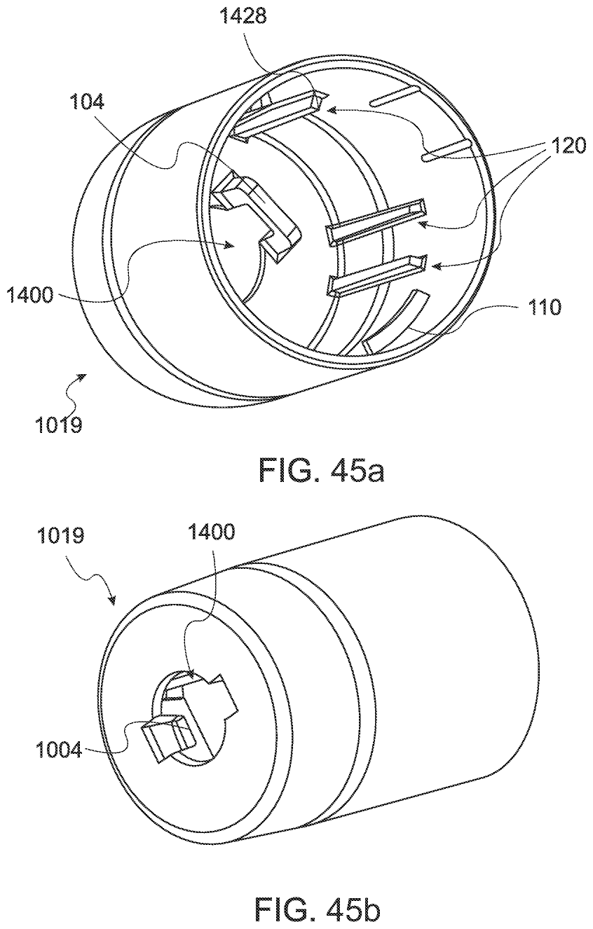

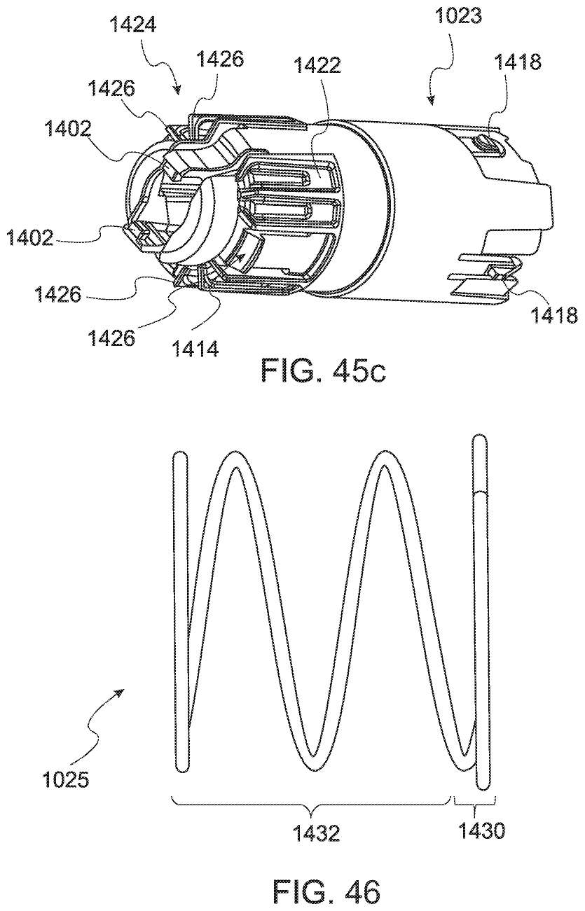

FIG. 45A shows a first perspective view of the safety shield 1019 from FIG. 1, in which the inner surfaces of the safety shield 1019 are visible. FIG. 45B shows a second perspective view of the safety shield 1019 from FIG. 1, in which the distal end is visible. FIG. 45C shows a perspective view of the housing 1023 from FIG. 1.

FIG. 45A shows one of two first protrusions 1410. Although not visible in FIG. 45A, a further first protrusion 1410 opposes the visible first protrusion 1410. In other words, the safety shield 1019 comprises two first protrusions 1410, respectively located on opposing inner surfaces of the safety shield 1019. As shown, the first protrusions 1410 are circumferential protrusions that extend around a portion of the inner circumference of the safety shield 1019. Also visible is one of two latching surfaces 1404 at the distal end of the safety shield. While not visible in FIG. 45A, a further latching surface 1404 opposes the visible latching surface 1404. In other words, the safety shield includes two latching surfaces 1404, respectively located adjacent opposing edges of the opening 1400. Finally, FIG. 45A shows longitudinal ridges 1420 which are formed on the inner surface of the safety shield 1019. When the safety shield 1019 is in its retracted position as shown in FIGS. 44a-44c, each longitudinal ridge 1420 is received within a respective longitudinal groove 1422 of the housing 1023.

As shown in FIG. 45C, housing 1023 further comprises a distal-facing shoulder 1424 which includes a plurality of distal-facing first locking surfaces 1426. The distal-facing first locking surfaces 1426 are located at the distal ends of the grooves 1422. Furthermore, each longitudinal ridge 1420 comprises a proximal-facing second locking surface 1428. That is to say, each longitudinal ridge 1420 extends distally from its respective proximal-facing second locking surface 1426. When the safety shield is in its deployed position, the ridges 1420 extend distally of the grooves 1422, and the first and second locking surfaces 1426, 1428 face one another.

FIG. 45C also shows the two latch arms 1402 of the housing 1023, and one of two one-way catches 1414. As the reader will understand, a further one-way catch is provided on the opposite side of the housing 1023 from the one-way catch that is visible. The two latch arms 1402 are configured to engage the two latching surfaces 1404. The two one-way catches 1414 are configured to engage the two circumferential protrusions 1410. Finally, two of four assembly tabs 1418 are visible.

FIG. 46 shows a side-view of the advancement spring 1025, in its natural (uncompressed) state. As shown, the advancement spring 1025 includes a proximal portion 1430 and a distal portion 1432. Proximal portion 1430 has a larger diameter than the distal portion 1432. When the injection device 1001 is assembled, the advancement spring 1025 is arranged between the housing 1023 and the safety shield 1019. Moreover, the advancement spring is arranged such that the proximal portion 1430 abuts the shoulder 1424 of the housing 1023, and such that the distal portion 1432 abuts the distal end of the safety shield 1019. Thus, when the safety shield 1019 is in the retracted position as shown in FIGS. 44a-44c, the advancement spring 1025 is in an axially compressed state, and biases the safety shield 1019 into the deployed position. Furthermore, when the safety shield 1019 is in the retracted position, the proximal portion 1430 of the advancement spring 1025 is located between the ridges 1420, and is thereby radially compressed by the ridges 1420. As is described in further detail below, when the safety shield 1019 is in the deployed position, the proximal portion 1430 of the advancement spring 1025 is no longer located between the ridges 1420, and therefore is no longer radially compressed. Accordingly, the proximal portion 1430 of the spring 1025 radially expands so as to become wedged between the first and second locking surfaces 1426, 1428. Accordingly, the safety shield 1019 is prevented from returning to the retracted position once it has reached its deployed position. Moreover, the one-way catch prevents the safety shield from moving further in the distal direction than the deployed position, and the proximal portion 1430 of the advancement spring 1025 wedges between the first and second locking surfaces 1426, 1428 to prevent the safety shield from moving in the proximal direction. The safety shield is jammed in the deployed position.

FIGS. 47A-47F show respectively the storage configuration of FIG. 1; the pre-injection configuration of FIGS. 2, 44A, 44B, 44C; a first mid-injection configuration; a first post-injection configuration; a second mid-injection configuration; and a second post-injection configuration. As described below, the second mid-injection configuration is a precursor to the second post-injection configuration.

As has been described above, in the storage configuration of FIG. 1, cover 1006 surrounds the housing 1023 and the safety shield 1019. This is storage configuration shown in FIG. 47A. When a user is ready to perform an injection, they will remove the cover 1006, thereby exposing the housing 1023 and safety shield 1019. This pre-injection configuration is shown in FIG. 47B. When a user performs an injection by actuating the drive assembly 1016, the medicament container 1007 advances distally relative to the needle hub 1011 until the hypodermic needle 1009 pierces the septum 1008, and the medicament container and needle hub 1011 will then move further distally so that the hypodermic needle 1009 pierces the injection site. This mid-injection configuration, in which the medicament container 1007 and the needle hub 1011 are in their second (injection) position, is shown in FIG. 47C.

As can be seen from FIGS. 47A-47C, the advancement spring 1025 is axially compressed. Accordingly, it is biasing the safety shield 1019 into the deployed position. However, in FIGS. 47A and 6B, the flexible latch arms 1402 engage the latching surfaces 1404, such that advancement of the safety shield into the deployed position. In other words, the safety shield 1019 is locked in the retracted position in the storage and pre-injection configurations. However, once the needle hub 1011 has moved into the second (injection) position as shown in the first mid-injection configuration of FIG. 47C, it disengages the flexible latch arms 1402 from the latching surfaces 1404. Accordingly, the safety shield 1019 is no longer locked in the retracted position.

If an injection is properly performed, the drive mechanism 1016 will be fully actuated, causing all medicament to be expelled from the medicament container 1007. Once this happens, the drive mechanism decouples from the plunger rod 1015. Accordingly, the return spring 1021 (which is compressed in the first mid-injection shown in FIG. 47C) causes the medicament container 1007 to return to its first (retracted) position, thus also retracting the hypodermic needle 1009. If the injection was performed properly by the user, the injection device 1001 would not have been removed from the injection site until the injection was complete and the medicament container 1007 had returned to its first (retracted) position. Therefore, if the injection was performed properly by the user, the latching arms 1402 would have reengaged the latching surfaces 1404 by the time the injection device 1001 was removed from the injection site. Accordingly, the safety shield 1019 would have been re-locked in the retracted position. In other words, the injection would end up in the first (correct) post-injection configuration shown in FIG. 47D, in which the needle 1009 is retracted back into the housing 1023 (thus rendering it safe) and the safety shield has not deployed. As also shown in FIG. 47D, when the medicament container 1007 and needle hub 1011 move proximally back to the first (retracted) position, they move a first indicator band 1434 in the proximal direction with them. The indicator band is located between the medicament container 1007 and the housing 1023 and may be green in colour. Accordingly, when the medicament container 1007 and needle hub 1011 move back to the first (retracted) position, the first indicator band 1434 becomes visible through a window portion 1436 of the housing 1023. The first indicator band 1434 thus becomes visible to the user, indicating delivery of a full dose of medicament through the needle 1009 and correct return of the medicament container 1007 and needle hub 1011 to the first (retracted) position.

If, on the other hand, the injection device were to be removed from the injection site prematurely, or if the needle retraction mechanism were to fail, then the injection device 1001 would end up in the second post-injection configuration, as illustrated in FIG. 47F, after transiently being in second mid-injection configuration illustrated in FIG. 47E.

In the second (incorrect) mid-injection configuration, either the drive assembly 1016 has not been fully actuated, thereby resulting in an incomplete dose of medicament being expelled from the medicament container 1007, or the drive assembly 1016 has not decoupled from the plunger rod 1015. In either case, the needle hub 1011 has not returned to the first (retracted) position, and so the latching arms 1402 remain in their unlocked position. In other words, the safety shield never 1019 returned to its locked configuration. Accordingly, removal of the injection device 1001 from the injection site allowed the safety shield 1019 to advance to its deployed position. The injection device 1001 transiently occupies the second mid-injection configuration of FIG. 47E, in which the needle remains in the second (injection) position but is nonetheless shielded from harm by the deployed safety shield 1019. Moreover, because the proximal portion 1430 of the advancement spring 1025 is wedged between the first and second locking surfaces 1426, 1428, return of the safety shield to the retracted position is prevented. In short, even though the device has been used incorrectly, the safety shield 1019 nonetheless renders the injection device safe. As can also be seen from FIG. 47E, a second indicator band 1438 that is located on an outer surface of a distal portion of the housing 1023, becomes visible due to advancement of the safety shield 1019. Therefore, because advancement of the safety shield 1019 results from incorrect use, visibility of the second indicator band 1438 also provides a convenient indication of this incorrect use. The second indicator band 1438 may be red in colour.

FIG. 47F shows the second (incorrect) post-injection configuration, in which the drive assembly 1016 has been fully activated, thereby expelling all medicament from the medicament container 1007; and in which the drive assembly 1016 has successfully decoupled from the plunger rod 1015, thereby enabling the return spring 1021 to return the medicament container 1007 to the first (retracted position), thereby retracting the needle 1009. However, because the injection device 1001 was removed from the injection site prior to retraction of the medicament container 1007 (as discussed in respect of the transient second mid-injection configuration of FIG. 47E), the safety shield 1019 is locked in its deployed position. Moreover, even though all of the medicament has been delivered through the hypodermic needle 1009, the injection device 1001 was prematurely removed from the injection site, and so the complete dose of medicament has not been delivered to the injection site. As also shown in FIG. 47F, both the first indicator band 1434, and the second indicator band 1438, are showing. This indicates that a complete dose has been delivered through the needle (due to the visibility of the first indicator band 1434); but also that the injection device 1001 was prematurely removed (due to the visibility of the second indicator band 1438) such that the compete dose was not delivered to the patient.

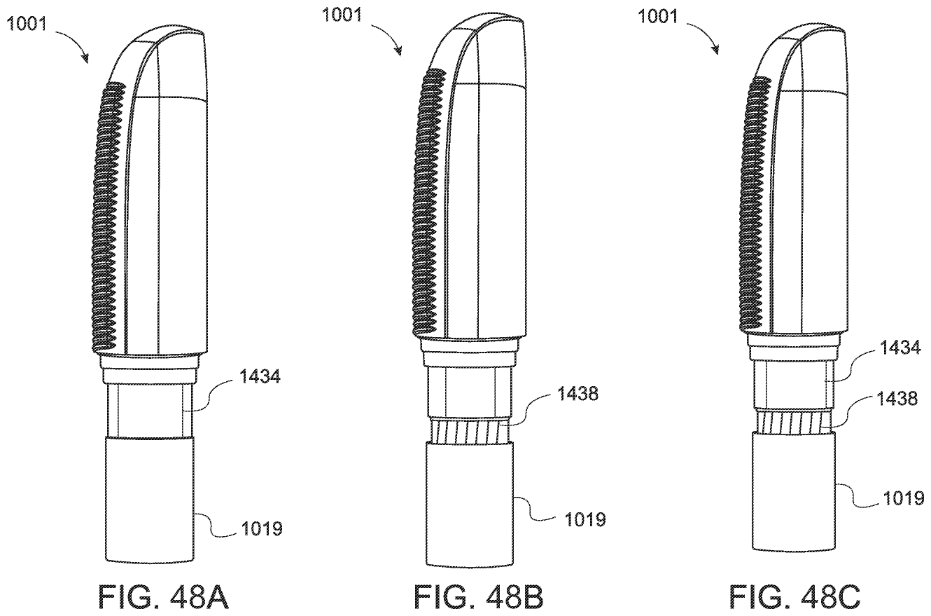

In short, FIGS. 47D-47F and 48A-48C illustrate the following configurations:
A first post-injection configuration, indicating correct use of the injection device 1001. In this configuration, the needle 1009 is in the first (retracted) position, and the safety sleeve 1019 is in the retracted position. This configuration is shown in FIG. 47D. See also FIG. 48A, which shows an external view of the injection device 1001 in the first post-injection configuration. As shown, the first indicator band 1434 is visible.
A second mid-injection configuration, indicating incorrect use of injection device 1001 and incomplete delivery of the medicament through the needle 1009. In this configuration, the needle 1009 is in the injection position, and the safety sleeve 1019 is in the deployed position. This configuration is shown in FIG. 47E. See also FIG. 48B, which shows an external view of the injection device 1001 in the second mid-injection configuration. As shown, the second indicator band 1438 is visible.
A second post-injection configuration, indicating incorrect use of injection device 1001, complete delivery of the medicament through the needle 1009, and incomplete delivery of the medicament to the injection site. In this configuration, the needle 1009 is in the first (retracted) position, and the safety sleeve 1019 is in the deployed position. This configuration is shown in FIG. 47F. See also FIG. 48C, which shows an external view of the injection device 1001 in the second post-injection configuration. As shown, the first indicator band 1434 and the second indicator band 1438 are visible.

In the unlikely event that the medicament container retraction mechanism failed, then the second mid-injection configuration may be a third post-injection configuration. This is very unlikely. But even so, the needle is nonetheless rendered safe by the deployment of the safety shield.

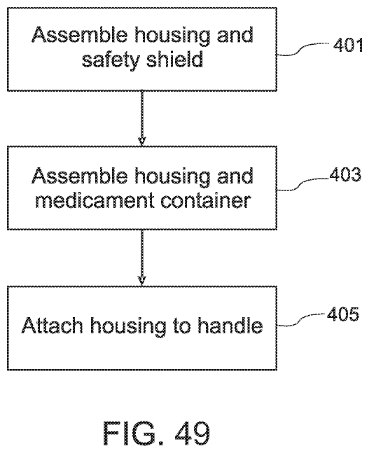

FIG. 49 is shows a method of assembling the injection device of FIG. 1.

At step 401, the advancement spring 1025 is placed into the safety shield 1019 so that it is retained within the longitudinal ridges 1420; and the distal end of the housing 1023 is then inserted into the safety shield 1019, so that the advancement spring 1025 is located between the housing 1023 and the safety shield 1019. The housing 1023 is advanced into the safety shield 1019 until the protrusion 1410 passes over the one-way catch 1414, such that separation of the safety shield 1019 from the housing 1023 is prevented. The housing 1023 is further advanced into the safety shield 1019 until the flexible latch arms 1402 engage the latching surfaces 1404 to thereby lock the safety shield 1019 in the retracted position. A tool may be inserted into the proximal end of the housing 1023 during this step, to splay the flexible latch arms 1402 during assembly of the housing 1023 and safety shield 1019. By removing the tool once the housing 1023 is further advanced into the safety shield 1019, the flexible latch arms 1402 will correctly engage the latching surfaces 1404 to thereby lock the safety shield 1019 in the retracted position.

At step 403, the return spring 1021 is inserted into the housing 1023, followed by the medicament container 1007. Accordingly, the return spring 1021 is located between the housing 1023 and the medicament container 1007. When the medicament container 1007 is inserted into the housing 1023, it is already attached to the needle hub 1011 and hypodermic needle 1009. Moreover, the needle 1009 is already encased by the needle shield 1004 and needle cap 1005.

As the reader will appreciate, steps 401 and 403 could be performed in reverse order. That is to say, step 704 could be performed before step 401. When step 403 is performed first, the tool may not be used to splay the flexible latch arms 1402.

At step 405, the distal portion 1b as assembled in steps 401-403 is attached to the handle 1003, thereby assembling the full injection device 1001.

Also disclosed herein are a number of examples according to the following numbered clauses.
A1. An injection device, or sub-assembly for an injection device, comprising:
   a housing defining a longitudinal axis;
   a drive spring disposed within the housing, the drive spring having a distal end and a proximal end opposite the distal end along the longitudinal axis, and the drive spring defining an interior cavity;
   a plunger disposed at least partially within a medicament container
   a plunger rod engaged with the plunger; and
   a drive-lock mechanism comprising:
   a latch mechanism at least partially received within the interior cavity and comprising at least one engagement portion configured to releasably engage the plunger rod, wherein the latch mechanism is configured to move distally under action of the drive spring; and
   a retraction collar engaged with the latch mechanism, o wherein the latch mechanism is configured to move from a drive-locked position to a drive-unlocked position relative to the retraction collar;
   wherein in the drive-locked position, the retraction collar holds the at least one engagement portion in engagement with the plunger rod such that extension of the drive spring moves the latch mechanism and retraction collar distally to expel medicament from a syringe, and
   in the drive-unlocked position, the retraction collar does not hold the at least one engagement portion in engagement with the plunger rod, thus permitting the at least one engagement portion to disengage the plunger.
A2. The injection device of clause A1, wherein the latch mechanism comprises a distal flange in contact with the distal end of the drive spring.
A3. The injection device of any preceding clause, wherein the latch mechanism and the retraction collar are configured to move distally in tandem when the latch mechanism is in the drive-locked position.
A4. The injection device of clause A3, wherein the housing defines an abutment configured to prevent distal movement of the retraction collar beyond the abutment and thereby allow the latch mechanism to move distally relative to the retraction collar to transition the drive-lock mechanism from the drive-locked position to the drive-unlocked position.
A5. The injection device of any preceding clause, wherein in the drive-locked position, the latch mechanism is configured to move distally with the retraction collar due to frictional engagement between the latch mechanism and the retraction collar.
A6. The injection device of clause A5, wherein the latch mechanism is configured to move from the drive-locked position to the drive-unlocked position by overcoming the friction between the latch mechanism and the retraction collar due to force applied by the drive spring.
A7. The injection device of any preceding clause, wherein the latch mechanism is configured to retain the drive spring in a compressed condition prior to activation of the injection device.
A8. The injection device of clause A7, wherein the latch mechanism defines at least one engagement element configured to be releasably secured to a portion of the housing to retain the drive springe in the compressed condition.
A9. The injection device of any preceding clause, wherein the latch mechanism comprises a latch extension configured to engage with the drive spring and a latch engaged with the latch extension.
A10. The injection device of clause A9, wherein the latch comprises the at least one engagement portion and the latch extension comprises the at least one engagement element.
A11. The injection device of clause A10, wherein the latch mechanism has a monolithic body.
A12. The injection device of clause A7, further comprising a handle, wherein the handle is axially movable relative to the housing from an inactive position to an active position, wherein movement of the housing from the inactive position to the active position releases the drive spring from a compressed condition.
A13. The injection device of clause A12, wherein the movement of the housing from the inactive position to the active position upon compression of the distal end of the device against injectable tissue is configured to release the drive spring from the compressed condition.
A14. The injection device of clause A13, further comprising an actuator spring disposed between the housing and the handle, wherein the actuator spring biases the housing towards the inactive position.
A15. The injection device of clause A13, wherein the at least one engagement element is secured between the housing and an actuator in the inactive position.
A16. The injection device of clause A15, wherein the actuator is disposed entirely within the handle.
A17. The injection device of clause A16, wherein the actuator is axially fixed to the handle.
A18. The injection device of any preceding clause, wherein the retraction collar comprises a sleeve including at least one opening.
A19. The injection device of clause A18, wherein when the latch mechanism is in the drive-locked position, the sleeve of the retraction collar is radially aligned with the at least one engagement portion to hold the at least one engagement portion in engagement with the plunger rod, and in the drive-unlocked position, the at least one opening is radially aligned with the at least one engagement portion, thus allowing the at least one engagement portion to flex outwards and disengage from the plunger rod.
A20. The injection device of any preceding clause, wherein the drive spring defines a single spring configured to 1) move the syringe axially through the housing, and 2) expel the medicament from the syringe.
A21. A method of manufacturing an injection device, or sub-assembly for an injection device, the method comprising:
   providing a housing defining a longitudinal axis;
   disposing a drive spring within the housing, the drive spring having a distal end and proximal end opposite the distal end along the longitudinal axis, the drive spring defining an interior cavity;
   disposing a plunger at least partially within a medicament container;
   engaging a plunger rod with the plunger;
   engaging a latch mechanism with a retraction collar to form a drive-lock mechanism, the latch mechanism comprising at least one engagement portion;
   disposing the latch mechanism at least partially within the interior cavity and releasably engaging the at least one engagement portion with the plunger rod; and
   arranging the retraction collar in a drive-locked position such that the retraction collar holds the at least one engagement portion in engagement with the plunger rod and extension of the drive spring moves the latch mechanism and retraction collar distally to expel medicament from a syringe, wherein the retraction collar is configured to move from the drive-locked position to a drive-unlocked position wherein the retraction collar does not hold the at least one engagement portion in engagement with the plunger rod.
A22. The method of claim A21, further comprising:
compressing the distal and proximal ends of the drive spring into a compressed condition, and configuring the latch mechanism to retain the drive spring in the compressed condition.
B1. An injection device, or sub-assembly for an injection device, comprising:
   a housing defining a longitudinal axis;
   a drive spring disposed within the housing;
   a first drive component configured to transfer drive from the drive spring to a plunger disposed within a medicament container;
   a damper concentrically arranged with respect to the first drive component, wherein the damper is longitudinally fixed relative to the housing;
   wherein the first drive component is configured to move along the longitudinal axis relative to the damper under the influence of the drive spring; and
   wherein the damper is configured to frictionally engage a surface of the first drive component during movement of the drive component relative to the damper.
B2. The injection device of clause B1, wherein the damper is annular.
B3. The injection device of clause B1 or clause B2, wherein the drive spring is configured to advance a medicament container from a retracted position to an extended position relative to the housing.
B4. The injection device of clause B3, wherein the damper is attached to the housing via a longitudinally-extending pin.
B5. The injection device of clause B4, wherein a distal end of the damper defines a socket configured to receive a corresponding head of a tool for affixing the damper to the pin.
B6. The injection device of any of clauses B1-B5, wherein the damper comprises:
an elongate body fixed within the housing; and
wherein the first drive component comprises an inner wall defining a channel, and the damper is configured to be received by the channel and to engage the inner wall of the first drive component.
B7. The injection device of any preceding clause, wherein the damper further comprises at least one deformable damping member disposed on the elongate body.
B8. The injection device of clause B7, wherein the elongate body comprises a head part comprising at least one circumferential groove located at a distal end, the at least one circumferential groove configured to engage a complementary portion of the damping member.
B9. The injection device of clause B6 or B7, wherein the channel has at least two different diameters along the longitudinal axis, such that a magnitude of frictional engagement between the first drive component and the damper changes as the first drive component moves relative to the damper.
B10. The injection device of any of clauses B7-B9, wherein the deformable damping member comprises an overmolded component.
B11. The injection device of any of clauses B6-B10, wherein the inner wall of the first drive component comprises a plurality of grooves extending in a longitudinal direction, optionally along the longitudinal axis or parallel thereto.
B12. The injection device of clause B11, wherein the plurality of grooves comprises at least one groove having a first length and at least one groove having a second length, wherein the first and second lengths are different.
B13. The injection device of clause B11 or clause B12, wherein the grooves are circumferentially spaced apart about the longitudinal axis.
B14. The injection device of any of clauses B10-B13, wherein a percentage of the inner wall comprised by the grooves decreases in a distal direction.
B15. A method of manufacturing an injection device, or sub-assembly for an injection device, the method comprising:
   providing a housing defining a longitudinal axis;
   attaching a damper to the housing such that the damper is translationally fixed relative to the housing along the longitudinal axis;
   disposing a drive spring within the housing; and
   disposing a first drive component within the housing such that the damper is arranged concentrically within the first drive component, wherein the first drive component is configured to be driven by the drive spring along the longitudinal axis relative to the damper such that the damper and the first drive component frictionally engage.
B16. The method of clause B15, wherein the damper comprises an elongate body, and a damping member, and wherein the method further comprises;
   overmolding a deformable damping member onto the elongate body to form the damper.
C1. An assembly for an injection device for injecting a medicament, the assembly comprising:
   a container containing the medicament and having a cap, the container sealed by a septum;
   a sealing element in contact with an external surface of the cap of the container;
   a needle for piercing the septum; and
   a needle hub attached to the needle, wherein the sealing element is disposed between the external surface of the cap of the container and an internal surface of the needle hub;
   wherein the assembly is configured to transition from a first state, in which the needle is held away from the septum and a free end of the needle sits in a cavity sealed by the sealing element, to a second state, in which the needle passes through the septum, and wherein a seal between the internal surface of the needle hub and the external surface of the cap is provided by the sealing element when the assembly is in the first state and wherein the seal is maintained throughout transition from the first state to the second state and is maintained when the assembly is in the second state.
C2. The assembly as claimed in clause C1, the assembly comprising:
   a housing defining a longitudinal axis and configured to receive the container containing the medicament, the housing having a distal end which defines an opening for receiving a portion of the needle;
   a safety shield surrounding at least a distal portion of the housing, the safety shield configured to advance distally relative to the housing from a retracted position, to a deployed position for shielding the needle; and
   an advancement spring arranged between the housing and the safety shield, the advancement spring configured to advance the safety shield from the retracted position to the deployed position.
C3. The assembly as claimed in clause C2, further comprising a releasable locking mechanism configured, when engaged, to lock the safety shield in the retracted position.
C4. The assembly as claimed in clause C3, wherein the needle hub is moveable relative to the housing between a first position in which it is recessed from the opening, and a second position in which it extends through the opening; and wherein the needle hub is configured to disengage the releasable locking mechanism when in the second position, thereby unlocking the safety shield.
C5. The assembly as claimed in clause C3 or C4, wherein the releasable locking mechanism comprises:
   a latching surface connected to the safety shield, and a flexible latch arm connected to the housing or a latching surface connected to the housing, and a flexible latch arm connected to the safety shield;
   wherein the flexible latch arm is configured to engage the latching surface to thereby lock the safety shield in the retracted position; and
   wherein the needle hub is configured to disengage the flexible latch arm from the latching surface when in the second position.
C6. The assembly as claimed in any of clauses C2 to C5, wherein the advancement spring is configured to interlock with the housing and the safety shield when the safety shield is in the deployed position, to thereby prevent return of the safety shield to the retracted position.
C7. The assembly as claimed in any preceding clause, wherein the sealing element is chemically bonded to the external surface of the cap.
C8. The assembly as claimed in any preceding clause, wherein the sealing element is over-moulded onto the external surface of the cap.
C9. The assembly as claimed in any of clauses C1 to C6, wherein the sealing element is an O-ring.
C10. The assembly as claimed in any of clauses C1 to C7, wherein the sealing element comprises a first material, the cap comprises a second material different to the first material, and the sealing element and cap define a monolithic body formed via two-shot injection moulding.
C11. The assembly as claimed in any preceding clause, wherein the needle hub defines a first inner surface extending perpendicular to the needle and facing the cap, a proximal protrusion which extends inwards, towards the needle, and a second inner surface extending parallel to the needle from the first inner surface to the proximal protrusion, wherein the second inner surface is configured to engage the sealing element in the first and second states and during transition therebetween.
C12. The assembly as claimed in any preceding clause, wherein the cap comprises a first rib and a second rib, wherein the sealing element is disposed between the first and second ribs.
C13. The assembly as claimed in clause C12,
wherein, when the assembly is in the first state, a distance between the first rib and the free end of the needle is less than a distance between the second rib and the free end of the needle; wherein when the assembly is in the first state, the proximal protrusion of the needle hub engages the second rib of the cap, and when the assembly is in the second state, the first inner surface of the needle hub engages the first rib of the cap.
C14. The assembly as claimed in any preceding clause, wherein the cap has a first positioning recess within which the sealing element is disposed and a second positioning recess configured to selectively receive a corresponding positioning protrusion of the needle hub when the assembly is in the second state.
C15. The assembly as claimed in clause C14, wherein a surface of the positioning protrusion of the needle hub is in contact with the sealing element when the assembly is in the first state.
C16. A method of manufacturing an assembly for an injection device for injecting a medicament, the method comprising providing:
   a container having a cap, the container sealed by a septum;
   a sealing element in contact with an external surface of the cap of the container;
   a needle for piercing the septum; and
   a needle hub, wherein the sealing element is disposed between the external surface of the cap of the container and an internal surface of the needle hub;
   wherein the assembly is configured to transition from a first state, in which the needle is held away from the septum and a free end of the needle sits in a cavity sealed by the sealing element, to a second state, in which the needle passes through the septum, and wherein a seal between the internal surface of the needle hub and the external surface of the cap is provided by the sealing element when the assembly is in the first state and wherein the seal is maintained throughout transition from the first state to the second state and is maintained when the assembly is in the second state.
C17. The method of manufacturing an assembly as claimed in clause C16, the method comprising: providing a housing defining a longitudinal axis;
   providing a safety shield surrounding at least a distal portion of the housing, the safety shield being distally advanceable relative to the housing from a retracted position, to a deployed position for shielding the needle; and
   arranging an advancement spring between the housing and the safety shield, the advancement spring configured to advance the safety shield.
C18. The method of manufacturing an assembly as claimed in clause C16 or C17 comprising providing a releasable locking mechanism configured, when engaged, to lock the safety shield in the retracted position.
C19. The method of manufacturing an assembly as claimed in clause C18, wherein the needle hub is moveable relative to the housing between a first position in which it is recessed from the opening, and a second position in which it extends through the opening; and wherein the needle hub is configured to disengage the releasable locking mechanism when in the second position, thereby unlocking the safety shield.
C20. The method of manufacturing an assembly as claimed in clause C18 or C19, wherein the releasable locking mechanism comprises:
   a latching surface connected to the safety shield, and a flexible latch arm connected to the housing or a latching surface connected to the housing, and a flexible latch arm connected to the safety shield;
   wherein the flexible latch arm is configured to engage the latching surface to thereby lock the safety shield in the retracted position; and
   wherein the needle hub is configured to disengage the flexible latch arm from the latching surface when in the second position.
C21. The method of manufacturing an assembly as claimed in any of clauses C17 to C20 wherein the advancement spring is configured to interlock with the housing and the safety shield when the safety shield is in the deployed position, to thereby prevent return of the safety shield to the retracted position
C22. The method of manufacturing an assembly as claimed in any of clauses C16 to C21, wherein providing the sealing element comprises chemically bonding the sealing element to the external surface of the cap.
C23. The method of manufacturing an assembly as claimed in any of clauses C16 to C22 wherein providing the sealing element comprises over-moulding the sealing element onto the external surface of the cap.
C24. The method of manufacturing an assembly as claimed in any of clauses C16 to C23, wherein the sealing element comprises a first material and the cap comprises a second material that is different than the first material.
C25. The method of manufacturing an assembly as claimed in clause C22, wherein chemically bonding comprises performing a 2-shot injection moulding process.
C26. A method of manufacturing an assembly as claimed in any of clauses C16 to C21 or C24, wherein the sealing element is an O-ring.
D1. An injection device, or sub-assembly for an injection device, comprising:
   a housing defining a longitudinal axis and configured to receive a medicament container containing a medicament, the housing having a distal end which defines an opening for receiving a portion of a needle operably coupled with the medicament container;
   a safety shield surrounding at least a distal portion of the housing, the safety shield configured to advance distally relative to the housing from a retracted position, to a deployed position for shielding the needle; and
   an advancement spring arranged between the housing and the safety shield, the advancement spring configured to advance the safety shield from the retracted position to the deployed position; wherein the advancement spring is configured to interlock with the housing and the safety shield when the safety shield is in the deployed position, to thereby prevent return of the safety shield to the retracted position.
D2. The injection device of clause D1, wherein:
the housing comprises a first locking surface, and the safety shield comprises a second locking surface arranged to face the first locking surface when the safety shield is in the deployed position; and
the advancement spring is configured to interlock with the first and second locking surfaces when the safety shield is in the deployed position, to thereby prevent return of the safety shield to the retracted position.
D3. The injection device of clause D2, wherein:
the first locking surface comprises a distal-facing shoulder of the housing, and the second locking surface comprises a proximal-facing surface from which a longitudinal ridge extends in the distal direction;
the longitudinal ridge is configured to radially compress the advancement spring when the safety shield is in the retracted position; and
the advancement spring is configured to radially expand as it passes the second locking surface during advancement of the safety shield.
D4. The injection device of any preceding clause, wherein the advancement spring comprises a proximal portion having a first diameter, and a distal portion having a second diameter that is smaller than the first diameter, wherein the proximal portion is configured to interlock with the housing and the safety shield when the safety shield is in the deployed position.
D5. The injection device of clause D4, wherein the proximal portion of the advancement spring is compressed into a third diameter that is smaller than the first diameter when the safety shield is in the retracted position, and wherein the advancement spring is arranged to expand to the first diameter when safety shield is in the deployed position.
D6. An injection device of any preceding clause, wherein:
   one of the housing and the safety shield comprises a retaining tab, and the other of the housing and the safety shield comprises a one-way catch; and
wherein the retaining tab is configured to slide over the one-way catch during assembly of the injection device, and is configured to abut the one-way catch during advancement of the safety shield to thereby prevent separation of the safety shield from the housing.
D7. The injection device of clause D6, wherein the one-way catch comprises a sloped surface arranged to facilitate the movement of the retaining tab over the one-way catch during assembly, and comprises a sheer surface arranged to prevent the movement of the retaining tab over the one-way catch during advancement of the safety shield.
D8. The injection device of clause D7, wherein the one-way catch further comprises a cantilever arm.
D9. The injection device according to any of clauses D6 to D8, wherein the housing comprises the one-way catch, and the safety shield comprises the retaining tab.
D10. The injection device of clause D9, wherein the retaining tab comprises a circumferential protrusion.
D11. The injection device of any preceding clause, further comprising a releasable locking mechanism configured, when engaged, to lock the safety shield in the retracted position.
D12. The injection device of clause D11, further comprising a needle hub coupled to the needle and the medicament container;
wherein the needle hub is moveable relative to the housing between a first position in which it is recessed from the opening, and a second position in which it extends through the opening; and
wherein the needle hub is configured to disengage the releasable locking mechanism when in the second position, thereby unlocking the safety shield.
D13. The injection device of clause D12, wherein the releasable locking mechanism comprises:
   a latching surface connected to one of the safety shield and the housing, and a flexible latch arm connected to the other of the safety shield and the housing;
   wherein the flexible latch arm is configured to engage the latching surface to thereby lock the safety shield in the retracted position; and
   wherein the needle hub is configured to disengage the flexible latch arm from the latching surface when in the second position.
D14. The injection device according to any of clauses D11 to D13, further comprising a return spring arranged to bias the medicament container and needle hub into the first position.
D15. The injection device of clause D14, wherein the return spring comprises a helical spring and is positioned between the housing and the medicament container.
D16. The injection device of any preceding clause, wherein the advancement spring comprises a helical spring.
D17. A method of manufacturing an injection device, the method comprising:
   providing a housing defining a longitudinal axis;
   providing a safety shield surrounding at least a distal portion of the housing, the safety shield being distally advanceable relative to the housing from a retracted position, to a deployed position for shielding the needle; and
   arranging an advancement spring between the housing and the safety shield, the advancement spring configured to advance the safety shield;
wherein the advancement spring is configured to interlock with the housing and the safety shield when the safety shield is in the deployed position, to thereby prevent return of the safety shield to the retracted position.
D18. The method of clause D17, wherein one of the housing and the safety shield comprises a retaining tab, and the other of the housing and the safety shield comprises a one-way catch;
wherein the method comprises sliding the safety shield over the housing and towards the retracted position, such that the retaining tab passes over the one-way catch until it locks in place behind the one-way catch to thereby prevent separation of the safety shield from the housing.
D19. The method of clause D18, further comprising positioning the advancement spring within the safety shield before sliding the safety shield onto the housing.
E1. An injection device comprising:
   a housing defining a longitudinal axis, and configured to receive a medicament container containing a medicament, the housing having a distal end which defines an opening for receiving a portion of a needle operably coupled with the medicament container;
   a drive spring disposed within the housing, the drive spring having a distal end and a proximal end opposite the distal end along the longitudinal axis, and the drive spring defining an interior cavity;
   a plunger disposed at least partially within a medicament container;
   a plunger rod engaged with the plunger; and
   wherein the injection device comprises one or more of:
      a) a drive-lock mechanism comprising:
         a latch mechanism at least partially received within the interior cavity and comprising at least one engagement portion configured to releasably engage the plunger rod, wherein the latch mechanism is configured to move distally under action of the drive spring; and
         a retraction collar engaged with the latch mechanism,
         wherein the latch mechanism is configured to move from a drive-locked position to a drive-unlocked position relative to the retraction collar;
         wherein in the drive-locked position, the retraction collar holds the at least one engagement portion in engagement with the plunger rod such that extension of the drive spring moves the latch mechanism and retraction collar distally to expel medicament from a syringe, and
         in the drive-unlocked position, the retraction collar does not hold the at least one engagement portion in engagement with the plunger rod, thus permitting the at least one engagement portion to disengage the plunger;
      b) a damping mechanism comprising:
         a first drive component, optionally the plunger rod, configured to transfer drive from the drive spring to the plunger disposed within a medicament container;
         a damper concentrically arranged with respect to the first drive component, wherein the damper is longitudinally fixed relative to the housing;
         wherein the first drive component is configured to move along the longitudinal axis relative to the damper under the influence of the drive spring; and
         wherein the damper is configured to frictionally engage a surface of the first drive component during movement of the drive component relative to the damper;
      c) a safety shield arrangement comprising:
         a safety shield surrounding at least a distal portion of the housing, the safety shield configured to advance distally relative to the housing from a retracted position, to a deployed position for shielding the needle; and
         an advancement spring arranged between the housing and the safety shield, the advancement spring configured to advance the safety shield from the retracted position to the deployed position;
         wherein the advancement spring is configured to interlock with the housing and the safety shield when the safety shield is in the deployed position, to thereby prevent return of the safety shield to the retracted position; and
      d) container connection assembly comprising:
         a container containing the medicament and having a cap, the container sealed by a septum;
         a sealing element in contact with an external surface of the cap of the container;
         a needle for piercing the septum; and
         a needle hub attached to the needle, wherein the sealing element is disposed between the external surface of the cap of the container and an internal surface of the needle hub;
         wherein the assembly is configured to transition from a first state, in which the needle is held away from the septum and a free end of the needle sits in a cavity sealed by the sealing element, to a second state, in which the needle passes through the septum, and wherein a seal between the internal surface of the needle hub and the external surface of the cap is provided by the sealing element when the assembly is in the first state and wherein the seal is maintained throughout transition from the first state to the second state and is maintained when the assembly is in the second state.

It will be understood that, where used, the terms “proximal” and “distal” are used for convenience in interpreting the drawings and are not to be construed as limiting. The term “distal” refers to a direction towards the injection site (or the end of the needle for contacting the patient at the injection site) and the term “proximal” refers to a direction away from the injection site (or the end of the needle for contacting the patient at the injection site). The term “comprising” should be interpreted as meaning “including but not limited to”, such that it does not exclude the presence of features not listed. Where the term ‘annular’ is used, it should not be taken to be limited to a circular shape only, but rather to refer to an uninterrupted perimeter of any shape. The term ‘longitudinal’ should be taken to refer to the longitudinal axis along which the needle is disposed. Similarly, radial refers to a direction perpendicular to the longitudinal axis of the needle. Radially outwards refers to a direction away from the needle and radially inwards should be taken to mean towards the needle.

The embodiments described and shown in the accompanying drawings above are provided as examples of ways in which the invention may be put into effect and are not intended to be limiting on the scope of the invention. Modifications may be made, and elements may be replaced with functionally and structurally equivalent parts and features of different embodiments may be combined without departing from the disclosure.

1. An injection device comprising:
a housing defining a longitudinal axis and configured to receive a medicament container containing a medicament, the housing having a distal end which defines an opening for receiving a portion of a needle operably coupled with the medicament container;
a safety shield surrounding at least a distal portion of the housing, the safety shield configured to advance distally relative to the housing from a retracted position, to a deployed position for shielding the needle; and
an advancement spring arranged between the housing and the safety shield, the advancement spring configured to advance the safety shield from the retracted position to the deployed position,
wherein the advancement spring is configured to interlock with the housing and the safety shield when the safety shield is in the deployed position, to thereby prevent return of the safety shield to the retracted position.

2. The injection device of claim 1, wherein:
the housing comprises a first locking surface, and the safety shield comprises a second locking surface arranged to face the first locking surface when the safety shield is in the deployed position; and
the advancement spring is configured to interlock with the first and second locking surfaces when the safety shield is in the deployed position, to thereby prevent return of the safety shield to the retracted position.

3. The injection device of claim 2, wherein:
the first locking surface comprises a distal-facing shoulder of the housing, and the second locking surface comprises a proximal-facing surface from which a longitudinal ridge extends in the distal direction;
the longitudinal ridge is configured to radially compress the advancement spring when the safety shield is in the retracted position; and
the advancement spring is configured to radially expand as it passes the second locking surface during advancement of the safety shield.

4. The injection device of claim 1, wherein the advancement spring comprises a proximal portion having a first diameter, and a distal portion having a second diameter that is smaller than the first diameter, wherein the proximal portion is configured to interlock with the housing and the safety shield when the safety shield is in the deployed position.

5. The injection device of claim 4, wherein the proximal portion of the advancement spring is compressed into a third diameter that is smaller than the first diameter when the safety shield is in the retracted position, and wherein the advancement spring is arranged to expand to the first diameter when the safety shield is in the deployed position.

6. The injection device of claim 1, wherein:
one of the housing and the safety shield comprises a retaining tab, and the other of the housing and the safety shield comprises a one-way catch; and
wherein the retaining tab is configured to slide over the one-way catch during assembly of the injection device, and is configured to abut the one-way catch during advancement of the safety shield to thereby prevent separation of the safety shield from the housing.

7. The injection device of claim 6, wherein the one-way catch comprises a sloped surface arranged to facilitate movement of the retaining tab over the one-way catch during assembly, and comprises a sheer surface arranged to prevent movement of the retaining tab over the one-way catch during advancement of the safety shield.

8. The injection device of claim 7, wherein the one-way catch further comprises a cantilever arm.

9. The injection device of claim 6, wherein the housing comprises the one-way catch, and the safety shield comprises the retaining tab.

10. The injection device of claim 9, wherein the retaining tab comprises a circumferential protrusion.

11. The injection device of claim 1, further comprising a releasable locking mechanism configured, when engaged, to lock the safety shield in the retracted position.

12. The injection device of claim 11, further comprising a needle hub coupled to the needle and the medicament container;
wherein the needle hub is moveable relative to the housing between a first position in which it is recessed from the opening, and a second position in which it extends through the opening; and
wherein the needle hub is configured to disengage the releasable locking mechanism when in the second position, thereby unlocking the safety shield.

13. The injection device of claim 12, wherein the releasable locking mechanism comprises:
a latching surface connected to one of the safety shield and the housing, and a flexible latch arm connected to the other of the safety shield and the housing;
wherein the flexible latch arm is configured to engage the latching surface to thereby lock the safety shield in the retracted position; and
wherein the needle hub is configured to disengage the flexible latch arm from the latching surface when in the second position.

14. The injection device of claim 12, further comprising a return spring arranged to bias the medicament container and the needle hub into the first position.

15. The injection device of claim 14, wherein the return spring comprises a helical spring and is positioned between the housing and the medicament container.

16. The injection device of claim 1, wherein the advancement spring comprises a helical spring.

17. A method of manufacturing an injection device, the method comprising:
providing a housing defining a longitudinal axis;
providing a safety shield surrounding at least a distal portion of the housing, the safety shield being distally advanceable relative to the housing from a retracted position, to a deployed position; and
arranging an advancement spring between the housing and the safety shield, the advancement spring configured to advance the safety shield,
wherein the advancement spring is configured to interlock with the housing and the safety shield when the safety shield is in the deployed position, to thereby prevent return of the safety shield to the retracted position.

18. The method of claim 17, wherein one of the housing and the safety shield comprises a retaining tab, and the other of the housing and the safety shield comprises a one-way catch; and
wherein the method further comprises sliding the safety shield over the housing and towards the retracted position, such that the retaining tab passes over the one-way catch until it locks in place behind the one-way catch to thereby prevent separation of the safety shield from the housing.

19. The method of claim 18, further comprising positioning the advancement spring within the safety shield before sliding the safety shield onto the housing.
